(19) United States
(12) Patent Application Publication
(10) Pub. No.: US 2025/0134919 A1
(43) Pub. Date: May 1, 2025

(54) OPTIMIZED SEQUENCES FOR ENHANCED tRNA EXPRESSION OR/AND NONSENSE MUTATION SUPPRESSION

(71) Applicant: University of Rochester, Rochester, NY (US)

(72) Inventors: John D. Lueck, Rochester, NY (US); Joseph J. Porter, Rochester, NY (US)

(73) Assignee: University of Rochester, Rochester, NY (US)

(21) Appl. No.: 18/835,570

(22) PCT Filed: Feb. 6, 2023
(86) PCT No.: PCT/US2023/062053

§ 371 Date: Aug. 2, 2024

Related U.S. Application Data
(60) Provisional application No. 63/307,338, filed on Feb. 7, 2022.

(51) Int. Cl. A61K 31/7105; A61K 48/00; A61P 27/02; C12N 15/113; C12N 15/67

(52) U.S. Cl. CPC A61K 31/7105; A61K 48/005; A61P 27/02; C12N 15/113; C12N 15/67; C12N 2330/50; C12N 2510/00

(57) ABSTRACT

The present disclosure relates to nucleic acids, compositions, and methods for treating a disease or disorder associated with premature termination codon. Certain aspects of the disclosure relate to polynucleotides, vectors, and host cells, and uses thereof.

### CROSS REFERENCE TO RELATED APPLICATION

This application claims priority to U.S. Provisional Application No. 63/307,338 filed on Feb. 7, 2022. The content of the application is incorporated herein by reference in its entirety.

### GOVERNMENT INTERESTS

This invention was made with government support under 5R01HL153988 awarded by the National Institutes of Health. The government has certain rights in the invention.

### FIELD OF THE INVENTION

This invention relates to anti-codon edited-tRNAs based agents and methods for treating disorders associated with premature termination codon.

### BACKGROUND

The genetic code is a composed of nucleotide triplets called codons, which specify the amino acid added during protein synthesis. Of the 64 combinations of nucleotides making up a codon, 61 code for the insertion of an amino acid into the growing polypeptide chain on the ribosome, while three (TAA, TAG, and TGA) are reserved to signal for translation termination. A mutation (generally a single-nucleotide substitution) resulting in the conversion of a codon originally coding for an amino acid to one of the termination codons is termed a nonsense mutation, which results in a premature termination codon (PTC) in the protein coding sequence. The truncated protein product resulting from genes containing PTCs often leads to a loss in the protein function. Nonsense mutations account for 10-15% of all genetic lesions leading to disease, resulting in nearly 1000 serious genetic disorders⁶. For example, approximately 12 percent of the cystic fibrosis (CF) community are affected by nonsense mutations in the Cystic Fibrosis Transmembrane conductance Regulator (CFTR) protein. These nonsense “class I” mutations are notoriously hard to treat as no CFTR protein is expressed and the available CFTR modulator and corrector therapies are ineffective⁷. PTC therapeutic strategies targeting mRNA degradation and the transcriptional and translational processes involved in the production of CFTR protein will be beneficial.

To that end, a nonsense suppression platform of Anti-Codon Edited (ACE)-tRNAs has been developed. This ACE-tRNA platform has significant advantages over other PTC readthrough strategies as it results in seamless, codon specific PTC suppression. The safety of this platform as a therapeutic is supported by ribosomal profiling studies that indicate that ACE-tRNAs display minimal readthrough of endogenous translation termination codons, suggesting a low proteotoxic burden. To deliver tRNAs to cell types of interest, viral transduction, DNA electroporation, ribonucleoprotein complex, and nanoparticle complex formulation methods may be used. But, while all of these methods show promise, they depend, at least in part, on the efficiency of the DNA or RNA cargo to suppress PTCs.

### SUMMARY

tRNA sequences and expression cassettes that display increased efficiency of PTC suppression have been developed. Such sequences and expression cassettes can address the issues mentioned above in a number of aspects.

In one aspect, the disclosure provides a nucleic acid comprising: (i) nucleotides 1 to 55 of a modified sequence selected from SEQ ID NOs: 1-39 and 41-79 as shown in Table 1A or 1B or (ii) one selected from SEQ ID NOs: 81-90 as shown in Table 1C; and a coding segment encoding a transfer RNA (tRNA); and, optionally, a 3′ trailer segment.

In a second aspect, the disclosure provides a nucleic acid comprising: an optional 5′ leader segment; a coding segment encoding a tRNA; and a 3′ trailer segment comprising nucleotides 129 to 132 of a modified sequence selected from SEQ ID NOs: 92-95 and 97-106 as shown in Table 2A or 2B.

In a third aspect, the disclosure provides a nucleic acid comprising: a 5′ leader segment comprising (i) nucleotides 1 to 55 of a modified sequence selected from SEQ ID NOs: 1-39 and 41-79 shown in Table 1A or 1B or (ii) one selected from SEQ ID NOs: 81-90 as shown in Table 1C; a coding segment encoding a tRNA; and a 3′ trailer segment comprising nucleotides 129 to 132 of a modified sequence selected from SEQ ID NOs: 92-95 and 97-106 as shown in Table 2A or 2B.

In a fourth aspect, the disclosure provides a nucleic acid comprising any one of the sequences provided herein or an optimized portion of any one of the sequences provided herein alone or in combination with sequence encoding a tRNA.

In general, the nucleic acid may be at least 120 nucleotides (nt) (e.g., about 120 to about 500 nt, about 130 to about 400 nt, about 140 to about 300 nt, and about 150 to about 200 nt) in length. At least two of the 5′ leader segment, the coding segment, and the 3′ trailer segment are heterologous to one another or not from the same gene in some embodiment. In some embodiments, the nucleic acid described above may comprise a modified sequence selected from SEQ ID NOs: 1-39, 41-79, 81-90, 92-95, 97-106, 152, 153-162, 164-177, 179-194, 196-210, and 228-251 as shown in Table 1A, 1B, 1C, 2A, 2B, 9, or 10. In some embodiments, the nucleic further comprises a tabulator sequence. The tabulator sequence can comprise any suitable sequence. In one embodiment, the tabulator sequence encodes a ribozyme. In one embodiment, the tabulator sequence encodes a self-cleaving ribozyme.

In other embodiments, the nucleic acid described above may comprise nucleotides 56-128 of a modified sequence selected from SEQ ID NOs: 108-111, 113-117, 118-131, and 133-146 as shown in one of Tables 3, 4, 5, 6, and 7. For example, the nucleic acid may comprise a modified sequence shown in one of Tables 3, 4, 5, 6, and 7.

Each of the nucleic acids described above may further comprise an upstream control element (UCE) operably linked to the 5′ end of a 5′ leader segment. In some embodiments, the upstream control element comprises a U6 promoter or an H1 promoter (such as those in Table 8) or a sequence selected from SEQ ID NOs: 148-151. In each of the nucleic acids described above, the tRNA may be an anti-codon edited-tRNA (ACE-tRNA).

The disclosure also provides an expression cassette or an expression vector comprising any one of the nucleic acids provided herein. Also provided here is a host cell comprising the nucleic acid, or the expression cassette, or the expression vector. Also provided is one or more progenies of the host cell. Further provided is a tRNA encoded by the nucleic acid described above.

The nucleic acid can be used in a method for expressing a tRNA, such as an ACE-tRNA, in a cell.

The expressed tRNA can have the function of reverting a PTC to an amino acid during the translation of a mRNA. To that end, the method includes (i) contacting a cell of interest with the nucleic acid molecule described above and (ii) maintaining the cell under conditions permitting expression of the tRNA. The cell can have a mutant nucleic acid comprising one or more PTCs. In that case, the wildtype nucleic acid encodes a fully functional polypeptide. Using the method, the expressed tRNA rescues the one or more PTCs so as to restore expression of the polypeptide or improve the functional activities of the polypeptide in the cell. For instance, the polypeptide can be cystic fibrosis transmembrane conductance regulator (CFTR) and the mutant nucleic acid encodes a truncated CFTR. In one example, the mutant nucleic acid has a Trp-to-Stop PTC. The tRNA translates the Trp-to-Stop PTC into a Leu.

Within the scope of this invention is a host cell comprising one or more of the nucleic acids described herein.

The nucleic acid described above can be used in a method for treating disorders, such as PTC-associated disorders. Accordingly, the invention also provides a pharmaceutical formulation comprising (i) a nucleic acid or expression cassette or expression vector or tRNA as provided herein, and (ii) a pharmaceutically acceptable carrier.

Also provided is a method of treating a disease, such as one associated with a PTC, in a subject in need thereof. The method includes administering to the subject the nucleic acid or expression cassette or expression vector or tRNA as provided herein or the pharmaceutical composition described above.

Examples of the disease include cystic fibrosis, Duchenne and Becker muscular dystrophies, retinoblastoma, neurofibromatosis, ataxia-telangiectasia, Tay-Sachs disease, Wilm's tumor, hemophilia A, hemophilia B, Menkes disease, Ullrich's disease, β-Thalassemia, type 2A and type 3 von Willebrand disease, Robinow syndrome, brachydactyly type B (shortening of digits and metacarpals), inherited susceptibility to mycobacterial infection, inherited retinal disease, inherited bleeding tendency, inherited blindness, congenital neurosensory deafness and colonic agangliosis and inherited neural develop-mental defect including neurosensory deafness, colonic agangliosis, peripheral neuropathy and central dysmyelinating leukodystrophy, Liddle's syndrome, xeroderma pigmentosum, Fanconi's anemia, anemia, hypothyroidism, p53-associated cancers, esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes rickets, Hurler Syndrome, Dravet Syndrome, Spinal Muscular Dystrophy, Usher Syndrome, Aniridia, Choroideremia, Ocular Coloboma, Retinitis pigmentosa, dystrophic epidermolysis bullosa, Pseudoxanthoma elasticum, Alagille Snydrome, Waardenburg-Shah, infantile neuronal ceroid lipofuscinosis, Cystinosis, X-linked nephrogenic diabetes insipidus, and Polycystic kidney disease. In some example, the disease is an eye disease selected from the group consisting of cone dystrophies, Stargardt's disease (STGD1), cone-rod dystrophy, retinitis pigmentosa (RP), increased susceptibility to age-related macular degeneration, Congenital stationary night blindness 2 (CSNB2), Congenital stationary night blindness 1 (CSNB1), Best Disease, VMD, and Leber congenital amaurosis (LCA16). The treatment method can be carried out using any suitable methods, including viral delivery (e.g., lentivirus, poxviruses, herpes simplex virus I, adenoviruses, adeno-associated viruses, and the like), nanoparticles, electroporation, polyethylenimine (PEI), receptor-targeted polyplexes, liposomes, or hydrodynamic injection.

The details of one or more embodiments of the disclosure are set forth in the description below. Other features, objectives, and advantages of the disclosure will be apparent from the description and from the claims.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIGS. 1A, 1B, and 1C are diagrams showing expression and processing of human tRNAs. (FIG. 1A) Diagram of human tRNA gene with 5′-upstream control element (5′ UCE) containing a degenerate, weak TATA box region (TATA), transcription start site (TSS), mature tRNA sequence containing A- and B-box promoter sequences, the anticodon, introns and variable loops, and a 3′-trailer followed by a poly-thymidine (pT) transcription terminator. (FIG. 1B) Human tRNA transcription begins with transcription factor III C (TFIIIC) binding to interal A-and B-box regions of the tRNA gene. TFIIIC recruits transcription factor III B (TFIIIB), which is composed of B-related factor (BRF1), B double prime 1 (BDP1), and the TATA box-binding protein (TBP). TFIIIB recruits RNA polymerase III (Pol III) for transcription initiation. The 3′-trailer and transcription terminator region is also thought to be important for transcription as the transcription apparatus is known to undergo multiple rounds of tRNA transcription following initial assembly, which depends on the tRNA gene 3′-region. (FIG. 1C) A pre-tRNA depicted as a cloverleaf diagram.

FIGS. 2A, 2B, and 2C show maps of ACE-tRNA plasmids used in screening assays for (FIG. 2A) 5′ upstream control element, (FIG. 2B) t-stem and sticky stem mutants, and (FIG. 2C) 3′ trailers.

FIGS. 3A 3B, and 3C show tRNA T-stem mutants and numbering. (FIG. 3A) T-stem variants covering a range of putative tRNA-EF1a interaction strengths to optimize ACE-tRNA function. For the ArgTGA, LeuTGA, and GlyTGA libraries the T-stems of the respective tRNAs were mutated to match the variants as outlined in the table shown here. Sequence variants were adapted from Saks M E et al., RNA. 2011; 17 (6): 1038-1047. (FIG. 3B) All tRNA T-stems are numbered as shown in a cloverleaf T-stem structure with canonical numbering scheme. (FIG. 3C) Suppression efficiency of ACE-tRNA^{Leu}_{UGA} T-stem mutants in 16HBE14o− cells.

FIG. 4 shows an ArgTGA ACE-tRNA cloverleaf structure with “sticky stem” mutation sites noted. The original ArgTGA (oArgTGA) sequence (GGCUCUGGUGGCGCAAUGGAUAGCGCAUUGGACUUCAAAUUCAAAGGUUGUGGGUUCGAGU CCCACCAGAGUCG, SEQ ID NO: 264) is denoted in the cloverleaf structure with base pair mutations shown with arrows indicating the location of G-C or C-G mutations. The sticky stems library contains every combination of either the oArgTGA base pair or the G-C/C-G mutation noted.

FIG. 5 shows a LeuTGA ACE-tRNA cloverleaf structure with “sticky stem” mutation sites noted. The original LeuTGA (oLeuTGA) sequence (ACCAGAAUGGCCGAGUGGUUAAGGCGUUGGACUUCAGAUCCAAUGGAUUCAUAUCCGCGUGG GUUCGAACCCACUUCUGGUA, SEQ ID NO: 265) is denoted in the cloverleaf structure with base pair mutations shown with arrows indicating the location of G-C or C-G mutations. The sticky stems library contains every combination of either the oLeuTGA base pair or the G-C/C-G mutation noted.

FIG. 6 shows types of RNA Polymerase III promoters. Type II RNA Pol III promoters are employed to expressed tRNA genes in humans. Intragenic box A and box B sequences recruit TFIIIC, which then recruits TFIIIB, which then recruits RNA Pol III (FIG. 1B). Type III RNA Pol III promoters express other genes including U6 and H1 RNAs. Type III promoters do not require any intragenic sequences and as such are often used to express exogenous RNAs including CRISPR guide RNAs, siRNAs, and shRNAs which do not contain native A or B boxes. Type III promoters have been used to express human nonsense suppressor tRNAs although many of the endogenous human 55 bp 5′-leaders yield higher nonsense suppression activity in vivo.

FIGS. 7A, 7B, and 7C show assembly of a 55-bp 5′ leader library. (FIG. 7A) A parent high-throughput-cloning and -screening vector for the tRNA 5′ region, contains a cloning site immediately 5′ to the original ArgTGA tRNA sequence/the original 3′ trailer sequence. (FIG. 7B) Human tRNAs denoted with a name such as “tRNA-Ala-AGC-1-1” (where Ala is the three-letter amino acid code for the tRNA isotype, AGC is the anticodon, the first 1 corresponds to the numeric ID of a unique tRNA transcript or “isodecoder”, and the second 1 corresponds to the gene locus ID—for tRNAs that have multiple identical copies, this gene locus ID represents the particular gene copy in the genome). (FIG. 7C) Cloning of these different 55 bp 5′ upstream control elements.

FIGS. 8A, 8B, 8C, 8D, 8E, 8F, and 8G show that appending a tRNA transcript tabulator to the 3′ end of ACE-tRNAs results in ACE-tRNA-dependent expression of an RNA target for quantification by RT-qPCR.

FIG. 8A shows a tRNA transcript tabulator (TTT) within the context of a tRNA gene.

FIG. 8B shows uses of a self-cleaving ribozyme as the TTT (cleavage site denoted with an arrow).

FIG. 8C shows that, while ACE-tRNAs are not amenable to direct quantification via real-time reverse transcription quantitative PCR (RT-qPCR) without extensive processing as they contain many modified nucleotides which inhibit reverse transcriptase, the TTT does not contain modifications which inhibit reverse transcriptase.

FIGS. 8D, 8E, 8F, and 8G show that the TTT abundance is directly proportional to the ACE-tRNA abundance and quantification of the tRNA transcript counter acts as a surrogate for quantification of each ACE-tRNA sequence tested: ACE-tRNA^{Arg}_{TGA} (FIG. 8D), ACE-tRNA^{Leu}_{TGA} (FIG. 8E), ACE-tRNA^{Gly}_{TGA} (FIG. 8F), and ACE-tRNA^{Trp}_{TGA} (FIG. 8G).

FIGS. 9A, 9B, 9C, 9D, and 9E show that optimized ACE-tRNA expression cassettes demonstrate higher peak levels of PTC suppression (higher Supₘₐₓ) and/or display lower levels of DNA delivery needed to reach maximal PTC suppression (lower DD₅₀).

FIG. 9F shows a table of fit data for plots displayed in FIGS. 9A, 9B, 9C, and 9D where curves were fit with the equation shown in FIG. 9E.

FIGS. 10A, 10B, 10C, 10D, and 10E show that optimized ACE-tRNAs retain their fidelity in translational.

FIG. 10A shows that a pcDNA3.1 (+) plasmid encoding superfolder green fluorescent protein (sfGFP) with a TGA stop codon at amino acid position 150 and a C-terminal Strep-8xHistidine-Strep tag was co-transfected with a plasmid encoding 4 copies of the ACE-tRNA under investigation into HEK293T cells.

FIGS. 10B and 10C show that eluted sfGFP proteins were resolved on a 10-20% gradient SDS-PAGE gel and stained with SimplyBlue Safestain (Thermo Fisher Scientific) Coomassie stain.

FIG. 10D shows that the mass of each peptide which contained the amino acid at site 150 in sfGFP was determined and the amino acid with the most consistent mass for position 150 in that peptide was determined. As trypsin cleaves after arginine and lysine, the peptide for arginine at position 150 (LEYNFNSHR, SEQ ID NO: 252 is different than for the other amino acids shown here (LEYNFNSHXVYITADK, SEQ ID NO: 253). Arginine in the peptide denoted as R, other amino acids denoted as X.

FIG. 10E shows the peptide abundance of each amino acid incorporated at position 150 as tallied and expressed as percentages, indicating that translational fidelity was largely retained following optimization of the ACE-tRNA sequences.

FIG. 10F shows a table exhibiting the percentage of each amino acid incorporated at position 150 in sfGFP-TGA-150 as determined by mass spectrometry and shown in FIG. 10E.

FIGS. 11A, 11B, and 11C show determination of the applicability of the best 5′-UCE, 3′-trailer, and t-stem sequences to the improvement of all ACE-tRNAs.

FIG. 11A shows that the top ACE-tRNA sequences for each isoacceptor/stop codon family from Lueck et al., Nature communications 10, 822, 2019 were cloned with the original 5′-UCE, 3′-trailer, and t-stem were cloned for reference to the improved sequences.

FIG. 11B shows that to determine the applicability of the top 5′-UCE and 3′-trailer sequences to improving all ACE-tRNA sequences, each top ACE-tRNA sequence for each isoacceptor/stop codon family from Lueck et al., 2019, was cloned flanked by the top 5′-UCE and 3′-trailer sequences determined in this study.

FIG. 11C shows that to determine the applicability of the top t-stem sequence to improving all ACE-tRNA sequences, each top ACE-tRNA sequence for each isoacceptor/stop codon family from Lueck et al., 2019, was cloned with the top t-stem sequence determined in this study.

### DETAILED DESCRIPTION OF THE INVENTION

This disclosure relates to RNAs, including tRNAs, such as ACE-tRNAs, expression cassettes or expression vectors, and related delivery and uses for treatment of disorders, such as those associated with PTC or nonsense mutations.

Due to the high prevalence and unifying mechanism of nonsense mutations resulting in disease, there has been a significant effort to identify PTC therapeutics. Natural and synthetic aminoglycosides have been the major focus of these efforts, although they have limited use due to ototoxicity and nephrotoxicity with extended use, or suffer from low read-through efficiency⁸⁻¹¹. Non-aminoglycoside small molecules (i.e. tylosin, Ataluren) have also been identified as promising PTC read-through compounds with little toxicity¹²⁻¹³. A major drawback to all small molecule read-through compounds developed so far is the suppression of PTCs by near-cognate tRNAs often resulting in the conversion of the nonsense mutation to a missense mutation. Non-selective incorporation of an amino acid at the PTC location has the potential to affect protein folding, trafficking, and function, requiring further therapeutic intervention, i.e. promotion of folding and/or trafficking¹⁴. In addition some of these compounds display unexpectedly low efficiency of CFTR PTC suppression in human primary cells¹⁵ and patient populations, resulting in failed phase 3 clinical trials for Ataluren (ACT DMD Phase 3 clinical trial, NCT01826487; ACT CF, NCT02139306). A promising new eukaryotic ribosome selective glycoside lead compound, ELOX-2, is under development by Eloxx Pharmaceuticals¹⁶. However, despite the significant interest there is currently no FDA-approved therapeutic agent for the treatment of CF caused by nonsense mutations.

### ACE-tRNAs

An ACE-tRNA is an engineered tRNA molecule whose sequence is engineered so that a PTC is effectively and therapeutically reverted back into the originally lost amino acid or a different amino acid. Such engineered tRNAs allow for “re-editing” of a disease-causing nonsense codon to a specific amino acid. The small size of these tRNA molecules makes them amenable to ready expression, as the tRNA and the promoter together can be only about 300 bp. To that end, an oligonucleotide can be synthesized to comprise the structural component of a tRNA gene functional in human cells. The sequence of this oligonucleotide can be designed based upon a known sequence with substitutions made in the anticodon region of the tRNA causing the specific tRNA to recognize a nonsense or other specific mutation. Examples of ACE-tRNAs include those described in WO2021252354, WO2019090154, WO2019090169, and Lueck, J. D. et al. Nature communications 10, 822, 2019. The contents of each of these documents, including the ACE-tRNA sequences provided therein, are incorporated by reference.

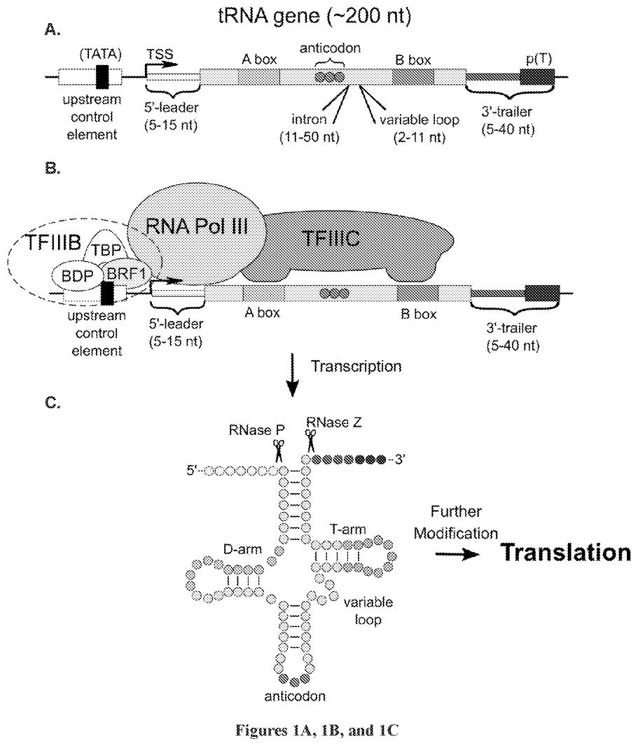

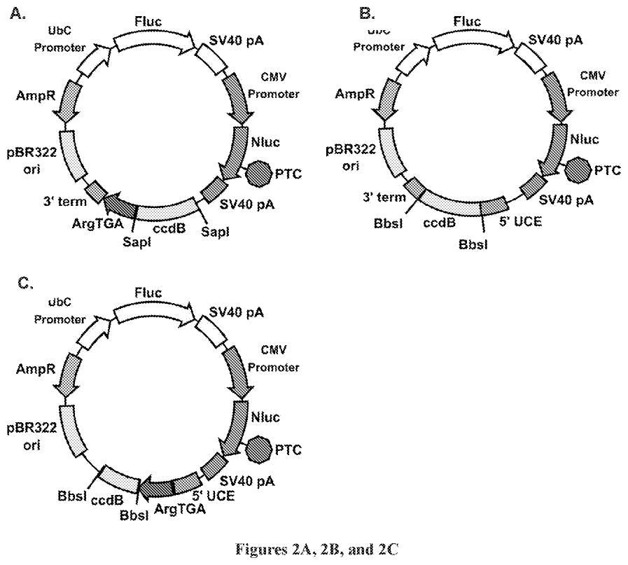

In general, a tRNA has a general four-arm structure comprising a T-arm, a D-arm, and anticodon-arm, and an acceptor arm (see FIG. 1C of this application and FIG. 2 of WO2019090169). The T-arm is made up of a “T-stem” and a “TIC loop.” In certain embodiments, the T-stem is modified to increase the stability of the tRNA. In certain embodiments, the tRNA has a modified T-stem that increases the biological activity to suppress stop sites relative to the endogenous T-stem sequence.

tRNAs, such as ACE-tRNAs, can be used for suppression of PTCs. Yet, efficacious suppression of PTCs has potential drawbacks. For example, there was concern that a PTC suppression strategy could result in readthrough of real, native stop codons in vivo and readthrough of global native stop codons is deleterious. However, several cellular mechanisms are in place to limit both normal stop read-through and damaging effects thereof. More specifically, multiple in-frame stop codons are frequently found at normal translation termination, thus increasing the probability of translation termination in the presence of an efficient PTC suppressor. Furthermore, at least two cellular mechanisms are in place for the identification and degradation of proteins with erroneous translation termination, specialized ubiquitin ligases and ribosome associated pathway. There is evidence that natural stop codons at the end of genes have surrounding sequence landscapes that promote enhanced termination efficiency, and that termination complexes found at PTCs differ from those at “real” stops. Unexpectedly, discovery that endogenous stop codon read-through is common in animals and is not detrimental suggests that suppression of PTCs is a viable therapeutic approach. Indeed, ribosomal profiling preliminary data suggest that tRNA readthrough of “real” stops is infrequent. tRNAs, such as ACE-tRNAs, useful for this disclosure can be made according to the strategy described in WO2021252354, WO2019090154, WO2019090169, and Lueck, J. D. et al., Nature communications 10, 822 (2019). Using this strategy, an extensive library of ACE-tRNAs for effective rescue of PTCs in cell culture have been made.

A novel high throughput screening (HTS) platform was developed for generation of a library of ACE-tRNAs for effective rescue of CFTR PTCs in cell culture. To do this the human tRNA sequences were engineered to no longer suppress their cognate codon, but instead suppress disease-causing PTCs, resulting in full-length functional CFTR protein¹. The anticodon editing strategy has been pursued in the past for suppression of PTCs in β-thalassemia and Duchenne muscular dystrophy¹⁷⁻¹⁸. However, in both of these cases only a single tRNA was targeted for anticodon editing, while the ACE-tRNA screen tested all possible suppressor tRNAs for each PTC. Suppressor tRNAs have also been used extensively in the field of genetic code expansion (GCE) for the incorporation of noncanonical amino acids into proteins. To do so, GCE suppressor tRNAs have been stably expressed in mammalian cells¹⁹, D. melanogaster²⁰, C. elegans²¹, and zebrafish²², without deleterious effects.

The screen was successful in selecting ACE-tRNAs that efficiently promote suppression of the CF-causing PTCs W1282X and R1162X (see Example 1). A study showed that W1282L mutation results in >80% of the WT CFTR activity²³, and the ACE-tRNALeuUGA hit was found to be high performing.

The ultimate goal of investigating these ACE-tRNAs is to show that they are safe and efficacious, such as for CFTR PTC suppression in vivo for the treatment of CF-causing nonsense mutations, and to develop a method for their delivery as therapeutics. While multiple avenues for delivery of ACE-tRNAs including viral transduction, DNA electroporation, ribonucleoprotein complex, and nanoparticle complex formulation methods being pursued, they all can depend on the efficacy of tRNA cargo.

Optimal Sequences for Efficient Transcription and Processing of tRNAs

As disclosed herein, tRNAs, such as ACE-tRNAs, are well-suited for PTC therapeutics. tRNA genes are transcribed into tRNAs by type 2 RNA polymerase (Pol) III recognition of internal promoter elements (A and B boxes, FIG. 1), wherein the tRNAsare flanked by a short (<50 bp) 5′ flanking region and a 3′ transcription termination elements consisting of a short run of thymidine nucleotides (˜4 Thymidines, Ts). Most tRNA genes are 72-76 bps in length, and therefore an entire tRNA expression cassette can consist of only about 125 bps.

In order to give tRNAs the greatest chance to realize their full therapeutic potential, this disclosure provides sequences to optimize already promising tRNA hits to increase their efficacy. As disclosed herein, these optimized sequences can lead to enhanced RNA expression, and in the case of ACE-tRNAs, enhanced nonsense mutation suppression. Examples of the sequences are shown in Tables 1A, 1B, 1C, 2A, 2B, 3 to 7, and 9.

There are a variety of sequence elements involved with production (transcription) and translational function of a tRNA that can be optimized. RNA polymerase III drives expression of tRNA genes in eukaryotes utilizing type 2 intragenic promoter elements (A and B boxes, FIG. 1A)³¹. While the A and B boxes are sufficient for type 2 promoter function the expression of tRNAs are modulated and enhanced by the sequence ˜50 bp immediately 5′ to the gene (5′-flanking sequence)³²⁻³³. Transcription of tRNA is terminated by ashort stretch of thymidine nucleotides (≤4 thymidines, Ts)³¹. Initially a precursor tRNA is synthesized with short 5′-leader and 3′-trailer sequences (FIG. 1B), which are removed by the action of several nucleases. 5′ End processing by the endonuclease RNase P is generally the first step of the tRNA processing pathway³⁴.

The 5′-flanking sequence can be important for the production of active tRNA not only because the 5′-transcriptional modulator affects the amount of tRNA transcribed but also because the 5′ leader sequence affects the efficiency of cleavage by RNase P and the tRNA maturation process³⁵. 3′ end processing of pre-tRNAs generally follows 5′ end processing and is catalyzed by exonucleases³⁶⁻³⁸ and the RNase Z endonuclease³⁹. Both the length and the identity of the first nucleotide of the 3′-trailer affect the 3′ processing efficiency⁴⁰. Shorter 3′-trailers beginning with a G or A are generally processed more efficiently⁴⁰, however no steadfast rules have been established.

As with the 5′-leader, cleavage of the 3′-trailer is important for the maturation of pre-tRNA. Following 3′ end processing a 3′ CCA trinucleotide is ligated and the tRNA is then modified with on average eight (per tRNA) of the >90 known tRNA post-transcriptional modifications (FIG. 1C)⁴¹.

Following the production of a mature tRNA it is recognized by its cognate aminoacyl-tRNA synthetase (aaRS) and charged with the correct amino acid. The ArgRS and the LeuRS both contain an anticodon binding/recognition domain⁴². It is evident from an initial ACE-tRNA screen that converting the anticodon of an ACE-tRNA from the canonical anticodon to a nonsense suppression anticodon disrupts ACE-tRNA/aaRS interactions (FIG. 1C). It is hypothesized that optimizing the anticodon loop can enhance favorable interactions with the aaRS and increase charging of ACE-tRNAs, resulting in increased translational participation of ACE-tRNAs and PTC suppression.

Screening a library of mutants in the anticodon loop of nonsense suppressing tRNAs has been shown to significantly increase the efficiency of nonsense suppression in GCE^{5, 27-28, 30, 43}. After being charged with its cognate tRNA the amino-acylated ACE-tRNA is transported to the ribosome by EF-1a, which recognizes both the charged amino acid and the T stem to promote proper tRNA transport and release to the ribosome⁴⁴⁻⁴⁶. The tRNA then enters the A site of the ribosome, suppresses a PTC, and can allow translation of full-length CFTR protein, for example.

As an example, a pre-tRNA depicted in FIG. 1C as a cloverleaf diagram is the initial product of tRNA transcription containing a 5′-leader, in some cases an intron, and a 3′-trailer. The 5′-leader is removed through the action of RNase P endonuclease, the intron (if present) is spliced out, and the 3′ trailer is removed through the endonuclease action of RNase Z or the action of other exonucleases. Nucleobases are modified through enzymatic activity before the mature tRNA goes on to perform its intracellular function in the translational apparatus. The identity of the tRNA anticodon loop is often important for recognition and charging by the cognate aminoacyl tRNA synthetase (aaRS). As this region must be altered for a functional nonsense suppressor tRNA, this region of the tRNA represents a potential site of functional improvement. Following charging of the tRNA by its cognate aaRS, the aminoacylated tRNA is transported to the ribosome by the elongation factor 1α (EF1a). As EF1a is known to interact with the t-stem or t-arm of the tRNA, this region represents another potential site of improvement of tRNA function. Generally it is thought that the thermodynamic stability of tRNAs is closely related with their intracellular stability and half-life, as such increasing the ratio of G-C to A-T content in the base-paired stems (D-stem, anticodon stem, T-stem, and acceptor stem) and variable loop (if applicable) can increase the stability, lead to longer tRNA half-lives, a higher intracellular pool of tRNAs, and increased nonsense suppression.

### Reference Sequence/Original Sequence

Shown below is an exemplary sequence encoding an ACE-tRNA ArgTGA (i.e., tRNA-Tyr-GTA-5-1) and components thereof, including a 55-bp 5′ leader, a tRNA coding region (in bold), and a 4-bp 3′ trailer region (italic), and RNA Pol. III terminator. The tRNA coding region include an anticodon Loop, an anticodon (underlined), and a T-stem. This sequence is used as a reference sequence or original sequence to describe other mutant or variant sequences ending tRNAs and their various components.

AGCGCTCCGGTTTTTCTGTGCTGAACCTCAGGGGACGCCGACACACGTACACGTCGGCTCTG
TGGCGCAATGGATAGCGCATTGGACTTCAAATTCAAAGGTTGTGGGTTCGAGTCCCACCAGA
GTCGGTCCTTTTTTT (SEQ ID NO: 254)

| | | | SEQ ID |
|---|---|---|---|
| Nucleotides | Components | Sequences | NO |
| 1-55 | 55 bp | AGCGCTCCGGTTTTTCTGTGCTGAACC | 255 |
| | 5′ leader | TCAGGGGACGCCGACACACGTACACGT | |
| | | C | |
| 56-128 | ACE-tRNA | GGCTCTGTGGCGCAATGGATAGCGCAT | 256 |
| | ArgTGA | TGGACTTCAAATTCAAAGGTTGTGGGT | |
| | | TCGAGTCCCACCAGAGTCG | |
| 87-93 | Anticodon | CTTCAAA | 257 |
| | Loop | | |
| 89-91 | Anticodon | TCA | 258 |
| 104-120 | T-stem | GTGGGTTCGAGTCCCAC | 259 |
| 129-132 | 4 bp 3′ | GTCC | 260 |
| | trailer | | |
| 133-139 | RNA Pol. III | TTTTTTT | 261 |
| | terminator | | |

In Tables 2-7 and 9 shown below, components from this reference sequence/original sequence are either underlined (e.g., 5′ leader sequence) or in bold (e.g., 3′ trailer sequence) or denoted with a prefix of “o” for original. In some of these tables, the reference sequence/original sequence is listed at the last one and the others are modified sequences or mutants.

As shown Example 8, the original 5′ leader is comparable to or better than U6 and H1 promoters in enhancing expression of a gene such as a tRNA gene. Accordingly, this 5′ leader segment and the other 5′ leader segments described herein may be used as an enhancing element (e.g., a promoter or an enhancer) to enhance expression of a gene such as a tRNA gene.

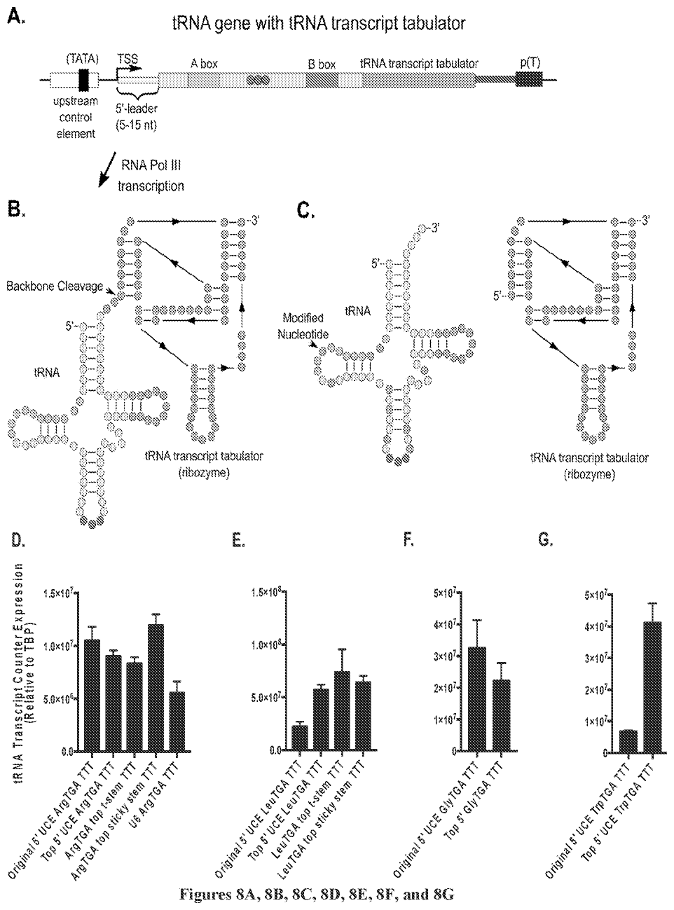

In addition to the components or sequences mentioned above, the tRNA coding sequence can further comprise a tabulator sequence. Such a tabulator sequence is also called “tRNA transcript tabulator” or “tRNA transcript counter.” As disclosed herein (e.g., FIG. 8 and Example 9), the tRNA transcript tabulator allows one to quantify tRNA-dependent expression of an RNA target by suitable means such as RT-qPCR.

The tabulator sequence can comprise any suitable sequence. In one embodiment, the tabulator sequence encodes a ribozyme. In one embodiment, the tabulator sequence encodes a self-cleaving ribozyme. Show below is an exemplary sequence encoding an ArgTGA tRNA transcript with a tabulator sequence. From the 5′ end to the 3′ end, it includes four pieces: 55 bp 5′ leader (Tyr-GTA-5-1), ACE-tRNA ArgTGA (bold), tRNA transcript tabulator (underlined, SEQ ID NO: 262), and RNA Pol. III terminator.

(SEQ ID NO: 263)
AGCGCTCCGGTTTTTCTGTGCTGAACCTCAGGGGACGCCGACAC
ACGTACACGTCGGCTCTGTGGCGCAATGGATAGCGCATTGGACT
TCAAATTCAAAGGTTGTGGGTTCGAGTCCCACCAGAGTCGGGGG
ACCATAGAAGGAGCGTTCTCGTCGCGGTCCCTGTCAGGCTCGTC
CTGCGAATCCTTCTACCACATGCTTTTTTTT

### Expression Cassette and Expression Vectors

The disclosure also provides an expression cassette, comprising or consisting of a nucleic acid as described above. Where such nucleic acid may not already comprise a promoter, the expression cassette may additionally comprise a promoter. Thus, an expression cassette according to the present invention comprises, in 5′ to 3′ direction, a promoter, an upstream control element, a 5′ leader segment (e.g., nucleotides 1 to 55 of a modified sequence shown in Table 1A or 1B), a coding segment encoding a tRNA, a 3′ trailer segment (e.g., nucleotides 129 to 132 of a modified sequence shown in Table 2A or 2B), and optionally a terminator or other elements. The expression cassette can allow for an easy transfer of a target gene into an organism, preferably a cell and preferably a disease cell.

The expression cassette of the present disclosure is preferably comprised in a vector. Thus, the vector of the present disclosure allows to transform a cell with a target gene or a combination of multiple genes while achieving a high expression or activity of the target gene. Correspondingly the disclosure provides a host cell comprising an expression cassette according to the present disclosure or a nucleic acid according to the present disclosure. The nucleic acid may also comprise a promoter or enhancer such as to allow for the expression of the target gene.

Introduction of Nucleic Acid Encoding tRNAs to Cells

Exogenous genetic material (e.g., a nucleic acid, an expression cassette, or an expression vector encoding one or more therapeutic tRNAs) can be introduced into a target cells of interest in vivo by genetic transfer methods, such as transfection or transduction, to provide a genetically modified cell. Various expression vectors (i.e., vehicles for facilitating delivery of exogenous genetic material into a target cell) are known to one of ordinary skill in the art. As used herein, “exogenous genetic material” refers to a nucleic acid or an oligonucleotide, either natural or synthetic, that is not naturally found in the cells; or if it is naturally found in the cells, it is not transcribed or expressed at biologically significant levels by the cells. Thus, “exogenous genetic material” includes, for example, a non-naturally occurring nucleic acid that can be transcribed into a tRNA.

As used herein, “transfection of cells” refers to the acquisition by a cell of new genetic material by incorporation of added nucleic acid (DNA, RNA, or a hybrid thereof). Thus, transfection refers to the introducing of nucleic acid into a cell using physical or chemical methods. Several transfection techniques are known to those of ordinary skill in the art including: calcium phosphate nucleic acid co-precipitation, strontium phosphate nucleic acid co-precipitation, DEAE-dextran, electroporation, cationic liposome-mediated transfection, and tungsten particle-facilitated microparticle bombardment. In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous genetic material contained within the retrovirus is incorporated into the genome of the transduced cell. A cell that has been transduced with a chimeric DNA virus (e.g., an adenovirus carrying a cDNA encoding a therapeutic agent), does not have the exogenous genetic material incorporated into its genome but is capable of expressing the exogenous genetic material that is retained extrachromosomally within the cell.

Typically, the exogenous genetic material may include a heterologous gene (coding for a therapeutic RNA or protein) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion, an “enhancer” is simply any non-translated DNA sequence that works contiguous with the coding sequence (in cis) to change the basal transcription level dictated by the promoter. The exogenous genetic material may introduced into the cell genome immediately downstream from the promoter so that the promoter and coding sequence are operatively linked so as to permit transcription of the coding sequence. A retroviral expression vector may include an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes that encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, ubiquitin, elongation factor-1 and other constitutive promoters known to those of skill in the art. In addition, many viral promoters function constitutively in eucaryotic cells. These include the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. If the gene encoding the therapeutic agent is under the control of an inducible promoter, delivery of the therapeutic agent in situ is triggered by exposing the genetically modified cell in situ to conditions for permitting transcription of the therapeutic agent, e.g., by injection of specific inducers of the inducible promoters which control transcription of the agent. For example, in situ expression by genetically modified cells of a therapeutic agent encoded by a gene under the control of the metallothionein promoter, is enhanced by contacting the genetically modified cells with a solution containing the appropriate (i.e., inducing) metal ions in situ.

Accordingly, the amount of therapeutic agent that is delivered in situ is regulated by controlling such factors as: (1) the nature of the promoter used to direct transcription of the inserted gene, (i.e., whether the promoter is constitutive or inducible, strong or weak); (2) the number of copies of the exogenous gene that are inserted into the cell; (3) the number of transduced/transfected cells that are administered (e.g., implanted) to the patient; (4) the size of the implant (e.g., graft or encapsulated expression system); (5) the number of implants; (6) the length of time the transduced/transfected cells or implants are left in place; and (7) the production rate of the therapeutic agent by the genetically modified cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector may include a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene, and/or signal sequence is deemed to be within the scope of one of ordinary skill in the art without undue experimentation.

A tRNA construct or coding sequence of the present disclosure can be inserted into any type of target or host cell. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Addition of DNA binding proteins such as Transcription Factor A Mitochondria (TFAM) can be used to condense DNA and shield charge. Due to the small size and compact shape, the DNA: Protein (DNP) complexes can then be delivered to cells by cell penetrating peptides, PEG derivative, liposomes or electroporation. In some instances, DNA binding proteins can encode nuclear localization signals to actively transport of DNPs from the cytoplasm to the nucleus where the DNA vectors are transcribed.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

The nucleic acid molecule described herein can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

The minimally invasive electroporation device (“MID”) may be an apparatus for injecting the composition described above and associated fluid into body tissue. The device may comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

The MID may inject the composition into tissue without the use of a needle. The MID may inject the composition as a small stream or jet with such force that the composition pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. patent application No. 20080234655; U.S. Pat. Nos. 6,520,950; 7,171,264; 6,208,893; 6,009,347; 6,120,493; 7,245,963; 7,328,064; and 6,763,264, the contents of each of which are herein incorporated by reference. The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired composition in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the composition into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver compositions to all types of tissues, particularly to skin and mucosa. In some embodiments, a needle-free injector may be used to propel a liquid that contains the composition to the surface and into the subject's skin or mucosa. Representative examples of the various types of tissues that can be treated using the methods disclosed herein include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the tissue. By pulsing between multiple pairs of electrodes in a multiple electrode array, for example set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells may be used. Such a device and system is described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle composition injectors that deliver the composition and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the ELGEN 1000 system, described in U.S. Pat. No. 7,328,064, the contents of which are herein incorporated by reference.

The MID may be a CELLECTRA (INOVIO Pharmaceuticals) device and system, which is a modular electrode system, that facilitates the introduction of a macromolecule, such as a DNA, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra device and system is described in U.S. Pat. No. 7,245,963, the contents of which are herein incorporated by reference. The MID may be an ELGEN 1000 system (INOVIO Pharmaceuticals). The ELGEN 1000 system may comprise device that provides a hollow needle; and fluid delivery means, wherein the apparatus is adapted to actuate the fluid delivery means in use so as to concurrently (for example automatically) inject fluid, the described composition herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue, such as tumor tissue, skin, tissue, liver tissue, and muscle tissue.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached muscle tissue and the apparatus may include means for sensing when the needle has been inserted to a sufficient depth for injection of the fluid to commence. This means that injection of fluid can be prompted to commence automatically when the needle has reached a desired depth (which can normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but can rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor. However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprises means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so users have tended to inject a larger volume of fluid than is required over a larger area and to apply an electric field over a higher area to attempt to guarantee an overlap between the injected substance and the electric field. As described herein, both the volume of fluid injected and the size of electric field applied may be reduced while achieving a good fit between the electric field and the fluid.

Regardless of the method used to introduce exogenous nucleic acids into a host cell, in order to confirm the presence of the recombinant nucleic acid sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or other assays known to those of skill in the art.

### Disease Conditions and Methods of Treatment

Certain embodiments of the present disclosure provide a method of treating a disease or disorder, such as associated with PTCs, in a mammal (such as a human) comprising administering a vector encoding a therapeutic agent (e.g., a tRNA, such as an ACE-tRNA) as described herein to the mammal. Certain embodiments of the present disclosure provide a use of a therapeutic agent or vector encoding a therapeutic agent as described herein to prepare a medicament useful for treating the disease in a mammal.

Diseases or disorders associated with PTCs include, but are not limited to, variants of Duchenne muscular dystrophies and Becker muscular dystrophies due to a PTC in dystrophin, retinoblastoma due to a PTC in RBI, neurofibromatosis due to a PTC in NF1 or NF2, ataxia-telangiectasia due to a PTC in ATM, Tay-Sachs disease due to a PTC in HEXA, cystic fibrosis due to a PTC in CFTR, Wilm's tumor due to a PTC in WT1, hemophilia A due to a PTC in factor VIII, hemophilia B due to a PTC in factor IX, p53-associated cancers due to a PTC in p53, Menkes disease, Ullrich's disease, B-Thalassemia due to a PTC in betaglobin, type 2A and type 3 von Willebrand disease due to a PTC in Willebrand factor, Robinow syndrome, brachydactyly type B (shortening of digits and metacarpals), inherited susceptibility to mycobacterial infection due to a PTC in IFNGR1, inherited retinal disease due to a PTC in CRX, inherited bleeding tendency due to a PTC in Coagulation factor X, inherited blindness due to a PTC in Rhodopsin, congenital neurosensory deafness and colonic agangliosis due to a PTC in SOX10 and inherited neural develop-mental defect including neurosensory deafness, colonic agangliosis, peripheral neuropathy and central dysmyelinating leukodystrophy due to a PTC in SOX 10, Liddle's syndrome, xeroderma pigmentosum, Fanconi's anemia, anemia, hypothyroidism, p53-associated cancers (e.g., p53 squamal cell carcinoma, p53 hepatocellular carcinoma, p53 ovarian carcinoma), esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes and ricketsand many others. The present disclosure in one embodiment includes compositions and methods for treating cystic fibrosis by reversing the effects of mutations present that are associated with nonsense mutations through introduction of the tRNAs of the disclosure. Additional disorders include Hurler Syndrome, Dravet Syndrome, Spinal Muscular Dystrophy, Usher Syndrome, Aniridia, Choroideremia, Ocular Coloboma, Retinitis pigmentosa, dystrophic epidermolysis bullosa, Pseudoxanthoma elasticum, Alagille Snydrome, Waardenburg-Shah, infantile neuronal ceroid lipofuscinosis, Cystinosis, X-linked nephrogenic diabetes insipidus, and Polycystic kidney disease.

Diseases or disorders associated with PTCs that can be treated by the molecules and methods described herein also include a number of eye diseases. Examples of the diseases and genes with the specific mutations include:
Cone dystrophies (Stargardt's disease (STGD1), cone-rod dystrophy, retinitis pigmentosa (RP), and increased susceptibility to age-related macular degeneration): KCNV2 Glu143X; KCNV2 Glu306X; KCNV2 Gln76X; KCNV2 Glu148X; CACNA2D4, Tyr802X; CACNA2D4, Arg628X; RP2, Arg120X; Rho, Ser334X; Rpe65, Arg44X; PDE6A, and Lys455X;
Congenital stationary night blindness 2 (CSNB2): CACNA1F, Arg958X; CACNA1F, and Arg830X;
Congenital stationary night blindness 1 (CSNB1): TRPM1, Gln11X; TRPM1, Lys294X; TRPM1, Arg977X; TRPM1, Ser882X; NYX, and W350X;
Best Disease or BVMD, BEST1, Tyr29X; BEST1, Arg200X; BEST1, and Ser517X;
Leber congenital amaurosis (LCA): KCNJ13, Trp53X; KCNJ13, Arg166X; CEP290, Arg151X; CEP290, Gly1890X; CEP290, Lys1575X; CEP290, Arg1271X; CEP290, Arg1782X; CRB1, Cys1332X; GUCY2D, Ser448X; GUCY2D, Arg41091X; LCA5, Gln279X; RDH12, Tyr194X; RDH12, Glu275X; SPATA7, Arg108X; TULP1, and Gln301X;
Usher syndrome 1: USH1C, Arg31X; PCDH15, Arg3X; PCDH15, Arg245X; PCDH15, Arg643X; PCDH15, Arg929X; IQCB1, Arg461X; IQCB1, Arg489X; PDE6A, Gln69X; ALMS1, Ser999X; ALMS1, and Arg3804X;
Aniridia: Pax6 adn Gly 194X;
Ocular coloboma: Pax2 and Arg139X;
Lamb1, Arg524X;
Choroideremia: REP1 and Gln32X.

According to one aspect, a cell expression system for expressing a therapeutic agent in a mammalian recipient is provided. The expression system (also referred to herein as a “genetically modified cell”) comprises a cell and an expression vector for expressing the therapeutic agent. Expression vectors include, but are not limited to, viruses, plasmids, and other vehicles for delivering heterologous genetic material to cells. Accordingly, the term “expression vector” as used herein refers to a vehicle for delivering heterologous genetic material to a cell. In particular, the expression vector can be a CEDT or MC minivector as described in WO2021252354. Other examples of the expression vector include a recombinant adenoviral, adeno-associated virus, or lentivirus or retrovirus vector.

The expression vector further includes a promoter for controlling transcription of the heterologous gene. The promoter may be an inducible promoter. The expression system is suitable for administration to the mammalian recipient. The expression system may comprise a plurality of non-immortalized genetically modified cells, each cell containing at least one gene encoding at least one therapeutic agent.

The cell expression system can be formed in vivo. According to yet another aspect, a method for treating a mammalian recipient in vivo is provided. The method includes introducing an expression vector for expressing a heterologous gene product into a cell of the patient in situ, such as via intravenous administration. To form the expression system in vivo, an expression vector for expressing the therapeutic agent is introduced in vivo into the mammalian recipient i.v.

According to yet another aspect, a method for treating a mammalian recipient in vivo is provided. The method includes introducing the target therapeutic agent into the patient in vivo. The expression vector for expressing the heterologous gene may include an inducible promoter for controlling transcription of the heterologous gene product. Accordingly, delivery of the therapeutic agent in situ is controlled by exposing the cell in situ to conditions, which induce transcription of the heterologous gene.

The present disclosure provides methods of treating a disease in a subject (e.g., a mammal) by administering an expression vector encoding a tRNA, such as an ACE-tRNA, to a cell or patient. For the gene therapy methods, a person having ordinary skill in the art of molecular biology and gene therapy would be able to determine, without undue experimentation, the appropriate dosages and routes of administration of the expression vector used in the novel methods of the present disclosure.

In certain embodiments, the agents and methods described herein can be used for the treatment/management of diseases, such as those caused by PTCs. Examples include, but are not limited to, Duchenne and Becker muscular dystrophies, retinoblastoma, neurofibromatosis, ataxia-telangiectasia, Tay-Sachs disease, cystic fibrosis, Wilm's tumor, hemophilia A, hemophilia B, Menkes disease, Ullrich's disease, β-Thalassemia, type 2A and type 3 von Willebrand disease, Robinow syndrome, brachydactyly type B (shortening of digits and metacarpals), inherited susceptibility to mycobacterial infection, inherited retinal disease, inherited bleeding tendency, inherited blindness, congenital neurosensory deafness and colonic agangliosis and inherited neural develop-mental defect including neurosensory deafness, colonic agangliosis, peripheral neuropathy and central dysmyelinating leukodystrophy, Liddle's syndrome, xeroderma pigmentosum, Fanconi's anemia, anemia, hypothyroidism, p53-associated cancers (e.g., p53 squamal cell carcinoma, p53 hepatocellular carcinoma, p53 ovarian carcinoma), esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes and rickets. This therapy is advantageous in that it provides improved stop codon suppression specificity. The therapeutic tRNAs of the present disclosure can target a specific stop-codon, TGA for instance, thus reducing off-target effects at stop-codons unrelated to disease, in some embodiments. The present therapy is also advantageous in that it provides amino-acid specificity. The expressed tRNA can be engineered to specifically replace the amino acid that was lost via insertion of a disease stop codon, thus negating any spurious effects on protein stability, folding and trafficking, in some embodiments.

In certain embodiments, the present system is modular, and thus can be “personalized” to every possible disease, such as a PTC disease. For instance, there are nine individual tryptophan tRNAs in the human genome that are recognized by the Trp synthetase, all of which suppress the mRNA UGG codon. Thus, each of these nine Trp tRNA provides an opportunity for codon re-editing tolerance (UGG→UGA). Additionally, given their proximity to stop codons in the genetic code, the mutation of arginine codons to PTC nonsense codons are common in disease. There are over thirty Arg tRNAs that could be tested for codon editing tolerance and suppression efficacy. A tRNA that encodes and Arginine is a viable therapeutic for all Arg->PTC mutations regardless of gene. Indeed, 35% of LCA is caused by nonsense mutations and the majority of those are Arginine to stops. A further advantage of the present disclosure is that it can provide facile expression and cell specific delivery as the entire system (tRNA+promoter sequence) is compact.

### Formulations

Once the cassette, vector, or another form of a therapeutic produced in accordance with this disclosure has been generated and purified in a sufficient quantity, a process of the disclosure may further comprise its formulation, for example as a therapeutic DNA composition. A therapeutic DNA composition comprises a therapeutic DNA molecule encoding a tRNA as provided herein. Such a composition can comprise a therapeutically effective amount of the DNA in a form suitable for administration by a desired route e.g., an aerosol, an injectable composition or a formulation suitable for oral, mucosal or topical administration. Formulation of DNA as a conventional pharmaceutical preparation may be done using standard pharmaceutical formulation chemistries and methodologies, which are available to those skilled in the art.

Any pharmaceutically acceptable carrier or excipient may be used. Auxiliary substances, such as wetting or emulsifying agents, pH buffering substances and the like, may be present in the excipient or vehicle. These excipients, vehicles and auxiliary substances are generally pharmaceutical agents which may be administered without undue toxicity and which, in the case of vaccine compositions will not induce an immune response in the individual receiving the composition. A suitable carrier may be a liposome.

Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. It is also preferred, although not required, that the preparation can contain a pharmaceutically acceptable excipient that serves as a stabilizer, particularly for peptide, protein or other like molecules if they are to be included in the composition. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), and combination thereof. A thorough discussion of pharmaceutically acceptable excipients, vehicles and auxiliary substances is available in REMINGTON'S PHARMACEUTICAL SCIENCES (Mack Pub. Co., N.J. 1991), incorporated herein by reference.

The agents (e.g., a nucleic acid, an expression cassette, or an expression vector) of the disclosure can be administered so as to result in a reduction in at least one symptom associated with a disease (such as a genetic disease, e.g., cystic fibrosis). The amount administered varies depending on various factors including, but not limited to, the composition chosen, the particular disease, the weight, the physical condition, and the age of the subject, and whether prevention or treatment is to be achieved. Such factors can be readily determined by the clinician employing animal models or other test systems that are well known to the art.

The present disclosure envisions treating a disease or disorder by the administration of an agent, e.g., tRNA or an expression vector disclosed in this disclosure. Administration of the therapeutic agents in accordance with the present disclosure may be continuous or intermittent, depending, for example, upon the recipient's physiological condition, whether the purpose of the administration is therapeutic or prophylactic, and other factors known to skilled practitioners. The administration of the agents of the disclosure may be essentially continuous over a preselected period of time or may be in a series of spaced doses. Both local and systemic administration is contemplated.

One or more suitable unit dosage forms having the therapeutic agent(s) of the disclosure, which, as discussed below, may optionally be formulated for sustained release (for example using microencapsulation), can be administered by a variety of routes including parenteral, including by intravenous and intramuscular routes, as well as by direct injection into the diseased tissue. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the disclosure are prepared for administration, they may be combined with a pharmaceutically acceptable carrier, diluent or excipient to form a pharmaceutical formulation, or unit dosage form. The total active ingredients in such formulations include from 0.1 to 99.9% by weight of the formulation. A pharmaceutically acceptable carrier can be a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the disclosure can be prepared by procedures known in the art using well-known and readily available ingredients. The therapeutic agents of the disclosure can also be formulated as solutions appropriate for parenteral administration, for instance by intramuscular, subcutaneous or intravenous routes. The pharmaceutical formulations of the therapeutic agents of the disclosure can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present disclosure may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present disclosure include water and physiologically acceptable buffered saline solutions such as phosphate buffered saline solutions pH 7.0-8.0 and water

### Nanoparticle Compositions

In some embodiments, the pharmaceutical compositions disclosed herein are formulated as lipid nanoparticles (LNP), such as those described in WO2020263883, WO2013123523, WO2012170930, WO2011127255 and WO2008103276; and US20130171646, each of which is herein incorporated by reference in its entirety. Accordingly, the present disclosure provides nanoparticle compositions comprising (i) a lipid composition comprising a delivery agent, and (ii) at least one nucleic acid, such as a tRNA or a DNA encoding the tRNA, e.g., a cassette or a vector. In such a nanoparticle composition, the lipid composition disclosed herein can encapsulate the nucleic acid.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles, liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle comprises an ionizable lipid, a structural lipid, a phospholipid, and nucleic acid of interest. In some embodiments, the LNP comprises an ionizable lipid, a PEG-modified lipid, a sterol and a structural lipid. In some embodiments, the LNP has a molar ratio of about 20-60% ionizable lipid: about 5-25% structural lipid: about 25-55% sterol; and about 0.5-15% PEG-modified lipid.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids lead them to form liposomes, vesicles, or membranes in aqueous media.

In some embodiments, a lipid nanoparticle may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid.” In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively charged moieties include amine groups (e.g., primary, secondary, and/or tertiary amines), ammonium groups, pyridinium group, guanidine groups, and imidizolium groups. In a particular embodiment, the charged moieties can comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

IN some embodiments, the ionizable lipid is an ionizable amino lipid, sometimes referred to in the art as an “ionizable cationic lipid,” In one embodiment, the ionizable amino lipid may have a positively charged hydrophilic head and a hydrophobic tail that are connected via a linker structure. In addition to these, an ionizable lipid may also be a lipid including a cyclic amine group. In one embodiment, the ionizable lipid may be selected from, but not limited to, an ionizable lipid described in WO2013086354 and WO2013116126; the contents of each of which are herein incorporated by reference in their entirety. In yet another embodiment, the ionizable lipid may be selected from, but not limited to, formula CLI-CLXXXXII of U.S. Pat. No. 7,404,969; each of which is herein incorporated by reference in their entirety.

In one embodiment, the lipid may be a cleavable lipid such as those described in WO2012170889, which is incorporated by reference in its entirety. In one embodiment, the lipid may be synthesized by methods known in the art and/or as described in WO2013086354, the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential. The size of the nanoparticles can help counter biological reactions such as, but not limited to, inflammation, or can increase the biological effect of the polynucleotide. As used herein, “size” or “mean size” in the context of nanoparticle compositions refers to the mean diameter of a nanoparticle.

In one embodiment, the nucleic acid described herein can formulated in lipid nanoparticles having a diameter from about 10 to about 100 nm. In one embodiment, the nanoparticles have a diameter from about 10 to 500 nm. In one embodiment, the nanoparticle has a diameter greater than 100 nm. In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The term “encapsulation efficiency” of a nucleic acid/polynucleotide describes the amount of the nucleic acid/polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement. Encapsulation efficiency is desirably high (e.g., close to 100%). The encapsulation efficiency can be measured, for example, by comparing the amount of the nucleic acid/polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a nucleic acid/polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a nucleic acid/polynucleotide present in a pharmaceutical composition disclosed herein can depend on multiple factors such as the size of the nucleic acid/polynucleotide, desired target and/or application, or other properties of the nanoparticle composition as well as on the properties of the nucleic acid/polynucleotide. For example, the amount of a nucleic acid/polynucleotide useful in a nanoparticle composition can depend on the size (expressed as length, or molecular mass), sequence, and other characteristics of the nucleic acid/polynucleotide. The relative amounts of a nucleic acid/polynucleotide in a nanoparticle composition can also vary. The relative amounts of the lipid composition and the nucleic acid/polynucleotide present in a lipid nanoparticle composition of the present disclosure can be optimized according to considerations of efficacy and tolerability.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. (2015) “Delivery of oligonucleotides with lipid nanoparticles” Adv. Drug Deliv. Rev. 87:68-80; Silva et al. (2015) “Delivery Systems for Biopharmaceuticals. Part I: Nanoparticles and Microparticles” Curr. Pharm. Technol. 16:940-954; Naseri et al. (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

Lipid nanoparticle formulations typically comprise one or more lipids. In some embodiments, the lipid is an ionizable lipid (e.g., an ionizable amino lipid), sometimes referred to in the art as an “ionizable cationic lipid”. In some embodiments, lipid nanoparticle formulations further comprise other components, including a phospholipid, a structural lipid, and a molecule capable of reducing particle aggregation, for example a PEG or PEG-modified lipid.

Exemplary ionizable lipids include, but not limited to, any one of Compounds 1-342 disclosed herein, DLin-MC,-DMA (MC.), DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C2-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), and any combination thereof. Other exemplary ionizable lipids include, (13Z,16Z)-N,N-dimethyl-3-nonyldocosa-13,16-dien-1-amine (L608), (20Z,23Z)-N,N-dimethylnonacosa-20,23-dien-10-amine, (17Z,20Z)-N,N-dimemylhexacosa-17,20-dien-9-amine, (16Z,19Z)-N5N-dimethylpentacosa-16,19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimeihyloctacosa-19,22-dien-9-amine, (18Z,21Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl]pyrrolidine, (20Z)-N,N-dimethylheptacos-20-en-10-amine, (15Z)-N,N-dimethyl eptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N,N-dimethyl-1-[(1S,2R)-2-octylcyclopropyl]hexadecan-8-amine, N,N-dimethyl-[(IR,2S)-2-undecyIcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R-N,N-dimethyl-1-[(9Z,12Z)-octadeca-9,12-dien-1-yloxy]-3-(octyloxy) propan-2-amine, S-N,N-dimethyl-1-[(9Z,12Z)-octadeca-9,12-dien-1-yloxy]-3-(octyloxy) propan-2-amine, 1-{2-[(9Z,12Z)-octadeca-9,12-dien-1-yloxy]-1-[(octyloxy) methyl]ethyl}pyrrolidine, (2S)-N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy) methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy) propan-2-amine; (2S)-N,N-dimethyl-1-[(6Z,9Z,12Z)-octadeca-6,9,12-trien-1-yloxy]-3-(octyloxy) propan-2-amine, (2S)-1-[(11Z,14Z)-icosa-11,14-dien-1-yloxy]-N,N-dimethyl-3-(pentyloxy) propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy) propan-2-amine, 1-[(13Z,16Z)-docosa-13,16-dien-1-yloxy]-N,N-dimethyl-3-(octyloxy) propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy) propan-2-amine, 1-[(9Z)-hexadec-9-en-1-yloxy]-N,N-dimethyl-3-(octyloxy) propan-2-amine, (2R)-N,N-dimethyl-H (1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl methyl}cyclopropyl]octyl}oxy) propan-2-amine, N,N-dimethyl-1-{[8-(2-oclylcyclopropyl) octyl]oxy}-3-(octyloxy) propan-2-amine, and (11E,20Z,23Z)-N,N-dimethylnonacosa-11,20,2-trien-10-amine, and any combination thereof.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin. In some embodiments, the phospholipids are DLPC, DMPC, DOPC, DPPC, DSPC, DUPC, 18:0 Diether PC, DLnPC, DAPC, DHAPC, DOPE, 4ME 16:0 PE, DSPE, DLPE,DLnPE, DAPE, DHAPE, DOPG, and any combination thereof. In some embodiments, the phospholipids are MPPC, MSPC, PMPC, PSPC, SMPC, SPPC, DHAPE, DOPG, and any combination thereof. In some embodiments, the amount of phospholipids (e.g., DSPC) in the lipid composition ranges from about 1 mol % to about 20 mol %.

The structural lipids include sterols and lipids containing sterol moieties. In some embodiments, the structural lipids include cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the amount of the structural lipids (e.g., cholesterol) in the lipid composition ranges from about 20 mol % to about 60 mol %.

The PEG-modified lipids include PEG-modified phosphatidylethanolamine and phosphatidic acid, PEG-ceramide conjugates (e.g., PEG-CerC,4 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG DMPE, PEG-DPPC, or a PEG-DSPE lipid. In some embodiments, the PEG-lipid are 1,2-dimyristoyl-sn-glycerol methoxypolyethylene glycol (PEG-DMG), 1,2-distearoyl-sn-glycero-3-phosphoethanolamine-N-[amino (polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG moiety has a size of about 1000, 2000, 5000, 10,000, 15,000 or 20,000 Daltons. In some embodiments, the amount of PEG-lipid in the lipid composition ranges from about 0 mol % to about 5 mol %.

In some embodiments, the LNP formulations described herein can additionally comprise a permeability enhancer molecule. Non-limiting permeability enhancer molecules are described in US20050222064, herein incorporated by reference in its entirety.

The LNP formulations can further contain a phosphate conjugate. The phosphate conjugate can increase in vivo circulation times and/or increase the targeted delivery of the nanoparticle. Phosphate conjugates can be made by the methods described in, e.g., WO2013033438 or US20130196948. The LNP formulation can also contain a polymer conjugate (e.g., a water-soluble conjugate) as described in, e.g., US20130059360, US20130196948, and US20130072709. Each of the references is herein incorporated by reference in its entirety.

The LNP formulations can comprise a conjugate to enhance the delivery of nanoparticles in a subject. Further, the conjugate can inhibit phagocytic clearance of the nanoparticles in a subject. In some embodiments, the conjugate can be a “self” peptide designed from the human membrane protein CD47 (e.g., the “self” particles described by Rodriguez et al, Science 2013339, 971-975, herein incorporated by reference in its entirety). As shown by Rodriguez et al. the self-peptides delayed macrophage-mediated clearance of nanoparticles which enhanced delivery of the nanoparticles.

The LNP formulations can comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., WO2012109121, which is incorporated by reference in its entirety).

The LNP formulations can be coated with a surfactant or polymer to improve the delivery of the particle. In some embodiments, the LNP can be coated with a hydrophilic coating such as, but not limited to, PEG coatings and/or coatings that have a neutral surface charge as described in US20130183244, which is incorporated by reference in its entirety.

The LNP formulations can be engineered to alter the surface properties of particles so that the lipid nanoparticles can penetrate the mucosal barrier as described in U.S. Pat. No. 8,241,670 or WO2013110028, each of which is herein incorporated by reference in its entirety. The LNP engineered to penetrate mucus can comprise a polymeric material (i.e., a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly (styrenes), polyimides, polysulfones, polyurethanes, polyacetylenes, polyethylenes, polyethyeneimines, polyisocyanates, polyacrylates, polymethacrylates, polyacrylonitriles, and polyarylates.

LNP engineered to penetrate mucus can also include surface altering agents such as, but not limited to, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin b4 dornase alfa, neltenexine, erdosteine) and various DNases including rhDNase. In some embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., WO2013110028, which is incorporated by reference in its entirety.

In some embodiments, the nucleic acids described herein can be formulated for controlled release and/or targeted delivery. As used herein, “controlled release” refers to a pharmaceutical composition or compound release profile that conforms to a particular pattern of release to effect a therapeutic outcome. In one embodiment, the nucleic acids can be encapsulated into a delivery agent described herein and/or known in the art for controlled release and/or targeted delivery. As used herein, the term “encapsulate” means to enclose, surround or encase. As it relates to the formulation of the nucleic acids of the disclosure, encapsulation can be substantial, complete or partial. The term “substantially encapsulated” means that at least greater than 50, 60, 70, 80, 85, 90, 95, 96, 97, 98, 99, or greater than 99% of the pharmaceutical composition or compound of the disclosure can be enclosed, surrounded or encased within the delivery agent. “Partially encapsulation” means that less than 10, 10, 20, 30, 4050 or less of the pharmaceutical composition or compound of the disclosure can be enclosed, surrounded or encased within the delivery agent.

In some embodiments, the nucleic acid composition can be formulated for sustained release. As used herein, “sustained release” refers to a pharmaceutical composition or compound that conforms to a release rate over a specific period of time. The period of time can include, but is not limited to, hours, days, weeks, months and years. As a non-limiting example, the sustained release nanoparticle composition described herein can be formulated as disclosed in WO2010075072, US20100216804, US20110217377, US20120201859 and US20130150295, each of which is herein incorporated by reference in their entirety. In some embodiments, the nanoparticle composition can be formulated to be target specific, such as those described in WO2008121949, WO2010005726, WO2010005725, WO2011084521 WO2011084518, US20100069426, US20120004293 and US20100104655, each of which is herein incorporated by reference in its entirety.

### Administration

The above-described therapeutic agents and compositions can be used for treating, protecting against, and/or preventing a disease in a subject in need thereof by administering one or more composition described herein to the subject.

Such agents and compositions can be administered in dosages and by techniques well known to those skilled in the medical arts taking into consideration such factors as the age, sex, weight, and condition of the particular subject, and the route of administration. The composition dose can be between 1 μg to 10 mg active component/kg body weight/time, and can be 20 μg to 10 mg component/kg body weight/time.

The agent or composition can be administered prophylactically or therapeutically. In therapeutic applications, the agents or compositions are administered to a subject in need thereof in an amount sufficient to elicit a therapeutic effect. An amount adequate to accomplish this is defined as “therapeutically effective dose.” Amounts effective for this use will depend on, e.g., the particular composition of the composition regimen administered, the manner of administration, the stage and severity of the disease, the general state of health of the subject, and the judgment of the prescribing physician.

The agent or composition can be administered by methods well known in the art as described in Donnelly et al. (Ann. Rev. Immunol. 15:617-648 (1997)), U.S. Pat. Nos. 5,580,859, 5,703,055, and 5,679,647, the contents of all of which are incorporated herein by reference in their entirety. The nucleic acid, such as DNA, of the composition can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector. The composition can be delivered via a variety of routes. Typical delivery routes include parenteral administration, e.g., intradermal, intramuscular or subcutaneous delivery. Other routes include oral administration, intranasal, and intravaginal routes. For the nucleic acid of the composition, such as DNA in particular, the composition can be delivered to the interstitial spaces of tissues of an individual (U.S. Pat. Nos. 5,580,859 and 5,703,055, the contents of all of which are incorporated herein by reference in their entirety). The composition can also be administered to muscle, or can be administered via intradermal or subcutaneous injections, or transdermally, such as by iontophoresis. Epidermal administration of the composition can also be employed. Epidermal administration can involve mechanically or chemically irritating the outermost layer of epidermis to stimulate an immune response to the irritant (U.S. Pat. No. 5,679,647).

In one embodiment, the composition can be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the composition.

The composition can be a liquid preparation such as a suspension, syrup or elixir. The composition can also be a preparation for parenteral, subcutaneous, intradermal, intramuscular or intravenous administration (e.g., injectable administration), such as a sterile suspension or emulsion.

The composition can be incorporated into liposomes, microspheres or other polymer matrices (U.S. Pat. No. 5,703,055; Gregoriadis, Liposome Technology, Vols. Ito III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The tRNA or nucleic acid molecule encoding the tRNA may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal, intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

The tRNA or nucleic acid molecule encoding the tRNA may be delivered to a mammal by several well-known technologies including injection with and without in vivo electroporation, liposome mediated, nanoparticle facilitated, recombinant vectors such as recombinant adenovirus, recombinant adenovirus associated virus and recombinant vaccinia. The tRNA or nucleic acid molecule encoding the tRNA may be delivered via injection, such as DNA injection, and along with in vivo electroporation.

### Electroporation

Administration of the composition via electroporation may be accomplished using electroporation devices that can be configured to deliver to a desired tissue of a mammal a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system or ELGEN electroporator to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μβ, 20 μβ, 10 or 1 μβ, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the compositions described herein, include those described in U.S. Pat. No. 7,245,963 and US2005/0052630, the contents of which are hereby incorporated by reference in their entirety. Other electroporation devices and electroporation methods known in the art can also be used for facilitating delivery of the compositions. See, e.g., U.S. Pat. Nos. 9,452,285, 7,245,963, 5,273,525, 6,110,161, 6,958,060, 6,939,862, 6,697,669, 7,328,064 and US 2005/0052630.

### Definitions

A nucleic acid or polynucleotide refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA.

A “non-natural” or “engineered” or recombinant nucleic acid refers to a nucleic acid the structure of which is not identical to that of any naturally occurring nucleic acid but it may comprise a fragment of a naturally occurring genomic nucleic acid, in some embodiments. The term therefore covers, for example, (a) a nucleic acid which has the sequence of part of a naturally occurring nucleic acid molecule but is not flanked by both of the sequences that flank that part of the molecule as it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment that does not occur in nature; (d) a recombinant nucleotide sequence that does not occur in nature; or (e) an engineered nucleotide sequence that does not occur in nature. The nucleic acid described above can be used to express a tRNA of this disclosure. For this purpose, one can operatively linked the nucleic acid to suitable regulatory sequences to generate an expression vector.

A “recombinant nucleic acid” is a combination of nucleic acid sequences that are joined together using recombinant technology and procedures used to join together nucleic acid sequences.

The terms “heterologous” DNA molecule and “heterologous” nucleic acid, as used herein, each refer to a molecule that originates from a source foreign to the particular host cell or, if from the same source, is modified from its original form. Thus, a heterologous gene in a host cell includes a gene that is endogenous to the particular host cell but has been modified through, for example, the use of shuffling or recombination. When used to describe two nucleic acid segments, the terms mean that the two nucleic acid segments are not from the same gene or, if form the same gene, one or both of them are modified from the original forms. The terms also include non-naturally occurring multiple copies of a naturally occurring DNA molecule. Thus, the terms refer to a nucleic acid segment that is foreign or heterologous to the cell, or homologous to the cell but in a position within the host cell nucleic acid in which the element is not ordinarily found. Exogenous DNA segments are expressed to yield exogenous RNAs or polypeptides. A “homologous DNA molecule” is a DNA molecule that is naturally associated with a host cell into which it is introduced.

A vector refers to a nucleic acid molecule capable of transporting another nucleic acid to which it has been linked. The vector may or may not be capable of autonomous replication or integrate into a host DNA. Examples of the vector include a plasmid, cosmid, or viral vector. The vector includes a nucleic acid in a form suitable for expression of a nucleic acid of interest in a host cell. Preferably the vector includes one or more regulatory sequences operatively linked to the nucleic acid sequence to be expressed.

A “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein or RNA desired, and the like. The expression vector can be introduced into host cells to produce an RNA or a polypeptide of interest. A promoter is defined as a DNA sequence that directs RNA polymerase to bind to DNA and initiate RNA synthesis. A strong promoter is one which causes RNAs to be initiated at high frequency.

A “promoter” is a nucleotide sequence which initiates and regulates transcription of a polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

“Operably linked” refers to an arrangement of elements wherein the components so described are configured so as to perform their usual function. Thus, a given promoter operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence. Thus, the term “operably linked” is intended to encompass any spacing or orientation of the promoter element and the DNA sequence of interest which allows for initiation of transcription of the DNA sequence of interest upon recognition of the promoter element by a transcription complex.

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. It also may include sequences required for proper translation of the nucleotide sequence. The coding region usually codes for an RNA or protein of interest. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development. In certain embodiments, the promoter is a PGK, CMV, RSV, HI or U6 promoter (Pol II and Pol III promoters).

A “nucleic acid fragment” is a portion of a given nucleic acid molecule. The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%), or even at least 95%, 96%, 97%, 98%, or 99% sequence identity, compared to a reference sequence using one of the alignment programs described using standard parameters.

As used herein, “reference sequence” or “original sequence” is a defined sequence used as a basis for sequence comparison. A reference sequence may be a subset or the entirety of a specified sequence; for example, as a segment of a full length cDNA or gene sequence or isolated nucleic acid sequence.

As used herein “minivector” refers to a mini-sized and circular DNA vector system, e.g., a double stranded circular DNA (e.g., a minicircle) or a closed linear DNA molecule (e.g., CEDT), lacking a bacterial origin of replication and an antibiotic selection gene, and having a size of about 100 bp up to about 5 kbp. It can be obtained, for example, by site-specific recombination of a parent plasmid to eliminate plasmid sequences outside of the recombination sites. It contains, for example, a nucleic acid molecule with merely the transgene expression cassette, including promoter and a nucleic acid sequence of interest, wherein the nucleic acid sequence may be, for example, a tRNA for e.g., suppressing PTCs, and, importantly, no bacterial-originated sequences.

The term “subject” includes human and non-human animals. The preferred subject for treatment is a human. As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc) and a human). In one embodiment, the subject is a human. In another embodiment, the subject is an experimental, non-human animal or animal suitable as a disease model.

A disease or disorder associated with a PTC or nonsense mutation, PTC-associated disease, or PTC-associated disease refers to any conditions caused or characterized by one or more nonsense mutations change an amino acid codon to PTC through a single-nucleotide substitution, resulting in a defective truncated protein.

As used herein, “treating” or “treatment” refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder. The terms “prevent,” “preventing,” “prevention,” “prophylactic treatment” and the like refer to reducing the probability of developing a disorder or condition in a subject, who does not have, but is at risk of or susceptible to developing a disorder or condition. “Ameliorating” generally refers to the reduction in the number or severity of signs or symptoms of a disease or disorder.

The terms “prevent,” “preventing” and “prevention” generally refer to a decrease in the occurrence of disease or disorder in a subject. The prevention may be complete, e.g., the total absence of the disease or disorder in the subject. The prevention may also be partial, such that the occurrence of the disease or disorder in the subject is less than that which would have occurred without embodiments of the present disclosure. “Preventing” a disease generally refers to inhibiting the full development of a disease.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active compound. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate.

The term “about” generally refers to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Recitation of ranges of values herein is merely intended to serve as a shorthand method of referring individually to each separate value falling within the range. Unless otherwise indicated herein, each individual value is incorporated into the specification as if it were individually recited herein.

### EXAMPLES

### Example 1

A library of nonsense suppressor tRNAs (ACE-tRNAs) was developed using a novel high throughput cloning (HTC) and screening method (HTS) method, where the anticodon of the human tRNA genes were engineered to suppress disease causing PTC codons (UAG, UAA, and UGA)¹. To generate the library, the ACE-tRNAgenes were efficiently cloned into the HTC plasmid containing a NanoLuc luciferase (NLuc; Promega, Madison WI) expression cassette with an in-frame PTC. The all-in-one plasmid was then transfected into HEK293 cells and the luminescence quantitatively reporting on PTC suppression was measured in 96-well format¹. While the ACE-tRNA library was generated to target all possible PTCs that are resultant of one nucleotide change from amino acid codons, the hits chosen to optimize in the experiments outlined in this example are ACE-tRNA^{Arg}_{UGA} and ACE-tRNA^{Leu}_{UGA} due to the high prevalence of the R1162X and W1282X mutations in CFTR. Screening the ACE-RNA^{Arg}_{UGA} and ACE-tRNA^{Leu}_{UGA} TRNA families in HEK293 cells revealed a number of hits for each family.

In order to maintain the seamless readthrough of PTCs by the correct amino acid, the fidelity of aaRS-tRNA interaction must be maintained following anticodon editing. To determine if the fidelity of the cognate aaRS-ACE-tRNA interaction has been maintained mass spectrometry was performed to examine amino acid incorporation. The results confirmed the specific suppression of the PTC by its cognate aminoacid for each ACE-tRNA type, resulting in seamless PTC suppression¹.

After determining the identity and specificity of ACE-tRNA hits with the NLuc reporter, assays were carried out to show that ACE-tRNA^{Arg}_{UGA} and ACE-tRNA^{Leu}_{UGA} would suppress the endogenous PTCs encoded in the genomic context of the full CFTR gene in 16HBE14o− cells engineered to include R1162X and W1282X mutations. Using the PiggyBac transposon/transposase system and a novel tRNA multimerization system (PB Donkey) based on the pMuLE platform⁴⁷, 16HBE14o− cells containing either the R1162X or the W1282X mutation stably expressing either 8 copies of the ACE-tRNA^{Arg}_{UGA} or the ACE-tRNA^{Leu}_{UGA} were produced. With these cell lines, both the level of CFTR transcript present and the levelof CFTR function rescued were determined.

Nonsense mediated decay (NMD) is a surveillance system in eukaryotic cells that degrades mRNAs containing PTCs⁴⁸. It is hypothesized that efficient expression of ACE-RNA hits increase CFTR transcript expression containing PTCs by inhibiting NMD. Gene specific qRTPCR was performed on mRNA isolated from W1282X cells stably expressing 8 copies of their respective ACE-tRNA or containing an empty PB Donkey insert. It was found that stable expression of only 8x ACE-tRNALeuUGA resulted in a significant increase in steady state CFTR mRNA levels in W1282X 16HBE14o− cells, while expression of ACE-tRNA^{Trp} had limited effect. Using chamber measurements were also performed to show the functional rescue of CFTR function following stable expression of only 8 x ACE-tRNA^{Arg}_{UGA} in R1162X 16HBEo-cells.

It was surprising to see rescue of CFTR function following addition of forskolin and IBMX with no measurable CFTR function in the cells containing an empty PB Donkey insert. Results demonstrate that ACE-tRNAs expressed at low levels can inhibit NMD and promote functional rescue of CFTR expression in the endogenous CFTR genomic context. To lower the hurdle associated with the delivery of ACE-tRNAs, assays were carried out to optimize the transcriptional and translational elements associated with their in vivo function.

### Example 2

With these ACE-tRNA hits therapeutic leads can be developed, such as by further increasing their efficiency of production, maturation, and/or translational activity in vivo.

As the 5′-flanking sequence is important to PTC suppression efficiency following expression of ACE-tRNA from DNA in vivo²⁴, an ACE-tRNA DNA expression cassette including 55 bp of the 5′-flanking sequence from the human tRNA^{Tyr} gene was generated. It was found that removal of the flanking sequence results in lowered nonsense suppression activity for PTC-containing CFTR, highlighting the importance of this transcriptional element for efficient PTC suppression in vivo. It is hypothesized that human tRNA flanking sequences can result in higher ACE-tRNA suppression efficiencies either due to increased positive modulation of tRNA transcription or by increased 5′ end processing by RNase P.

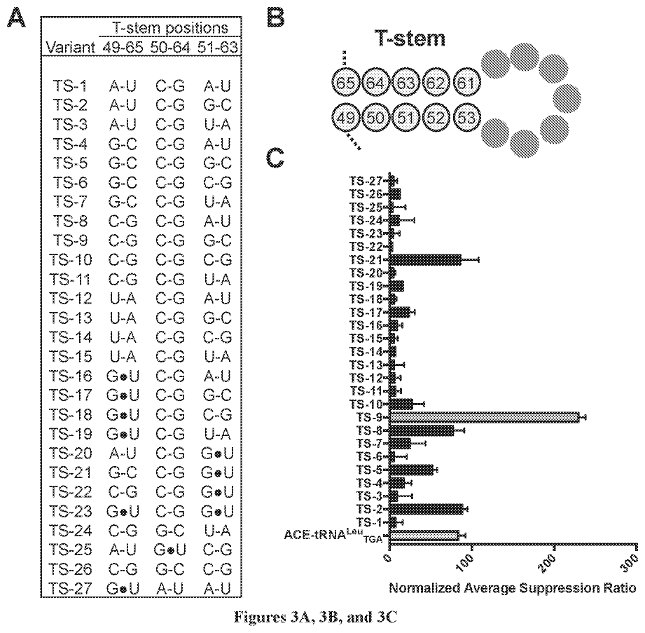

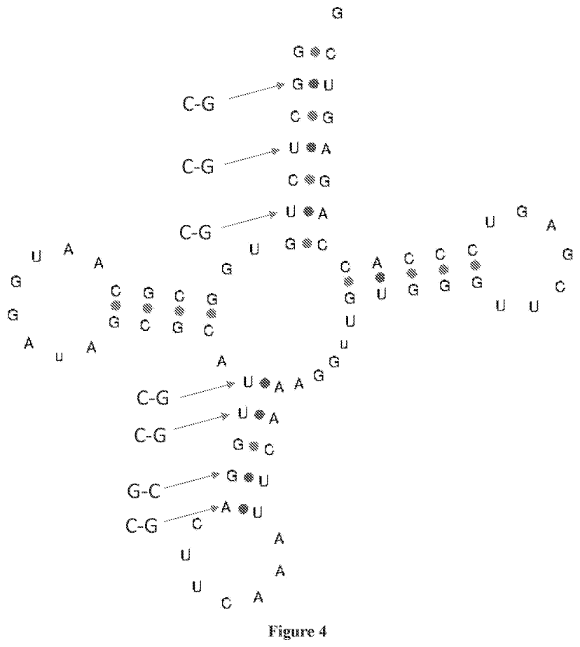

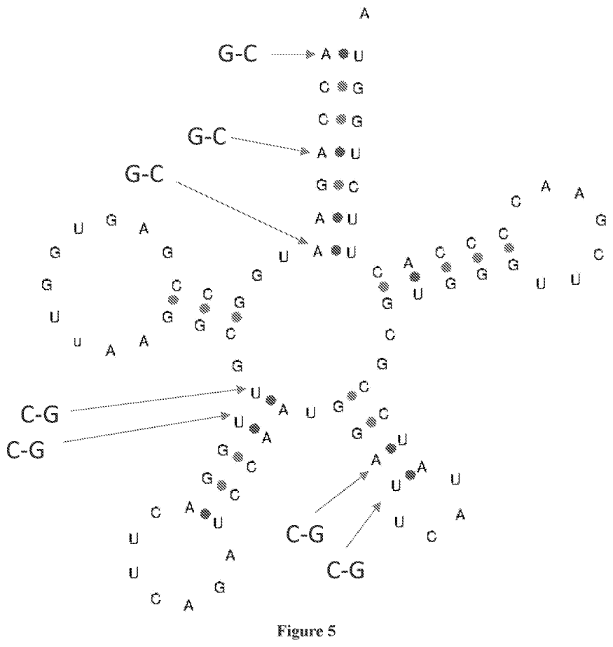

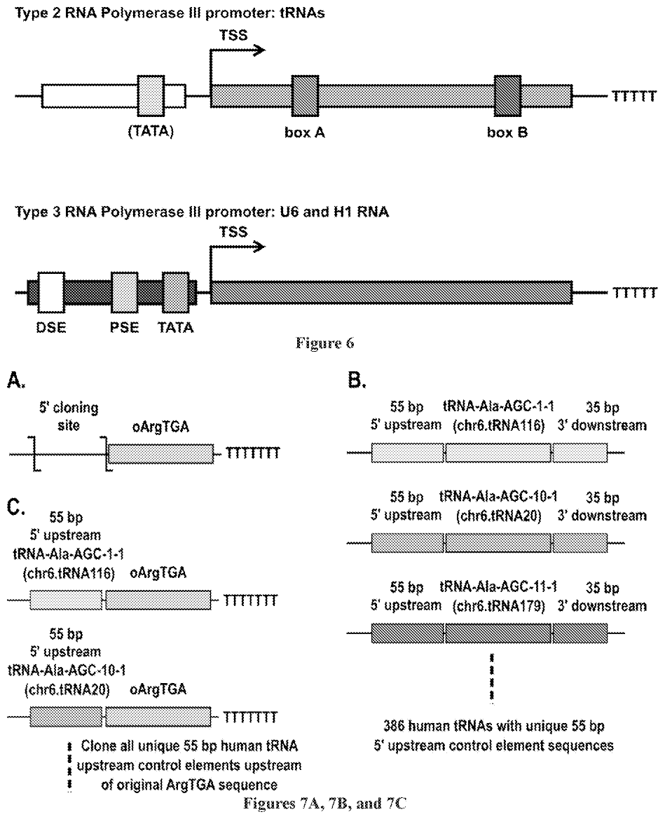

Experiments were carried out to increase the efficiency of ACE-tRNA translational elements. As previously described, the T-stem has been shown to be important for efficient transport and release of aminoacylated tRNAs to the ribosome by the EF-1a homolog EF-Tu⁴⁴⁻⁴⁶. The ACE-tRNA^{Trp}_{UGA} is a poor performing ACE-tRNA amino acid family and in an effort to increase the translational efficiency, the T-stem of the best ACE-tRNA^{Trp}_{UGA} hit was altered. Studies with misacylated bacterial tRNAs show that the EF-1a homolog EF-Tu shows substantial affinity both for the esterified amino acid and the tRNA body^{44, 49-50}. Biochemical experiments using mutations in the base pairs of the T-stem of yeast tRNA^{Phe} revealed that the sequence of the 49-65, 50-64, 51-63 base pairs in the T-stem (see FIG. 3B for numbering) were largely responsible for the sequence-specific binding of this tRNA to the prokaryotic EF-1a homolog EF-Tu⁵¹⁻⁵². A set of single and multiple substitutions of the 49-65, 50-64, and 51-63 base pairs in the T stem of ACE-tRNA^{Trp}_{UGA} (FIG. 3A) were chosen for further screening. These mutations provide a broad range of EF-Tu affinities for each tRNA in E. coli45. The library of ACE-tRNA^{Trp} T-stem mutants was cloned into the same HTC plasmid used for the original screen. Screening the resulting mutants in HEK293 cells revealed several T-stem mutants that increased nonsense suppression efficiency of the ACE-tRNA^{Trp}_{UGA}.

The addition of the 52-62 C-G pair which has previously been shown to increase tRNA efficiency in E. coli⁴⁵ was also explored. It was unexpectedly found that addition of this mutation to the best performing initial mutants (such as TS-21, TS-9; FIGS. 3A and 3C), resulted in the most efficient ACE-tRNA^{Trp}_{UGA} T-stem mutant, which was ˜3× more efficient than the original ACE-tRNA^{Trp}_{UGA}. Results presented in FIG. 3 demonstrate that increasing transcription and processing and tuning translational elements of ACE-tRNAs can increase their PTC suppression efficiency in vivo.

### Example 3

This example describes experimental designs and methods for determining optimal sequences for efficient transcription and processing of ACE-tRNAs from DNA cassettes. Assays were carried out to determine the optimal sequence for the transcriptional elements in ACE-tRNA DNA cassettes to maximize nonsense suppression efficiency following DNA in vivo. It is hypothesized that increasing the efficacy of each ACE-tRNA expression “unit” by enhancing transcription and processing can reduce the amount of ACE-tRNA required to be therapeutic.

3. 1 Determining Optimal 5′-Flanking Sequence for the ACE-tRNA^{Arg}_{UGA} to Improve the Expression and Processing of ACE-tRNAs In Vivo.

In constructing an initial HTC plasmid, a 5′-flanking sequence from a tRNA^{Tyr} gene²⁴ was used. Inclusion of this 5′-flanking sequence in the ACE-tRNA expression cassette can be important for efficient nonsense suppression. While relatively little is known about the function of the 5′-flanking sequence, especially in humans, it has been shown that elements in the 5′-flanking sequence can be responsible for tissue-specific expression of tRNA genes³² and that the efficiency of RNase P cleavage can depend on the pre-tRNA structure⁵³. By screening 5′-flanking sequences from the most highly expressed tRNAs it was hypothesized that both ACE-tRNA transcription and 5′ end processing in human bronchial cells can be optimized simultaneously.

To produce a library of 5′-flanking sequences, the 1000 bp sequences immediately upstream of 48 of the most highly expressed human tRNA genes are cloned⁵⁴. The 5′-flanking sequences are ordered as double stranded DNA eBlocks (IDT, USA) with SapI type IIs restriction enzyme recognition sequences at either end. SapI was chosen as few 5′-flanking sequences already contain the recognition sequence for this restriction enzyme. A ccdB negative selection cassette flanked by SapI recognition sites replaces location of the 5′-leaderin the original HTC plasmid containing the ACE-tRNA^{Arg}_{UGA} (FIG. 2).

Golden Gate reactions to assemble the 5′-flanking sequence HTS library is performed in 96-well PCR plates, as described previously¹. In a deep welled 96-well plate 1 μL of the Golden Gate reaction is transformed into DH5a chemically competent cells (New England Biolabs, USA), grown in 2 mL of Luria Bertani broth supplemented with 100 μg/mL ampicillin at 37° C. for 20 hours, shaking at 300 rpm. E. coli overnight cultures are miniprepped with the PureLink Pro 96 well purification kit (ThermoFisher, USA) and diluted to 125 ng/μL. The HTS is performed in 16HBE14o− cells following transfection of each 5′-flanking sequence HTS library member using Lipofectamine 3000 in black 96-well cell culture blocks. Luminescence from the Nluc-TGA is used an indicator of ACE-tRNA dependent PTC suppression efficiency. Suppression of PTCs by individual 5′-flanking sequence HTS library members is averaged across three wells for each experiment and all clones are repeated >3 times in this fashion. Each plate also contains 16HBE14o− cells transfected with ‘blank’ HTC plasmids with no ACE-tRNAs to serve as a control for transfection efficiency and baseline PTC readthrough. All values are compared as ratios of ACE-tRNA luminescence over baseline nonsense readthrough luminescence±standard deviation. The most efficient 5′-flanking sequence is the library member with the highest luminescence ratio over baseline.

3.2 Determining Optimal 3′-Trailer Sequences for the ACE-tRNA^{Arg}_{UGA} and ACE-tRNA^{Leu}_{UGA} to Improve their Processing In Vivo

The length and sequence of the 3′-trailer has been shown to affect the processing efficiency of RNase Z and 3′ exonucleases³⁶⁻⁴⁰. As with the 5′-flanking sequence, the sequence of the 3′-trailer used for an initial HTC plasmid was taken from previous work with nonsense suppressor tRNAs used for GCE²⁴. A closer inspection of the 3′-trailer sequence chosen indicates that it has a suboptimal processing potential⁴⁰. In order to optimize the 3′-trailer sequence a library of all possible four nucleotide long 3′-trailer sequences cloned between the tRNA and the polyT transcriptional terminator are produced. This 256-member library does not only probe the sequence specificity for 3′ end processing of the ACE-tRNA^{Arg}_{UGA} and ACE-tRNA^{Leu}_{UGA} hits but also can determine optimal lengths of 3′ trailer, as the library contains the four nucleotide sequences NNNT, NNTT, and NTTT.

Each of these sequences shortens the trailer by on average one nucleotide by effectively starting the polyT terminator one nucleotide earlier. The library is constructed and tested as above with a few notable differences. The restriction endonuclease BbsI is used due to its four bp overhang and slightly higher efficiency, the HTC plasmid is altered so that the ccdB negative selection cassette is between the tRNA and the polyT transcriptional terminator, and the insert in this case is annealed oligos as in Lueck et al., Nat Commun 2019, 10 (1), 822. As above, the most efficient 3′-trailer sequence library member can be determined as the one with the highest luminescent ratio over baseline.

### Example 4

This example describes experimental designs and methods for determining the optimal sequences for efficient aminoacylation and transport of ACE-tRNAs both for expression from DNA cassettes and for delivery as RNA.

Previous efforts with GCE have shown the importance of optimizing translational elements of suppressor tRNAs^{2, 4-5, 27-30, 43}. Optimization of the translational elements of ACE-tRNAs is of importance as they can impact efficiency of ACE-tRNA delivered by any method. The experimental designs and methods disclosed here optimize the anticodon loop of ACE-tRNAs for efficient charging by their cognate aminoacyl-tRNA synthetases and their T-stems for efficient transport by EF-1a.

4.1 Determining Optimal Anticodon Loop Sequences for ACE-tRNA^{Arg}_{UGA} and ACE-tRNA^{Leu}_{UGA} to Improve Aminoacylation Efficiency by their Cognate Aminoacyl-tRNA Synthetases

As described above in Example 3.2, a library of all possible non-anticodon nucleotides of the anticodon loop of the ACE-tRNA^{Arg}_{UGA} and ACE-tRNA^{Leu}_{UGA} cloned in the middle of each of these ACE-tRNAs are produced. This is accomplished by inserting a BbsI flanked ccdB negative selection cassette between nucleotides 31 and 39 of each ACE-tRNA hit. The 256-member anticodon loop library is ordered as complimentary oligos (IDT, USA) and cloned into the anticodon loop HTC plasmid. As above, the most efficient anticodon loop library member is determined as the one with the highest luminescent ratio over baseline.

4.2 Determining Optimal T-Stem Sequences for ACE-tRNA^{Arg}_{UGA} and ACE-tRNA^{Leu}_{UGA} to Improve Transport to the Ribosome by EF-1a

Optimization of the T-stem of nonsense suppressor tRNAs for GCE has improved nonsense suppression efficiency 5-25 fold. To optimize the T-stem the three highest efficiency T-stem sequences from the ACE-tRNA^{Trp}_{UGA} screened (FIG. 3) are used with ACE-tRNA^{Arg}_{UGA} and ACE-tRNA^{Leu}_{UGA} hits. As above, the most efficient T-stem library member is determined as the one with the highest luminescent ratio over baseline.

Altering the tRNA body (anticodon loop and T-stem) has the potential to affect the translational fidelity of the ACE-tRNA hits. To validate the translational fidelity of ACE-tRNA^{Arg}_{UGA} and ACE-tRNA^{Leu}_{UGA} containing optimized T-stems and anticodon loops they are co-transfected with pcDNA3.1 histidinol dehydrogenase with a C-terminal 8xHis-Strep-tag for protein purification. The purified protein is subjected to mass spectrometry to show homogeneous site-specific PTC suppression as previously reported¹. If translational fidelity of any of the most efficient library members is compromised, the next most efficient member is used instead.

With ACE-tRNA^{Arg}_{UGA} and ACE-tRNA^{Leu}_{UGA} leads in hand DNA expression cassettes in 16HBE14o− for PTC reporter suppression, CFTR mRNA expression and channel function are assessed as outlined above in Example 1. The best anticodon loop and T-stem sequences are combined in ACE-tRNA^{Arg}_{UGA} and ACE-tRNA^{Leu}_{UGA} lead RNA constructs and delivered to 16HBE14o− cells and PTC reporter suppression, CFTR mRNA expression and channel function as outlined above in Example 1.

### Example 5

The experimental designs and methods described above were used to screen and identify 5′ leaders, 3′ trailers, t-stem mutants, and sticky stem mutants resulting in enhanced RNA expression or nonsense mutation suppression.

First, assays were carried out to identify 5′ leaders that resulted in higher PTC suppression than that of a control or original 5′ leader. Briefly, 55 bp upstream of each human tRNA were ordered as pairs of single stranded DNA oligos (386 members), annealed and cloned via a high throughput Golden Gate cloning strategy using SapI restriction enzyme into the 5′ upstream control element (5′ UCE) screening plasmid (FIG. 2A).

The related library for the 55 bp 5′ upstream leader were assembled as shown in FIGS. 7A, 7B, and 7C. The parent high-throughput-cloning and -screening vector for the tRNA 5′ region, contains a cloning site immediately 5′ to the original ArgTGA tRNA sequence/the original 3′ trailer sequence (FIG. 7A). All human tRNAs are denoted with a name such as like “tRNA-Ala-AGC-1-1” (where Ala is the three-letter amino acid code for the tRNA isotype, AGC is the anticodon, the first 1 corresponds to the numeric ID of a unique tRNA transcript or “isodecoder”, and the second 1 corresponds to the gene locus ID-for tRNAs that have multiple identical copies, this gene locus ID represents the particular gene copy in the genome). Scanning the entire human genome returned 386 predicted tRNAs with a unique 55 bp sequence immediately upstream of the tRNA (FIG. 7B).

To determine the impact of these different sequences on the function of ACE-tRNAs, each unique 55 bp 5′ upstream control element (UCE, what was also called 5′ upstream transcriptional element) was synthesized as complementary oligonucleotides, which when annealed provide overhangs for golden gate cloning into parent vector shown in (FIGS. 7A and 7C). The names in Tables 1A and 1B correspond to the original tRNA from which the 5′ sequence was derived.

The Golden Gate reactions were transformed into chemically competent NEB 5-alpha cells, and miniprepped with Macherey-Nagel Nucleospin 96 Transfection-grade kit. The DNA concentration was normalized to 50 ng/μL, mixed 1:1 (v/v) with 150 ng/μL carrier plasmid (pUC 57 mini), and transfected into 16HBE14o− or HEK293T cells using lipofectamine 2000 reagent. After maintaining cells in a CO₂ incubator for 24 hours, PTC rescue was determined using the Nano-Glo Dual-Luciferase Reporter Assay System, with firefly luciferase (Fluc) serving as a transfection control and PTC-containing nanoluciferase (Nluc) serving as a PTC readthrough control. Average normalized suppression ratio was obtained according to the following:

Average normalized suppression ratio=(PTC-NanoLuciferase luminescence [+ACE-tRNA]/Firefly luminescence)/(PTC−Nanoluciferase luminescence [no ACE-tRNA]/Firefly luminescence).

The results of those exhibiting ratios higher than that of the original one are shown in Tables 1A and 1B.

TABLE 1A
Results of 55 bp leader screen for ArgTGA in HEK293T cells

| | | | Standard | |
|---|---|---|---|---|
| | | Average | Error | |
| | | Normalized | of The | |
| | | Suppression | Mean | SEQ ID |
| Name | Sequence (tRNA gene/expression cassette) | Ratio | (S.E.M.) | NO: |
| tRNA-Tyr- | GATGAGGTTGCGGTAAGCATTAGAGGGCTATCAGCAGCATCTTA | 1949.28 | 67.08 | 1 |
| GTA-2-1 | TCGCAGCGGAGGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Leu- | ATAGCGTTTAGACAAGAGACAGAAGTGAGTCCTGAGCACAATGT | 1890.89 | 119.87 | 2 |
| CAG-1-7 | AGCATAAGCGCGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Pro- | GGTGAGAGGCGGAACAAGGCTGAAGGGAAGCTCTGGGAGTGCC | 1887.73 | 73.48 | 3 |
| CGG-1-1 | CTTGCCGAGAGCGGCTCTGTGGCGCAATGGATAGCGCATTGGAC | | | |
| | TTCAAATTCAAAGGTTGTGGGTTCGAGTCCCACCAGAGTCGGTC | | | |
| | CTTTTTTT | | | |
| tRNA-Glu- | GCGTATGTGTTGAGTTCTCGCTTCATAAATATGTTTTAATAAACC | 1870.04 | 134.20 | 4 |
| CTC-1-6 | TACTTCAGCTGGCTCTGTGGCGCAATGGATAGCGCATTGGACTTC | | | |
| | AAATTCAAAGGTTGTGGGTTCGAGTCCCACCAGAGTCGGTCCTTT | | | |
| | TTTT | | | |
| tRNA-Pro- | ACGCTTGCCGCTGTCATCGCCTCTAGTAAATTCGAGGAGACCTTG | 1833.97 | 37.01 | 5 |
| TGG-2-1 | CTCAGCAGATGGCTCTGTGGCGCAATGGATAGCGCATTGGACTT | | | |
| | CAAATTCAAAGGTTGTGGGTTCGAGTCCCACCAGAGTCGGTCCTT | | | |
| | TTTTT | | | |
| tRNA-Asn- | AGTTTCGTGCGTGCGCCCTTCCTATAGCGCCCAGTAGAACTGACT | 1833.33 | 67.43 | 6 |
| GTT-7-1 | GACAGTACCTGGCTCTGTGGCGCAATGGATAGCGCATTGGACTTC | | | |
| | AAATTCAAAGGTTGTGGGTTCGAGTCCCACCAGAGTCGGTCCTT | | | |
| | TTTTT | | | |
| tRNA-Ile- | GCGGGCGCGGGCTTGGAGCCCTCTTAGGCGCTCCCGTGGGGGCC | 1793.38 | 148.88 | 7 |
| AAT-5-4 | CAACGTCAGGCGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Lys- | AACCATGTAGTTAAACAGTCCTCAAGAAGTAAAAAAGCAGGCTA | 1780.62 | 52.96 | 8 |
| CTT-1-1 | ACGTAACAGGCGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Leu- | ATCATTGCTTGTTGATCTCTATTTTAGTGCAAAAATCAGGGCTGT | 1777.42 | 26.56 | 9 |
| CAA-1-1 | TATGCAGTGTGGCTCTGTGGCGCAATGGATAGCGCATTGGACTTC | | | |
| | AAATTCAAAGGTTGTGGGTTCGAGTCCCACCAGAGTCGGTCCTTT | | | |
| | TTTT | | | |
| tRNA-Asp- | CGGGGCGGAGCAAAAACGTAGAAAAGGCCTATCAGAAGGCTTTC | 1766.73 | 71.57 | 10 |
| GTC-2-8 | TTTCGTGCGGTGGCTCTGTGGCGCAATGGATAGCGCATTGGACTT | | | |
| | CAAATTCAAAGGTTGTGGGTTCGAGTCCCACCAGAGTCGGTCCTT | | | |
| | TTTTT | | | |
| tRNA-Ala- | TGTCCTGAGTTAAGCGACCAATAGGGACCATCTGGGAGAGCTGC | 1751.62 | 57.45 | 11 |
| TGC-3-1 | TGAGAGTAGGTGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Cys- | GCCAGGGGTGTGGCCATACAGGTTTATAGTGGTTAGTAGAGACA | 1715.17 | 97.91 | 12 |
| GCA-12-1 | AGTAAGTGGGTGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Gln- | TGGGGGGGGGTTCTCTGAGGATTAGACAGCCGAAGGCAGCTAC | 1659.78 | 97.68 | 13 |
| CTG-1-4 | ACATGCGTGGCGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Arg- | AAACCCTGCAGTATAACCTTGAAGTACCATCACGGAGAGAAACA | 1638.91 | 243.20 | 14 |
| ACG-1-3 | GTGCCATCTCAGGCTCTGTGGCGCAATGGATAGCGCATTGGACTT | | | |
| | CAAATTCAAAGGTTGTGGGTTCGAGTCCCACCAGAGTCGGTCCTT | | | |
| | TTTTT | | | |
| tRNA-Leu- | GGGGCGCCTCCGTCATCACACTAATATGCCGGCATAGGAAGCCCT | 1626.16 | 68.00 | 15 |
| TAG-3-1 | ACGATCGGGAGGCTCTGTGGCGCAATGGATAGCGCATTGGACTT | | | |
| | CAAATTCAAAGGTTGTGGGTTCGAGTCCCACCAGAGTCGGTCCTT | | | |
| | TTTTT | | | |
| tRNA-Leu- | ACGCATTCATTACAATCGAAGATTATATTAATTTGCGATGGTAAG | 1587.85 | 150.65 | 16 |
| CAA-1-2 | TGTCCCAGGTGGCTCTGTGGCGCAATGGATAGCGCATTGGACTTC | | | |
| | AAATTCAAAGGTTGTGGGTTCGAGTCCCACCAGAGTCGGTCCTTT | | | |
| | TTTT | | | |
| tRNA-Gly- | GAGGACTTCCCGCGTCACAGTCTCTTGTCTCCGCGGGCAGCAACT | 1571.90 | 111.77 | 17 |
| CCC-2-2 | GTCTTAGACAGGCTCTGTGGCGCAATGGATAGCGCATTGGACTTC | | | |
| | AAATTCAAAGGTTGTGGGTTCGAGTCCCACCAGAGTCGGTCCTTT | | | |
| | TTTT | | | |
| tRNA-Ile- | TCAATCTGTAAGAGCATCTTCTCTTATACCTTTCCAAATCCTTATT | 1569.03 | 81.58 | 18 |
| TAT-3-1 | ATTTCTGTGGGCTCTGTGGCGCAATGGATAGCGCATTGGACTTCAA | | | |
| | ATTCAAAGGTTGTGGGTTCGAGTCCCACCAGAGTCGGTCCTTTTT | | | |
| | TT | | | |
| tRNA-Lys- | GCGCCTGTTTTGAGGATTCTGAGTATTAGGGTGGGAAGCAAGTA | 1556.40 | 93.15 | 19 |
| CTT-8-1 | TTATTACATCAGGCTCTGTGGCGCAATGGATAGCGCATTGGACTT | | | |
| | CAAATTCAAAGGTTGTGGGTTCGAGTCCCACCAGAGTCGGTCCTT | | | |
| | TTTTT | | | |
| tRNA-Lys- | CCAAAGAAATTATGTATTCTCTTAGTGTATTGGGGGAAGAGCTAC | 1544.04 | 62.33 | 20 |
| CTT-2-4 | GTTCGGGTGAGGCTCTGTGGCGCAATGGATAGCGCATTGGACTTC | | | |
| | AAATTCAAAGGTTGTGGGTTCGAGTCCCACCAGAGTCGGTCCTT | | | |
| | TTTTT | | | |
| tRNA-Lys- | ATCAAAACAATCTTATTCCTTTGAAAAGATCTAGGAACAACACTTT | 1543.38 | 102.47 | 21 |
| CTT-1-2 | TCTCTAACTGGCTCTGTGGCGCAATGGATAGCGCATTGGACTTCA | | | |
| | AATTCAAAGGTTGTGGGTTCGAGTCCCACCAGAGTCGGTCCTTTT | | | |
| | TTT | | | |
| tRNA-Gly- | ATTACCAGGGTGTAAGTGTCTTCTAGTAAGCAATAGAAAAGCACA | 1543.07 | 78.48 | 22 |
| GCC-2-2 | TTTGCTATAAGGCTCTGTGGCGCAATGGATAGCGCATTGGACTTC | | | |
| | AAATTCAAAGGTTGTGGGTTCGAGTCCCACCAGAGTCGGTCCTTT | | | |
| | TTTT | | | |
| tRNA-Ile- | TCCGAAACACTGATGAAATTCTTGATGCAGTTTTTAAAACTTCTT | 1538.29 | 110.58 | 23 |
| AAT-5-1 | GGAGACAGGTGGCTCTGTGGCGCAATGGATAGCGCATTGGACTTC | | | |
| | AAATTCAAAGGTTGTGGGTTCGAGTCCCACCAGAGTCGGTCCTTT | | | |
| | TTTT | | | |
| tRNA-Tyr- | ATGTGGCTCCCTCTGTTTGCAGAAAGTCCAATGAACCAGCTTTGA | 1529.72 | 180.67 | 24 |
| GTA-4-1 | TAGCATGCATGGCTCTGTGGCGCAATGGATAGCGCATTGGACTTC | | | |
| | AAATTCAAAGGTTGTGGGTTCGAGTCCCACCAGAGTCGGTCCTTT | | | |
| | TTTT | | | |
| tRNA-Leu- | GTCAGCGTCTGCTGACTACGAGTTACATAATGAAACTTAAGGAGT | 1521.72 | 183.23 | 25 |
| CAA-3-1 | AACAGTGCGTGGCTCTGTGGCGCAATGGATAGCGCATTGGACTT | | | |
| | CAAATTCAAAGGTTGTGGGTTCGAGTCCCACCAGAGTCGGTCCTT | | | |
| | TTTTT | | | |
| tRNA- | ATACTTGACAAGAGTAATGTTGAAACAGCAGAGTGGCGGAGCGG | 1511.14 | 77.61 | 26 |
| iMet-CAT- | ACTGTCGAGACGGCTCTGTGGCGCAATGGATAGCGCATTGGACT | | | |
| 1-3 | TCAAATTCAAAGGTTGTGGGTTCGAGTCCCACCAGAGTCGGTCCT | | | |
| | TTTTTT | | | |
| tRNA-Val- | CACTGTGCCCAGCCTCATGGCCTCATAGTCTAGCTTTGAAACTCAG | 1499.19 | 31.63 | 27 |
| TAC-3-1 | CTAGCCTGTGGCTCTGTGGCGCAATGGATAGCGCATTGGACTTCA | | | |
| | AATTCAAAGGTTGTGGGTTCGAGTCCCACCAGAGTCGGTCCTTTT | | | |
| | TTT | | | |
| tRNA-Leu- | TAAATGCAAAAGCAAAAGCATTAAGTAGCTTTCTGGCATAAACAT | 1497.72 | 137.95 | 28 |
| AAG-2-4 | ATTGCAGCTGGGCTCTGTGGCGCAATGGATAGCGCATTGGACTT | | | |
| | CAAATTCAAAGGTTGTGGGTTCGAGTCCCACCAGAGTCGGTCCTT | | | |
| | TTTTT | | | |
| tRNA-Glu- | TACGTCACAGGATATTTATTTAGTATAGCATATTGCTAAATGACGC | 1478.09 | 65.12 | 29 |
| CTC-2-1 | ATCATGTCTGGCTCTGTGGCGCAATGGATAGCGCATTGGACTTCA | | | |
| | AATTCAAAGGTTGTGGGTTCGAGTCCCACCAGAGTCGGTCCTTTT | | | |
| | TTT | | | |
| tRNA-Leu- | CGGGGAGGGTAAAAGAGGAAAGGGTAGAGAATAGGTTCCCAAC | 1476.59 | 64.52 | 30 |
| AAG-2-3 | TGTTAGCCAGTAGGCTCTGTGGCGCAATGGATAGCGCATTGGAC | | | |
| | TTCAAATTCAAAGGTTGTGGGTTCGAGTCCCACCAGAGTCGGTCC | | | |
| | TTTTTTT | | | |
| tRNA-Gly- | CAAACCCATGATCCCCCACTATTAAGGATATCCGGAGAGGATGCT | 1473.85 | 89.54 | 31 |
| TCC-1-1 | ACCTATCAGGGGCTCTGTGGCGCAATGGATAGCGCATTGGACTT | | | |
| | CAAATTCAAAGGTTGTGGGTTCGAGTCCCACCAGAGTCGGTCCTT | | | |
| | TTTTT | | | |
| tRNA-Gly- | ACCTTCCAGTCGCGTTCCAATCATGTTACTGCTTTTCCCCAACTCT | 1468.19 | 79.89 | 32 |
| GCC-1-5 | TCTATCGGTGGCTCTGTGGCGCAATGGATAGCGCATTGGACTTCAA | | | |
| | ATTCAAAGGTTGTGGGTTCGAGTCCCACCAGAGTCGGTCCTTTTT | | | |
| | TT | | | |
| tRNA-Thr- | ACGGAAGTGTGAAATAGTTTTCAAGTAGTATACGATCGAGGGCT | 1467.92 | 184.75 | 33 |
| AGT-2-2 | TTCTCGGCTGTGGCTCTGTGGCGCAATGGATAGCGCATTGGACTT | | | |
| | CAAATTCAAAGGTTGTGGGTTCGAGTCCCACCAGAGTCGGTCCTT | | | |
| | TTTTT | | | |
| tRNA-Glu- | CAAGGGCAGGCGGAAATGATTCAATTGGTTTAGAATTCAACGGT | 1451.18 | 81.73 | 34 |
| TTC-2-2 | TACTTGTCATCGGCTCTGTGGCGCAATGGATAGCGCATTGGACTT | | | |
| | CAAATTCAAAGGTTGTGGGTTCGAGTCCCACCAGAGTCGGTCCTT | | | |
| | TTTTT | | | |
| tRNA-Cys- | CGGCTTGCGTGCGGCTGACTACGAAGACGTGAGTGACGCTTTCCT | 1440.92 | 68.71 | 35 |
| GCA-11-1 | CCCGTGTGACGGCTCTGTGGCGCAATGGATAGCGCATTGGACTT | | | |
| | CAAATTCAAAGGTTGTGGGTTCGAGTCCCACCAGAGTCGGTCCTT | | | |
| | TTTTT | | | |
| tRNA- | AACGCATCTCCTGTAAGTAGTTAAGAGTACTGTGAGACCGTGTGC | 1414.86 | 39.14 | 36 |
| iMet-CAT- | CTGGCAGAACGGCTCTGTGGCGCAATGGATAGCGCATTGGACTT | | | |
| 1-2 | CAAATTCAAAGGTTGTGGGTTCGAGTCCCACCAGAGTCGGTCCTT | | | |
| | TTTTT | | | |
| tRNA-Ile- | AGAACTGAACAGTATTGTAACTAAATAGATTTATTTCAAGCTTTT | 1399.56 | 179.29 | 37 |
| AAT-5-5 | CGTAGCAACTGGCTCTGTGGCGCAATGGATAGCGCATTGGACTTC | | | |
| | AAATTCAAAGGTTGTGGGTTCGAGTCCCACCAGAGTCGGTCCTTT | | | |
| | TTTT | | | |
| tRNA-Ser- | AATGGCACACTGCTCACATCTTCAAAAGCTTAAACCAAGAGCACT | 1396.59 | 179.21 | 38 |
| TGA-4-1 | ACACAGGTGCGGCTCTGTGGCGCAATGGATAGCGCATTGGACTT | | | |
| | CAAATTCAAAGGTTGTGGGTTCGAGTCCCACCAGAGTCGGTCCTT | | | |
| | TTTTT | | | |
| tRNA-Ser- | TACAATATGTGATGTATGGAAACATGTAAGACATTTAATAAGGTT | 1394.01 | 172.73 | 39 |
| AGA-2-1 | TTTGGTATCTGGCTCTGTGGCGCAATGGATAGCGCATTGGACTTC | | | |
| | AAATTCAAAGGTTGTGGGTTCGAGTCCCACCAGAGTCGGTCCTTT | | | |
| | TTTT | | | |
| tRNA-Tyr- | AGCGCTCCGGTTTTTCTGTGCTGAACCTCAGGGGACGCCGACACA | 1047.01 | 90.93 | 40 |
| GTA-5-1 | CGTACACGTCGGCTCTGTGGCGCAATGGATAGCGCATTGGACTTC | | | |
| | AAATTCAAAGGTTGTGGGTTCGAGTCCCACCAGAGTCGGTCCTTT | | | |
| | TTTT | | | |

Note:
1. 5′ leader sequence-oArgTGA-o3′ trailer sequence (o prefix denotes original).
2. The last highlighted row contains the original 55 bp 5′ leader sequence.

TABLE 1B
Results of 55 bp leader screen for ArgTGA in 16HBE140-cells

| | | Average | | |
|---|---|---|---|---|
| | | Normalized | | |
| | | Suppression | | SEQ ID |
| Name | Sequence | Ratio | S.E.M. | NO: |
| tRNA-Cys- | GCCAGGGGTGTGGCCATACAGGTTTATAGTGGTTAGTAGAGAC | 437.39 | 20.57 | 41 |
| GCA-12-1 | AAGTAAGTGGGTGGCTCTGTGGCGCAATGGATAGCGCATTGGA | | | |
| | CTTCAAATTCAAAGGTTGTGGGTTCGAGTCCCACCAGAGTCGG | | | |
| | TCCTTTTTTT | | | |
| tRNA-Ile- | TCAATCTGTAAGAGCATCTTCTCTTATACCTTTCCAAATCCTTAT | 417.08 | 38.56 | 42 |
| TAT-3-1 | TATTTCTGTGGGCTCTGTGGCGCAATGGATAGCGCATTGGACTTC | | | |
| | AAATTCAAAGGTTGTGGGTTCGAGTCCCACCAGAGTCGGTCCTT | | | |
| | TTTTT | | | |
| tRNA-Tyr- | GATGAGGTTGCGGTAAGCATTAGAGGGCTATCAGCAGCATCTT | 405.65 | 19.32 | 43 |
| GTA-2-1 | ATCGCAGCGGAGGGCTCTGTGGCGCAATGGATAGCGCATTGGA | | | |
| | CTTCAAATTCAAAGGTTGTGGGTTCGAGTCCCACCAGAGTCGG | | | |
| | TCCTTTTTTT | | | |
| tRNA-Ala- | TGTCCTGAGTTAAGCGACCAATAGGGACCATCTGGGAGAGCTG | 404.67 | 8.62 | 44 |
| TGC-3-1 | CTGAGAGTAGGTGGCTCTGTGGCGCAATGGATAGCGCATTGGA | | | |
| | CTTCAAATTCAAAGGTTGTGGGTTCGAGTCCCACCAGAGTCGG | | | |
| | TCCTTTTTTT | | | |
| tRNA-Leu- | ATAGCGTTTAGACAAGAGACAGAAGTGAGTCCTGAGCACAATG | 389.16 | 16.45 | 45 |
| CAG-1-7 | TAGCATAAGCGCGGCTCTGTGGCGCAATGGATAGCGCATTGGA | | | |
| | CTTCAAATTCAAAGGTTGTGGGTTCGAGTCCCACCAGAGTCGG | | | |
| | TCCTTTTTTT | | | |
| tRNA-Leu- | TAAATGCAAAAGCAAAAGCATTAAGTAGCTTTCTGGCATAAACA | 386.41 | 47.54 | 46 |
| AAG-2-4 | TATTGCAGCTGGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Pro- | ACGCTTGCCGCTGTCATCGCCTCTAGTAAATTCGAGGAGACCTT | 383.05 | 12.72 | 47 |
| TGG-2-1 | GCTCAGCAGATGGCTCTGTGGCGCAATGGATAGCGCATTGGAC | | | |
| | TTCAAATTCAAAGGTTGTGGGTTCGAGTCCCACCAGAGTCGGTC | | | |
| | CTTTTTTT | | | |
| tRNA-Ser- | AAAGAATAGGAATTGCCAAATGAAACTGTTGAGAAGAGAAACA | 382.54 | 50.71 | 48 |
| GCT-6-1 | TTACACATATCAGGCTCTGTGGCGCAATGGATAGCGCATTGGAC | | | |
| | TTCAAATTCAAAGGTTGTGGGTTCGAGTCCCACCAGAGTCGGTC | | | |
| | CTTTTTTT | | | |
| tRNA-Ser- | AATGGCACACTGCTCACATCTTCAAAAGCTTAAACCAAGAGCAC | 378.65 | 39.12 | 49 |
| TGA-4-1 | TACACAGGTGCGGCTCTGTGGCGCAATGGATAGCGCATTGGAC | | | |
| | TTCAAATTCAAAGGTTGTGGGTTCGAGTCCCACCAGAGTCGGTC | | | |
| | CTTTTTTT | | | |
| tRNA-Leu- | CTGGCACCAGCGGACACAAGTATGAGGCTAAGTGAGAAGCGCT | 376.75 | 26.67 | 50 |
| CAA-4-1 | CTGCTTCCGAGTGGCTCTGTGGCGCAATGGATAGCGCATTGGAC | | | |
| | TTCAAATTCAAAGGTTGTGGGTTCGAGTCCCACCAGAGTCGGTC | | | |
| | CTTTTTTT | | | |
| tRNA-Leu- | AGGAGGAGGTAGAGGATGCCTTGTAAGCGGAGCAAAAACAAG | 376.24 | 52.73 | 51 |
| TAA-1-1 | GTTCAACGTCTGCGGCTCTGTGGCGCAATGGATAGCGCATTGG | | | |
| | ACTTCAAATTCAAAGGTTGTGGGTTCGAGTCCCACCAGAGTCGG | | | |
| | TCCTTTTTTT | | | |
| tRNA-Leu- | ACGCATTCATTACAATCGAAGATTATATTAATTTGCGATGGTAA | 376.16 | 30.69 | 52 |
| CAA-1-2 | GTGTCCCAGGTGGCTCTGTGGCGCAATGGATAGCGCATTGGAC | | | |
| | TTCAAATTCAAAGGTTGTGGGTTCGAGTCCCACCAGAGTCGGTC | | | |
| | CTTTTTTT | | | |
| tRNA-Pro- | GGTGAGAGGCGGAACAAGGCTGAAGGGAAGCTCTGGGAGTGC | 375.15 | 19.23 | 53 |
| CGG-1-1 | CCTTGCCGAGAGCGGCTCTGTGGCGCAATGGATAGCGCATTGG | | | |
| | ACTTCAAATTCAAAGGTTGTGGGTTCGAGTCCCACCAGAGTCGG | | | |
| | TCCTTTTTTT | | | |
| tRNA-Leu- | GGGGCGCCTCCGTCATCACACTAATATGCCGGCATAGGAAGCCC | 375.09 | 15.01 | 54 |
| TAG-3-1 | TACGATCGGGAGGCTCTGTGGCGCAATGGATAGCGCATTGGAC | | | |
| | TTCAAATTCAAAGGTTGTGGGTTCGAGTCCCACCAGAGTCGGTC | | | |
| | CTTTTTTT | | | |
| tRNA-Lys- | AACCATGTAGTTAAACAGTCCTCAAGAAGTAAAAAAGCAGGCTA | 373.33 | 3.97 | 55 |
| CTT-1-1 | ACGTAACAGGCGGCTCTGTGGCGCAATGGATAGCGCATTGGAC | | | |
| | TTCAAATTCAAAGGTTGTGGGTTCGAGTCCCACCAGAGTCGGTC | | | |
| | CTTTTTTT | | | |
| tRNA-Trp- | AGAACCATACTGCAGAAACGAGGTCTTTAGCGATGCGGAGTCA | 371.17 | 39.59 | 56 |
| CCA-2-1 | CCACTTTGGTGTGGCTCTGTGGCGCAATGGATAGCGCATTGGAC | | | |
| | TTCAAATTCAAAGGTTGTGGGTTCGAGTCCCACCAGAGTCGGTC | | | |
| | CTTTTTTT | | | |
| tRNA-Thr- | ACGGAAGTGTGAAATAGTTTTCAAGTAGTATACGATCGAGGGC | 370.44 | 42.63 | 57 |
| AGT-2-2 | TTTCTCGGCTGTGGCTCTGTGGCGCAATGGATAGCGCATTGGAC | | | |
| | TTCAAATTCAAAGGTTGTGGGTTCGAGTCCCACCAGAGTCGGTC | | | |
| | CTTTTTTT | | | |
| tRNA-Ile- | GCGGGCGCGGGCTTGGAGCCCTCTTAGGCGCTCCCGTGGGGGC | 367.51 | 13.24 | 58 |
| AAT-5-4 | CCAACGTCAGGCGGCTCTGTGGCGCAATGGATAGCGCATTGGA | | | |
| | CTTCAAATTCAAAGGTTGTGGGTTCGAGTCCCACCAGAGTCGGT | | | |
| | CCTTTTTTT | | | |
| tRNA-Gly- | GAGGACTTCCCGCGTCACAGTCTCTTGTCTCCGCGGGCAGCAAC | 367.48 | 20.99 | 59 |
| CCC-2-2 | TGTCTTAGACAGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Leu- | GTCAGCGTCTGCTGACTACGAGTTACATAATGAAACTTAAGGAG | 366.79 | 20.12 | 60 |
| CAA-3-1 | TAACAGTGCGTGGCTCTGTGGCGCAATGGATAGCGCATTGGAC | | | |
| | TTCAAATTCAAAGGTTGTGGGTTCGAGTCCCACCAGAGTCGGTC | | | |
| | CTTTTTTT | | | |
| tRNA-Cys- | CTTGATTGTATTGAGTCACCATAAGTGGACAAGGAAGGAACCTC | 362.59 | 53.35 | 61 |
| GCA-5-1 | TAACAGTTGAAGGCTCTGTGGCGCAATGGATAGCGCATTGGAC | | | |
| | TTCAAATTCAAAGGTTGTGGGTTCGAGTCCCACCAGAGTCGGTC | | | |
| | CTTTTTTT | | | |
| tRNA-Asp- | CGGGGCGGAGCAAAAACGTAGAAAAGGCCTATCAGAAGGCTTT | 359.04 | 10.04 | 62 |
| GTC-2-8 | CTTTCGTGCGGTGGCTCTGTGGCGCAATGGATAGCGCATTGGAC | | | |
| | TTCAAATTCAAAGGTTGTGGGTTCGAGTCCCACCAGAGTCGGTC | | | |
| | CTTTTTTT | | | |
| tRNA-Leu- | ATCATTGCTTGTTGATCTCTATTTTAGTGCAAAAATCAGGGCTGT | 358.25 | 14.76 | 63 |
| CAA-1-1 | TATGCAGTGTGGCTCTGTGGCGCAATGGATAGCGCATTGGACTT | | | |
| | CAAATTCAAAGGTTGTGGGTTCGAGTCCCACCAGAGTCGGTCCT | | | |
| | TTTTTT | | | |
| tRNA-Gln- | TGGGGGCGGGGTTCTCTGAGGATTAGACAGCCGAAGGCAGCTA | 357.39 | 41.02 | 64 |
| CTG-1-4 | CACATGCGTGGCGGCTCTGTGGCGCAATGGATAGCGCATTGGA | | | |
| | CTTCAAATTCAAAGGTTGTGGGTTCGAGTCCCACCAGAGTCGGT | | | |
| | CCTTTTTTT | | | |
| tRNA-Arg- | TGCCCCATTCCCACTCTAGTACCCGTAAGCTACAAGACGCCGCCG | 351.24 | 42.10 | 65 |
| TCT-2-1 | TTCGTCGGGTGGCTCTGTGGCGCAATGGATAGCGCATTGGACTT | | | |
| | CAAATTCAAAGGTTGTGGGTTCGAGTCCCACCAGAGTCGGTCCT | | | |
| | TTTTTT | | | |
| tRNA-Cys- | CCTTGGGTGTGGCAACACAGGTGAAATATGCTAAGGAGGAATG | 350.82 | 29.15 | 66 |
| GCA-23-1 | TCCACCAGGCAAGGCTCTGTGGCGCAATGGATAGCGCATTGGA | | | |
| | CTTCAAATTCAAAGGTTGTGGGTTCGAGTCCCACCAGAGTCGGT | | | |
| | CCTTTTTTT | | | |
| tRNA-Leu- | CGGGGAGGGTAAAAGAGGAAAGGGTAGAGAATAGGTTCCCAA | 349.19 | 22.59 | 67 |
| AAG-2-3 | CTGTTAGCCAGTAGGCTCTGTGGCGCAATGGATAGCGCATTGG | | | |
| | ACTTCAAATTCAAAGGTTGTGGGTTCGAGTCCCACCAGAGTCGG | | | |
| | TCCTTTTTTT | | | |
| tRNA-Lys- | TCACGGCATAATGGTAACAGAGTCTTCATTAATGGAAGCAACGT | 348.24 | 18.72 | 68 |
| TTT-3-4 | TACTACCGTGAGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Asn- | AGTTTCGTGCGTGCGCCCTTCCTATAGCGCCCAGTAGAACTGAC | 346.07 | 43.85 | 69 |
| GTT-7-1 | TGACAGTACCTGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Ser- | TACAATATGTGATGTATGGAAACATGTAAGACATTTAATAAGGT | 345.52 | 30.46 | 70 |
| AGA-2-1 | TTTTGGTATCTGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Ile- | TCCGAAACACTGATGAAATTCTTGATGCAGTTTTTAAAACTTCT | 341.41 | 50.76 | 71 |
| AAT-5-1 | TGGAGACAGGTGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Lys- | ATCAAAACAATCTTATTCCTTTGAAAAGATCTAGGAACAACACTT | 339.81 | 11.38 | 72 |
| CTT-1-2 | TTCTCTAACTGGCTCTGTGGCGCAATGGATAGCGCATTGGACTT | | | |
| | CAAATTCAAAGGTTGTGGGTTCGAGTCCCACCAGAGTCGGTCCT | | | |
| | TTTTTT | | | |
| tRNA-Pro- | ACAGCAGGCGGAAAAGAGAAATTCAGAGTGATAGGAAGCACC | 339.65 | 26.51 | 73 |
| TGG-3-2 | ACTCAGCTACAGTGGCTCTGTGGCGCAATGGATAGCGCATTGG | | | |
| | ACTTCAAATTCAAAGGTTGTGGGTTCGAGTCCCACCAGAGTCGG | | | |
| | TCCTTTTTTT | | | |
| tRNA-Leu- | GAGCCGTAAGCCGTGATTACTCATCATGTATAGTTTCAAGGATT | 339.11 | 31.63 | 74 |
| CAA-2-1 | TTTGTCACAGTGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Ala- | GTAAATTTCTATATTCTTTCATAAAAGCAGTTTAAAGACGAACGT | 338.34 | 48.50 | 75 |
| AGC-4-1 | TTTTCGAGGTGGCTCTGTGGCGCAATGGATAGCGCATTGGACTT | | | |
| | CAAATTCAAAGGTTGTGGGTTCGAGTCCCACCAGAGTCGGTCCT | | | |
| | TTTTTT | | | |
| tRNA-Lys- | TGGCTTTTCCTATAGAGAACCCTCTTATAAGCAAAAAGTAAAGCT | 337.89 | 28.90 | 76 |
| TTT-3-5 | CTCGTGAAGAGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Val- | CAAAATGTCTTTTTGAACAATTATCAAATCCTGGAGGAACGTTT | 335.73 | 44.64 | 77 |
| TAC-1-1 | TAGTCGTGTGGGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Cys- | CGGCTTGCGTGCGGCTGACTACGAAGACGTGAGTGACGCTTTCC | 333.85 | 10.61 | 78 |
| GCA-11-1 | TCCCGTGTGACGGCTCTGTGGCGCAATGGATAGCGCATTGGACT | | | |
| | TCAAATTCAAAGGTTGTGGGTTCGAGTCCCACCAGAGTCGGTCC | | | |
| | TTTTTTT | | | |
| tRNA-Lys- | CAGAGGCAGGGAGATTAGGGGAAAGGGAGAATGGGGAGAAACT | 333.65 | 37.25 | 79 |
| TTT-2-1 | GAGTAATGGGTAGGCTCTGTGGCGCAATGGATAGCGCATTGGA | | | |
| | CTTCAAATTCAAAGGTTGTGGGTTCGAGTCCCACCAGAGTCGG | | | |
| | TCCTTTTTTT | | | |
| tRNA-Tyr- | AGCGCTCCGGTTTTTCTGTGCTGAACCTCAGGGGACGCCGACA | 224.96 | 12.38 | 80 |
| GTA-5-1 | CACGTACACGTCGGCTCTGTGGCGCAATGGATAGCGCATTGGA | | | |
| | CTTCAAATTCAAAGGTTGTGGGTTCGAGTCCCACCAGAGTCGG | | | |
| | TCCTTTTTTT | | | |

Note:
1. 5′ leader sequence-oArgTGA-03′ trailer sequence (o prefix denotes original).
2. The last row contains the original 55 bp 5′ leader sequence.

The above described assays were also carried out to examine 850 bp versions of the 55 bp 5′UCE sequences (extending further to the 5′ of the original tRNA gene). The results of those exhibiting ratios higher than that of the original one are shown in Table 1 C below.

TABLE IC
Results of 850 bp leader screen for ArgTGA in HEK293T cells

| | Average | | | |
|---|---|---|---|---|
| | Normalized | | | |
| 5′ Leader | Suppression | | | SEQ ID |
| Name | Ratio | S.E.M. | Sequence (850 bp 5′ leader) | NO: |
| tRNA-Leu- | 1742.76 | 143.61 | TTTCCAGCCCCCTGAGATCCTAGAAATCCTTCTGGAGAAGGGCACCCTCTCGG | 81 |
| CAG-2-2 | | | TACCGAGGTCTTCTGGTCCCTGGAAACAGCCTGTGCGCGGCGTGCGTAAGCCT | |
| | | | AGAGGGGGTGGAAGGGAAGAGAGGAAGATGCCGCGGGGGCCAGGAAAAG | |
| | | | AGAGATGAGTCACGAAATGAGGGGATGCAGGAAGGAGGAAGCGAGGGAAG | |
| | | | AATGAATCCATGGGATCTTCCCTGCGTACCTACTGTGTGCCGGGTACGGTTTG | |
| | | | GGCACTGGGGACATAGGAATAGGGCGGGTCTGGCTGTGCAGCTGCAGGCCT | |
| | | | CGTTCTATCCCGGAGCCTGAGTAAATGAATTATAACACATATACAAATGTGAA | |
| | | | GCTGCAGCTGGGGCGAGTATTCGGAAGGAGAGCGCGGTAAGAGTGCCAGGC | |
| | | | GGCCTTGCAATACCTGGGCTTTACCTAGATTGGGCGCCAGCCCACGGAAATGT | |
| | | | CTGCGAATCAGTGCAGGTATACGGACTTGATTGCATTGTGCACAAGGGAAAT | |
| | | | GCAAGGTTGCTTGCAGATTTCAGGAATCAAAGCTGACATGATAGTACATGTAT | |
| | | | GAATGCAGAATGAATACAGGCTGGAGGCACGAATGCATGTGTATTGAAGTAA | |
| | | | CAGTCTTCGGTCTCGGCCTTCTCTAGTAGGCCTTGCGCACCCTCTGGCGTCGCC | |
| | | | TAGGTGCATCGCGCTGACCTTCCTTGTCCCATCTCTCCACGCCCATTGGAGCAA | |
| | | | GTGAGTGTATCTCGCTCCACCCAACGCCGGGCGGACAACTAGGAGTGGGTCT | |
| | | | TCGCTGCCCACTACGCATGCGCATATTGCAGAACTTGTGAAGAGGCTTTATGC | |
| | | | GCCACACACTGCAAGCA | |
| tRNA-Pro- | 1602.41 | 105.89 | TTTGAAAGGGCCAACTTAGCTCCTGCCCCCGCAGAGCTTCCAGTCTGAGTCGC | 82 |
| TGG-2-1 | | | TGGAACTGGGAAAGAACAGAGGACTCCAGGAGGTTGGGGTTGACCCTGAGC | |
| | | | GGAGAGGAAGGACGAAAGGTAATCGGTAGGTGAGTGTTTGGGGGTAATGTT | |
| | | | CGTTAATGCAAAAAATAAGAAAAACCCGCACAGCCAGGCACAAGTGCTTGCC | |
| | | | CTCCGCGCCAGACCCTCGAGCGCACTCGCGCTCCACTTTTGCATCCACTGCTCC | |
| | | | AGGCCCGGTGCTCCCTCCGCCGCGCGGCCAAGAGGTCTCCATTTTGGGGTAGC | |
| | | | TGCGGAAGGCTCGCTTCTTAGTCGCTGCCCCGCCCCTGTAGCGTTGAGGCGTC | |
| | | | CCTCGCCTCGCTCCCACTAATCCTTCTGTGTAGCCCTCGTCCGCGGCCTGCGCA | |
| | | | CCCTCCCCTCCGGCCCTGGACTGACACATCGCTTTTGGGGTCGATGCACTGTGT | |
| | | | CCAACATTCGGCCTGAATTGATTAAGCACTTGCTGTATGCCGAGCCTGAGGCA | |
| | | | GCCACTGTCCGGGCGCTGGAGGGGGGAAGCATGGATCAGGGATTCCAAGGC | |
| | | | GGGGGCGGGTCCCGCCGGTGGTTGAGGGTTCGCGGGGACAGGAGTTGAGG | |
| | | | GGCAGGGAGGAAAGGGTTTCTGGGTAGCATGGGGGCGCCGTACGTTCTTAG | |
| | | | GAGCAAAGTCGGGAGACATTAGCAGGCCTGAATCCCTGCGTGTCCAGGGAGA | |
| | | | ACGCGTGGATTTCTATGCACAGAAGCCACGCCCCCACCCAACTCTTTCCCGCAA | |
| | | | GCGCTGGCCCACACGCTTGCCGCTGTCATCGCCTCTAGTAAATTCGAGGAGAC | |
| | | | CTTGCTCAGCAG | |
| tRNA-Tyr- | 1302.19 | 172.59 | TTTAAAGACCCACAAGGGAGGCCAGTGGCCTGTGCTGGGCACTGTACAGCCG | 83 |
| GTA-2-1 | | | CCCCGACCCCGACCTCACGTGGGCCTCGGAGGCTGCAGGGACCTACCCGGCC | |
| | | | GCGCGCCGCCCACCATCCCAGGTCTACCGCAGGGGAATCCCCCAGGCTTGCG | |
| | | | AGCGCTTCCGAGGGCAGGAAAGCCTGTACTGCAGCCCCGCCGCCCTCTCCTGG | |
| | | | CGCGTAGCCCTCTCCCCGAGCCCTCGGGCCACGCCGCCAGGCCCGCGATCCCA | |
| | | | GGGCGGCCCGGGGGGTTCGGCACAGCCCGATAATGCGCCGCGCCGAAGCCT | |
| | | | GCAGCACCCCGCGGTCGCCGTCTCTCTTGCACTGGTCAGGACGAATAGAGCCT | |
| | | | GACAAAACCTGAGTTAATTGGCCGAGCGAATGTCGTTCGCGTCAGGAGAGCA | |
| | | | CGTCCGGAGAAGCCAAGGGAAATGTGGAGCAGGAGCAGCCAAAGTGGCCCA | |
| | | | TCGGCTCTCGAGCAGGGTTTGCGCGTCGCGGTCGCAAAGCCCCTGTGAGTGC | |
| | | | AGGTACCGCGGCACAAGCGCTGCCCCGGAGCTCCAGGAGGCGGCGGCGGGC | |
| | | | TCGGCGTTCGCGGTCCAGGAGAAACCACCGCGGGGTGTTGGGAACATTTTGT | |
| | | | TAAAAAGCCGGGAAGCTCAGGGCCGGTGTGGCCTCCGTTCTGGCCTGAGAAC | |
| | | | CCGGACTTTGGGCAGCTTCTTTTCCTTGCCTTTGGGTCTCCGGCTTTCTCAGCTC | |
| | | | TGTGAGGTCCCATGGGTCTCCTGCCCTGGCAGGTTCCTTCAGCTGGAGAAAGC | |
| | | | GGGGGTCATGGCTGGATGAGGTTGCGGTAAGCATTAGAGGGCTATCAGCAG | |
| | | | CATCTTATCGCAGCGG | |
| tRNA-Leu- | 1238.23 | 64.38 | TTTGGTTCTGTGGTAGATATGAGGTCCTGGGTAGTGGGGAATGGAGTGGAAG | 84 |
| TAA-1-1 | | | TCCTGGGAAAAGGGATAACAGGACAAACTGAATCTCATGTGCACAGAAAGGA | |
| | | | AGGACAGCATTGCAAAACAAGGAAGGGGAACAGAAATATCTCATTGCATCGT | |
| | | | AGGGTAGCGGGGGCAAAGAGGAGGCCACTGCGCTGCATCCAAGACTGTGCA | |
| | | | GTGGCGATGGTGCCTTCAGCCACCAGGAAGGGCGACTTTGCCACGCTCCCCTG | |
| | | | CATGTGGCGTGGCACAGCCGACACGCGCATTTCTACAAAGCCTCCCTCCCGTG | |
| | | | TGACTTGTGGCACGTGCGAAGAAGAAAAGCGGCCCCTAGGGGCAGGGGTCC | |
| | | | AGGAACTCTGCTGCTGCCCGTGCACCTCATTCCTTGAAACCTACTCTGCTTAGA | |
| | | | ACAACACAAACTAAACGCCGCCCTGTGGGCCGCGTCCCTGCACCACCAACCAG | |
| | | | GCGCCTCCAGAACGCGCTCCCAGGAACCCAGAAGTTTGCTGGTGTGTGTTTGC | |
| | | | GGCGCCCGCGCACCTCGAAACTGAAAGCGCAACTGGAGGGCCTGGAGTCTCG | |
| | | | CCCCAGCCAAAGGCCCGCGAGCGCCGCTGCCGCGGCCTCCACCCCAACCGAG | |
| | | | AGCGCGCCGCTCCGCTGCAGTTGTCTTCACTGCCGCAGCGGGCCGGGGGCAA | |
| | | | CAGTGAGCCGCGGCGGCGAGAAACCGAGGTACCGGCGTGGCGGCCGCCCCA | |
| | | | GCCCGGGCTGAGAGAGCCGAGACAGTGGCGGCGAAAACTCTCTGCGTTCTGG | |
| | | | AGGGAGGGTGCGGGCAGGAGGAGGTAGAGGATGCCTTGTAAGCGGAGCAA | |
| | | | AAACAAGGTTCAACGTCT | |
| tRNA-Gly- | 1223.04 | 126.96 | TTTTGCCCTGGTCGTTGGCCAGGATCTCCACGCGGCCCTGCTGAAACACGCCC | 85 |
| GCC-2-1 | | | ACGCACGAGTAGGTGGTGCCCAGGTCGATGCCCACCGCGAGCTCCCGTGGGG | |
| | | | CCTGCATGGCTGAAGCTTCTTGTCGGATGCTGGAGGCCACGGAGGCTGCTGC | |
| | | | CGCTCCGCCGTCGAGGGGCGCTCAGCAGTGAGGCTCTCCCTGCGGTTTCTCTG | |
| | | | CAGCCAGCCCGGCTCGGATCTGCTCAGCTCCGCTTCCACCGGCTTTTTATCCTT | |
| | | | TTGCGCCTCACCCGACCCCCGCCGCCGCGAACTTTCGCGAACCTTTCCGCACCT | |
| | | | TCCCGCACCTTCCCGCCCAGTTGAGAGCCTCTCGCCCGCCCGGGCTGACTCAG | |
| | | | CCGGGTGGCGCGGCGCGGCGCTGACCCCGGGAGAAGGTTCGGGCAGATCGG | |
| | | | GGCATGAGCCGCGAGGCTGGGCGGCTACGCCAGGCCAGCCCGCCTCTGACTC | |
| | | | AGGCTGCTGAAAGAAATGCGGGGAAGGTTCTAGCTCCTCCGGGCTGGCTCTG | |
| | | | AGGACGCGCGGTTGTGGCAGAGGCTGCAAAAGCAGCAACTTTAGAGGCAAG | |
| | | | GTCTCCCCGGGGCTGTGGGAGGTCGGATCCCTGAGGCACACAGGGGCCTGCG | |
| | | | GGCTGCCTTGGAGGGCCGCTGAGAGCCCAGCGGCTCAGGAAGGCCGAAGAT | |
| | | | ATGTTACCATGCGAATACAGATATATAAGATATATACACGTATATCTTAAAACA | |
| | | | GTTTTATCTTGAACAAAATATTTTCCAAACCGCGATTTAAGACGGTGCCTGGTT | |
| | | | CCTTAGTGAATGGCCAGAGATAGTGAGCTTTAAGAGACAATACTGCAGGTCTT | |
| | | | TAGCTGACAG | |
| tRNA-Thr- | 1222.47 | 195.17 | TTTGCACAGCGTGCATCAATCCAGAAATAGGCTGAGATAGGAGCTTAGTTTCC | 86 |
| AGT-1-3 | | | TATTGCACACCATGCTGGGTGAACCTTTCTGTGCATGCTGCAGGGATGCTGGG | |
| | | | TGGATGGGTTGGCAGTGTTCGGCTTCGTAGTTGCCCACAGGCCCTCTGGAAA | |
| | | | GTGCGGAGACCAGTGGCTGGTGTAGTTGCGTGAGGACACCCAGAAGGGGTC | |
| | | | AGTGAGAAACTAGGGAGGGGAGGGCCTTGCCTGCTCTGGCAGCAGAAACCC | |
| | | | CTCCATGTAAGGTGTTGGTGCTCCACGCCAGCAGCCTGGGAACCCTGAAGTCC | |
| | | | ACGATCCCTCCTGAGCCTTCCGGACCCCTCGACTTTCCCCCAGCTCAGGATCCG | |
| | | | CGGAAGTAGCGACTCAACTGAAGTTATAGTCCCGGGAGCTGGCAGGGCCCCG | |
| | | | GGTCTTCTCAGGCTGAGGTCGCGGGGCAGAATGGTCCGAAGCTCACCGGGGT | |
| | | | CACAGTCGGACCGAGGCGGGTGTGCGCCTCGACCGGACGCCAGGGGAGCCA | |
| | | | GCAGCCCGGCCGCGCTGGCGGGGCCGGGGGTTCTCCGGAGTGGGTAGGGGC | |
| | | | TCGACCGCACTGCCCTGAGTCCCAAGCCCTGCACCAGCGTTCCCCAAAGGCGG | |
| | | | AGCGTCCTGGCTCCACGTTGGAAGAGACATTACCGTGACCAGGAAGTCTGGC | |
| | | | CTTCCCAGGCTCCGCCCGCTCCGTGCAGCCCAAGCTTCCGGTCTCAGTCTCAGC | |
| | | | TGCCTGTGTGAATCGGAAGCTACACTACGCGTGCGCCACAACAGGCCGAATG | |
| | | | TGTGCGCATGCGCGCGGCCCATATGGTAAATAGTAATGAAGAATAGTTTAGG | |
| | | | AAAGTACCTGTACGG | |
| tRNA-Thr- | 1192.60 | 143.47 | TTTCAAAGGGCATGTTTGAATCTATAAATATGAGCGTGCACATTAAACTGTCA | 87 |
| CGT-2-1 | | | GGTGCCTTGGGGCTGACTTAGCCGACAAGTCAGCAGGTCTGGGCGGGAGAG | |
| | | | AAGTTGCATTTCTCACAAAATCCCAGGTGCTGCTGCTGGTCTTGGGACCAGGG | |
| | | | ACACATCCAGGCTAAACAGGTAAACTGGGGGAAGAAGAAACTTTCGGCTTTG | |
| | | | TTTGTTTGTTTTTATTTTTTTCTTTTCTTTGAGACAAGGTCTCCCTCTGTCGCTCA | |
| | | | GGCTGGTGTGCAGCGGCGCGATCTCGGCTCACTGCAGCCCCGATCTCCTGGG | |
| | | | CTCAAGCGATCCTTCAGCCCGGGCCAAAGCGCGGAGCTTCCAGAAGCTTTTAT | |
| | | | TTTCTAAATCTCACGCCTCCATTCAGCAACCCTGGCTGGGCGTGGGCTCCCTGC | |
| | | | AAGGCGGCGGTGGACGGCCCGCAGTCGGGCCGATAAGGAAACCTGCTACCA | |
| | | | TTCCTTGGCTTCCTGGGTGCCTCACTCTAGAGGGGGAAACTGAGACTCCGAGT | |
| | | | CGAGCCGTCCCGAAGCGCTACATCGCTGGCGGGGCTGGGATTCGAACCCGAC | |
| | | | TTCTGAGATACAGGCCGGTGCCCCGCCCGCGGGACCTGCGGGACCCTCGGGA | |
| | | | CACCGCTCCAGCCGCGCCCCGACTCCAGAGGTTCTCCAGCCCACTGCAGACGA | |
| | | | CAGAATCGCCCGCGGGTCCCCGAGCCCCCGAGCCCGCCGGGGCGCGCCCGTT | |
| | | | GAAGCCCCGCCCCGCCGGCCGCAGCAGGGAGGGGGACGGTGCGAGCTCGTG | |
| | | | CTCGCTGGGCGGGCGCACGGGAGCGGCGCCAGGATAGAGGTTCGCGAGACC | |
| | | | GCCATCGCAACG | |
| tRNA-Leu- | 1191.45 | 177.78 | TTTCCATTTCTTTGGGAGATCCTCAAATACCAGTGTTTGTCGTTGTTTTAATTTT | 88 |
| CAA-1-2 | | | TATTTTTTCCTTGTAAATTGACTTGGATATCTCATCTGTCTTTCTTTATCTCTGGA | |
| | | | AAACTTTATCCCAGTTTCCCTTGACATACTCCTTCAACCTCCTGGGAGAGGTAG | |
| | | | GGGGAGGGGAGATGAGCCTGTTTGACTAGTTCTGGGAATGTGGTGAGGGAA | |
| | | | GAGATCTGGGGATTTCGGCTCAGTGCTGACACTTCCTTCATGCTACTCTGGTTT | |
| | | | AGCACCCCTGCACTTCACCCTCTTTCAACTCCTGACCAAGTCAACTCCTGTTACT | |
| | | | CACATCGACAGTCATTTTTCATGTTCAAGACCCCTGAACCGCTCTCATCCCCCA | |
| | | | CTCTCCGCTAACTCTCCTGCTTCGCTTCAGGTCTACATTGCCTCGCTCATTTCTT | |
| | | | CCCTGCTGAGCTCTTTGATTAGTTTGCTAAGCAGCATCTCGGGAACAGCCCCC | |
| | | | GTGCTATGTGTTCTTTCTTGTCATGGATATGTGTTTTTTCTTGTCATGCTATGTG | |
| | | | TTCTTTCTTGTTCAAGGCAGAGTCGCGGCTGGGTTGTTAGGATGCTCGCGCCC | |
| | | | CTGTACCCGGGGCGTGCAGGGAAGTGTAAGAAGGGGGCGAAGGGCGAGAA | |
| | | | ACGCTGAATTCTAGCCTGAATTGGTAGGAGAGCCTCGCAAGCTGAGTCACGG | |
| | | | TTCCTGATTCCATTTAAATAGAACGTTTTGTTCACTTGTAGGAATTTCCTTTTCC | |
| | | | ATTTCTTTCATTAAATCGCGAACGTAGTAATGACTTAACGCTGGTTACGCATTC | |
| | | | ATTACAATCGAAGATTATATTAATTTGCGATGGTAAGTGTCCCAG | |
| tRNA-Lys-TTT- | 1174.45 | 111.17 | TTTTGCGGAGGGAGGGATCAGATGCTGAGATGGAGAAAAGGAGGTAACGCC | 89 |
| 3-5 | | | TGTATAATTGGGACACTGCCCTCCGCCTGGTCAGCGGCCCGGACTGATGCCCT | |
| | | | GGAGTGCTTGTCCGCCCACTCCCCAATGTCCCCCAAGATCTGTTCCTCCCCATT | |
| | | | CCCAGGCAGAGATGAGCACAGGGCACCTGCATTCCTACGTGTGAATCATCCGT | |
| | | | GTGAATCACTAAACAGTGGAGAGTTGCACTTGGTCATAAATCACACGTGACTG | |
| | | | CAGCCACACGCAGGTGAGTCGCACACGCATTCCAGCACATGTCAAATGGTCA | |
| | | | GGAACAGCTCCCTGTCACCCCATACACCGATCCCCCCACGCATGGCCCTCTTTG | |
| | | | TCCCCAGCCTTTGGCACGCCCGACCTCATTCTCTGCTCTGGAGTAGGGCAGGT | |
| | | | CAGGAAAGCGAAATACAGCGCCGGCAAACAGGGTCTGAATGCCCCGCGTGG | |
| | | | CATTCTTTCCGCTCCAGGACCGCCCTGGGCCTGCAGGATCCTGGGGGGAGCC | |
| | | | CAGGTGTCCGGGATCTGGGCCACTAGGGACTGGGGAGGAACCTCTCAGAGA | |
| | | | AGCCCATAGCCCGCAGCGGCCCCGCGCGGCCGGTTCCGGCGCCGCACTGTTC | |
| | | | CAGCCTCTACTATGGTACAGTCCCTGCGTCGCAGCCTCGGCGGGGGCTCTAAG | |
| | | | AACGGGAGGCAGAAAAAGCTCAATCAGCAGCAGGCGAGCTTCACCCGCTGCT | |
| | | | TCCAAATCTGTGCCAAAATATTCTATGCTGCACAGATAAAATCCTCTGTCGGTT | |
| | | | CTACAAGCCTGGCTTTTCCTATAGAGAACCCTCTTATAAGCAAAAAGTAAAGCT | |
| | | | CTCGTGAAGA | |
| tRNA-Ile-AAT- | 1173.10 | 167.81 | TTTGGTTTTCCACACTTTGAAAACTGATGGATGCTGCCCCATTAACCATTTCAG | 90 |
| 5-1 | | | GATTTTGTTCGACAAATAAAAGAGAATTTAGTTTCAGCAGTCCGCAAATCTTA | |
| | | | GAATTTTGGGGACAGAGATGGGGACGGGGACAGATATGGGAGGACGGAGA | |
| | | | CGGCGGGGCGGTGGGGTGGTAGCAGAAATCTTTCACAAACAACTAGGGAGA | |
| | | | GCTACAAAGAACGGTTTCTTAGGGTAGGGGATGTAGCTCAGTGGTAGAGCGC | |
| | | | ATGCTTCGCATGTATGAGGTCCCGGGTTCGATCCCCGGCATCTCCAAGTTGTTT | |
| | | | TATCCTATCTGCCCAATACTATTAGAAACTATATTTCAAAATGTGAAATAATTG | |
| | | | TGTTTTGTATCCTCCACTTCTTTAACTTTTTATGGCTTTCTTTTAGCCACTGTATG | |
| | | | TAGAATTGTATATGGAGCACGGGGAACAAGATTGCTTTGAAGTATTTTGAACT | |
| | | | ATCTATGTGAAACAAATGGAATTGAGAACACAGAAAAACATGCCCAATGGGG | |
| | | | AGATGACAGAAGAGGAGCAACCTGGAAAGTTGCCCGGAACCCATTAATGAGC | |
| | | | CCTATTTGTGGTATATGGTTTGCCTTTAGGGCAGAAATAATCTTGGCTTGAGAA | |
| | | | CATGGAAAGGGGAGGGAAGACGGGCTATTGTTAGTCCTGTGAGTCATAGGAT | |
| | | | GATAAAAATAGGACAAATATCGAAAGAAGGATGAATCTCAGCTCAGCACGGG | |
| | | | CGAATTTGGCTGCTCTAGTGCACCGGTCTCCCGTAGGGGAATAACAAAAATGA | |
| | | | AAACATTCCGAAACACTGATGAAATTCTTGATGCAGTTTTTAAAACTTCTTGGA | |
| | | | GACAG | |
| tRNA-Tyr- | 1047.01 | 90.93 | AGCGCTCCGGTTTTTCTGTGCTGAACCTCAGGGGACGCCGACACACGTACACG | 91 |
| GTA-5-1 | | | TC | |

Note:
The last row is original 55 bp 5′ leader sequence.
*Average normalized suppression ratio = (PTC-NanoLuciferase luminescence [+ACE-tRNA]/Firefly luminescence)/(PTC-Nanoluciferase luminescence [no ACE-tRNA]/Firefly luminescence)

### Example 6

Assays were carried out to identify 3′ trailers in a similar manner. Briefly, 3′ processing (256 members) and long 3′ trailer (389 members) libraries were ordered and cloned as outlined above into the 3′ trailer screening plasmid (FIG. 2C). The 3′ trailer libraries were transformed, prepared, transfected, and assayed for PTC suppression as outlined above. Average normalized suppression ratio was obtained according to the following:

$$Average normalized suppression ratio = ( PTC - NanoLuciferase luminescence [ + ACE - tRNA ] / Firefly luminescence ) / ( PTC - Nanoluciferase luminescence [ no ACE - tRNA ] / Firefly luminescence ) .$$

The results of those exhibiting ratios higher than that of the original one are shown in Tables 2A and 2B.

TABLE 2A
Results of 4-bp 3′ trailer screen for ArgTGA in HEK293T cells

| | | Average | | |
|---|---|---|---|---|
| | | Normalized | | |
| | | Suppression | | SEQ ID |
| Name | Sequence | Ratio | S.E.M. | NO: |
| GACC | AGCGCTCCGGTTTTTCTGTGCTGAACCTCAGGGGACGCCGACACACGTACA | 884.49 | 14.94 | 92 |
| 4 bp | CGTCGGCTCTGTGGCGCAATGGATAGCGCATTGGACTTCAAATTCAAAGG | | | |
| trailer | TTGTGGGTTCGAGTCCCACCAGAGTCGGACCTTTTTTT | | | |
| GGTA | AGCGCTCCGGTTTTTCTGTGCTGAACCTCAGGGGACGCCGACACACGTACA | 844.83 | 35.48 | 93 |
| 4 bp | CGTCGGCTCTGTGGCGCAATGGATAGCGCATTGGACTTCAAATTCAAAGG | | | |
| trailer | TTGTGGGTTCGAGTCCCACCAGAGTCGGGTATTTTTTT | | | |
| GTCA | AGCGCTCCGGTTTTTCTGTGCTGAACCTCAGGGGACGCCGACACACGTACA | 826.45 | 41.98 | 94 |
| 4 bp | CGTCGGCTCTGTGGCGCAATGGATAGCGCATTGGACTTCAAATTCAAAGG | | | |
| trailer | TTGTGGGTTCGAGTCCCACCAGAGTCGGTCATTTTTTT | | | |
| AGGC | AGCGCTCCGGTTTTTCTGTGCTGAACCTCAGGGGACGCCGACACACGTACA | 806.57 | 29.59 | 95 |
| 4 bp | CGTCGGCTCTGTGGCGCAATGGATAGCGCATTGGACTTCAAATTCAAAGG | | | |
| trailer | TTGTGGGTTCGAGTCCCACCAGAGTCGAGGCTTTTTTT | | | |
| GTCC | AGCGCTCCGGTTTTTCTGTGCTGAACCTCAGGGGACGCCGACACACGTACA | 777.17 | 20.81 | 96 |
| 4 bp | CGTCGGCTCTGTGGCGCAATGGATAGCGCATTGGACTTCAAATTCAAAGG | | | |
| trailer | TTGTGGGTTCGAGTCCCACCAGAGTCGGTCCTTTTTTT | | | |

Note:
1. The last row contains the original 4 bp 3′ trailer sequence.
2. o5′ leader sequence-oArgTGA-3′ trailer sequence, (o prefix denotes original).

TABLE 2B
Results of 4-bp 3′ trailer screen for ArgTGA in 16HBE14o-cells

| | | Average | | |
|---|---|---|---|---|
| | | Normalized | | |
| | | Suppression | | SEQ ID |
| Name | Sequence | Ratio | S.E.M. | NO: |
| GACC 4 bp | AGCGCTCCGGTTTTTCTGTGCTGAACCTCAGGGGACGCCGACACACGTA | 137.55 | 9.17 | 97 |
| trailer | CACGTCGGCTCTGTGGCGCAATGGATAGCGCATTGGACTTCAAATTCAA | | | |
| | AGGTTGTGGGTTCGAGTCCCACCAGAGTCGGACCTTTTTTT | | | |
| GGTA 4 bp | AGCGCTCCGGTTTTTCTGTGCTGAACCTCAGGGGACGCCGACACACGTA | 111.11 | 6.15 | 98 |
| trailer | CACGTCGGCTCTGTGGCGCAATGGATAGCGCATTGGACTTCAAATTCAA | | | |
| | AGGTTGTGGGTTCGAGTCCCACCAGAGTCGGGTATTTTTTT | | | |
| AAGC 4 bp | AGCGCTCCGGTTTTTCTGTGCTGAACCTCAGGGGACGCCGACACACGTA | 111.05 | 12.34 | 99 |
| trailer | CACGTCGGCTCTGTGGCGCAATGGATAGCGCATTGGACTTCAAATTCAA | | | |
| | AGGTTGTGGGTTCGAGTCCCACCAGAGTCGAAGCTTTTTTT | | | |
| AAAG 4 bp | AGCGCTCCGGTTTTTCTGTGCTGAACCTCAGGGGACGCCGACACACGTA | 110.12 | 6.83 | 100 |
| trailer | CACGTCGGCTCTGTGGCGCAATGGATAGCGCATTGGACTTCAAATTCAA | | | |
| | AGGTTGTGGGTTCGAGTCCCACCAGAGTCGAAAGTTTTTTT | | | |
| CTTT 4 bp | AGCGCTCCGGTTTTTCTGTGCTGAACCTCAGGGGACGCCGACACACGTA | 109.91 | 5.98 | 101 |
| trailer | CACGTCGGCTCTGTGGCGCAATGGATAGCGCATTGGACTTCAAATTCAA | | | |
| | AGGTTGTGGGTTCGAGTCCCACCAGAGTCGCTTTTTTTTTT | | | |
| AATG 4 bp | AGCGCTCCGGTTTTTCTGTGCTGAACCTCAGGGGACGCCGACACACGTA | 107.12 | 11.12 | 102 |
| trailer | CACGTCGGCTCTGTGGCGCAATGGATAGCGCATTGGACTTCAAATTCAA | | | |
| | AGGTTGTGGGTTCGAGTCCCACCAGAGTCGAATGTTTTTTT | | | |
| CTAT 4 bp | AGCGCTCCGGTTTTTCTGTGCTGAACCTCAGGGGACGCCGACACACGTA | 107.05 | 9.19 | 103 |
| trailer | CACGTCGGCTCTGTGGCGCAATGGATAGCGCATTGGACTTCAAATTCAA | | | |
| | AGGTTGTGGGTTCGAGTCCCACCAGAGTCGCTATTTTTTTT | | | |
| GGAT 4 bp | AGCGCTCCGGTTTTTCTGTGCTGAACCTCAGGGGACGCCGACACACGTA | 105.49 | 7.37 | 104 |
| trailer | CACGTCGGCTCTGTGGCGCAATGGATAGCGCATTGGACTTCAAATTCAA | | | |
| | AGGTTGTGGGTTCGAGTCCCACCAGAGTCGGGATTTTTTTT | | | |
| AGCT 4 bp | AGCGCTCCGGTTTTTCTGTGCTGAACCTCAGGGGACGCCGACACACGTA | 104.85 | 9.22 | 105 |
| trailer | CACGTCGGCTCTGTGGCGCAATGGATAGCGCATTGGACTTCAAATTCAA | | | |
| | AGGTTGTGGGTTCGAGTCCCACCAGAGTCGAGCTTTTTTTT | | | |
| AGAC 4 bp | AGCGCTCCGGTTTTTCTGTGCTGAACCTCAGGGGACGCCGACACACGTA | 103.50 | 10.73 | 106 |
| trailer | CACGTCGGCTCTGTGGCGCAATGGATAGCGCATTGGACTTCAAATTCAA | | | |
| | AGGTTGTGGGTTCGAGTCCCACCAGAGTCGAGACTTTTTTT | | | |
| GTCC 4 bp | AGCGCTCCGGTTTTTCTGTGCTGAACCTCAGGGGACGCCGACACACGTA | 103.15 | 5.88 | 107 |
| trailer | CACGTCGGCTCTGTGGCGCAATGGATAGCGCATTGGACTTCAAATTCAA | | | |
| | AGGTTGTGGGTTCGAGTCCCACCAGAGTCGGTCCTTTTTTT | | | |

Note:
1. The last row contains the original 4 bp 3′ trailer sequence.
2. o5′ leader sequence-oArgTGA-3′ trailer sequence, (o prefix denotes original).

### Example 7

Assays were carried to out to identify t-stem mutants and sticky stem mutants that result in enhanced tRNA expression or nonsense mutation suppression in the manner described above. Briefly, ArgTGA t-stem (28 members), ArgTGA sticky stem (128 members), LeuTGA t-stem (28 members), LeuTGA sticky stem (128 members), and GlyTGA t-stem (28 members) libraries were ordered as pairs of single stranded DNA oligos, annealed and cloned via a high throughput Golden Gate cloning strategy using BbsI restriction enzyme into the ACE-tRNA screening plasmid (FIG. 2B). The ACE-tRNA libraries were transformed, prepared, transfected into 16HBE14o− cells, and assayed for PTC suppression as outlined above. Average normalized suppression ratio was obtained according to the following:

$$Average normalized suppression ratio = ( PTC - NanoLuciferase luminescence [ + ACE - tRNA ] / Firefly luminescence ) / ( PTC - Nanoluciferase luminescence [ no ACE - tRNA ] / Firefly luminescence )$$

The results of those exhibiting ratios higher than that of the original one are shown in Tables 3-7 except that, with regard to Table 5, the T-stem sequence of the original GlyTGA was the best t-stem among those tested.

TABLE 3
Results of ArgTGA t-stem screen in 16HBE14o-cells

| | | Average | | |
|---|---|---|---|---|
| | | Normalized | | |
| | | Suppression | | SEQ ID |
| Name | Sequence | Ratio | S.E.M. | NO: |
| ArgTGA_ | AGCGCTCCGGTTTTTCTGTGCTGAACCTCAGGGGACGCCGACACACG | 269.97 | 9.77 | 108 |
| ts5 | TACACGTCGGCTCTGTGGCGCAATGGATAGCGCATTGGACTTCAAAT | | | |
| | TCAAAGGTTGCGGGTTCGAGTCCCGCCAGAGTCGGTCCTTTTTTT | | | |
| ArgTGA_ | AGCGCTCCGGTTTTTCTGTGCTGAACCTCAGGGGACGCCGACACACG | 256.06 | 13.96 | 109 |
| ts9 | TACACGTCGGCTCTGTGGCGCAATGGATAGCGCATTGGACTTCAAAT | | | |
| | TCAAAGGTTCCGGGTTCGAGTCCCGGCAGAGTCGGTCCTTTTTTT | | | |
| ArgTGA_ | AGCGCTCCGGTTTTTCTGTGCTGAACCTCAGGGGACGCCGACACACG | 245.77 | 8.80 | 110 |
| ts10 | TACACGTCGGCTCTGTGGCGCAATGGATAGCGCATTGGACTTCAAAT | | | |
| | TCAAAGGTTCCCGGTTCGAGTCCGGGCAGAGTCGGTCCTTTTTTT | | | |
| ArgTGA_ | AGCGCTCCGGTTTTTCTGTGCTGAACCTCAGGGGACGCCGACACACG | 230.93 | 9.13 | 111 |
| ts2 | TACACGTCGGCTCTGTGGCGCAATGGATAGCGCATTGGACTTCAAAT | | | |
| | TCAAAGGTTACGGGTTCGAGTCCCGTCAGAGTCGGTCCTTTTTTT | | | |
| ArgTGA_ | AGCGCTCCGGTTTTTCTGTGCTGAACCTCAGGGGACGCCGACACACG | 215.17 | 4.95 | 112 |
| ts_ | TACACGTCGGCTCTGTGGCGCAATGGATAGCGCATTGGACTTCAAAT | | | |
| oArgTGA | TCAAAGGTTGTGGGTTCGAGTCCCACCAGAGTCGGTCCTTTTTTT | | | |

Note:
1. The last row contains the original T-stem sequence
2. o5′ leader sequence-ArgTGA t-stem mutant-o3′ trailer sequence, (o prefix denotes original).

TABLE 4
Results of LeuTGA t-stem screen in 16HBE14o-cells

| | | Average | | |
|---|---|---|---|---|
| | | Normalized | | |
| | | Suppression | | SEQ ID |
| Name | Sequence | Ratio | S.E.M. | NO: |
| LeuTGA_ | AGCGCTCCGGTTTTTCTGTGCTGAACCTCAGGGGACGCCGACACACG | 229.36 | 13.95 | 113 |
| ts9 | TACACGTCACCAGAATGGCCGAGTGGTtAAGGCGTTGGACTtcaGAT | | | |
| | CCAATGGATTCATATCCGCCCGGGTTCGAACCCCGGTTCTGGTAGTC | | | |
| | CTTTTTTT | | | |
| LeuTGA_ | AGCGCTCCGGTTTTTCTGTGCTGAACCTCAGGGGACGCCGACACACG | 88.09 | 9.04 | 114 |
| ts2 | TACACGTCACCAGAATGGCCGAGTGGTtAAGGCGTTGGACTtcaGAT | | | |
| | CCAATGGATTCATATCCGCACGGGTTCGAACCCCGTTTCTGGTAGTC | | | |
| | CTTTTTTT | | | |
| LeuTGA_ | AGCGCTCCGGTTTTTCTGTGCTGAACCTCAGGGGACGCCGACACACG | 86.03 | 8.93 | 115 |
| ts21 | TACACGTCACCAGAATGGCCGAGTGGTtAAGGCGTTGGACTtcaGAT | | | |
| | CCAATGGATTCATATCCGCGCGGGTTCGAACCCTGCTTCTGGTAGTC | | | |
| | CTTTTTTT | | | |
| LeuTGA_ | AGCGCTCCGGTTTTTCTGTGCTGAACCTCAGGGGACGCCGACACACG | 83.47 | 4.34 | 116 |
| ts_ | TACACGTCACCAGAATGGCCGAGTGGTtAAGGCGTTGGACTtcaGAT | | | |
| oLeuTGA | CCAATGGATTCATATCCGCGTGGGTTCGAACCCCACTTCTGGTAGTC | | | |
| | CTTTTTTT | | | |

Note:
1. The last row contains the original T-stem sequence
2. o5′ leader sequence-LeuTGA t-stem mutant-o3′ trailer sequence, (o prefix denotes original).

TABLE 5
Results of GlyTGA t-stem screen in 16HBE14o-cells

| | | Average | | |
|---|---|---|---|---|
| | | Normalized | | |
| | | Suppression | | SEQ ID |
| Name | Sequence | Ratio | S.E.M. | NO: |
| GlyTGA_ | AGCGCTCCGGTTTTTCTGTGCTGAACCTCAGGGGACGCCGACACAC | 67.44 | 2.03 | 117 |
| ts_ | GTACACGTCGCGTTGGTGGTATAGTGGTTAGCATAGCTGCCTtcaA | | | |
| oGlyTGA | AGCAGTTGACCCGGGTTCGATTCCCGGCCAACGCAGTCCTTTTTTT | | | |

Note:
1. The row contains the original T-stem sequence
2. o5′ leader sequence-GlyTGA t-stem mutant-o3′ trailer sequence, (o prefix denotes original).

TABLE 6
Results of ArgTGA sticky stems screen in 16HBE14o-cells

| | | Average | | |
|---|---|---|---|---|
| | | Normalized | | |
| | | Suppression | | SEQ ID |
| Name | Sequence | Ratio | S.E.M. | NO: |
| ArgTGA_ | AGCGCTCCGGTTTTTCTGTGCTGAACCTCAGGGGACGCCGACACACGT | 341.60 | 11.52 | 118 |
| SSlib99 | ACACGTCGCCCCTGTGGCGCAATGGATAGCGCATTGGACTtcaAATCCA | | | |
| | AAGGTTGTGGGTTCGAGTCCCACCAGGGGCGGTCCTTTTTTT | | | |
| ArgTGA_ | AGCGCTCCGGTTTTTCTGTGCTGAACCTCAGGGGACGCCGACACACGT | 306.60 | 16.85 | 119 |
| SSlib49 | ACACGTCGGCCCCGTGGCGCAATGGATAGCGCATTGGACTtcaAATTC | | | |
| | AAAGGTTGTGGGTTCGAGTCCCACCGGGGTCGGTCCTTTTTTT | | | |
| ArgTGA_ | AGCGCTCCGGTTTTTCTGTGCTGAACCTCAGGGGACGCCGACACACGT | 298.13 | 20.28 | 120 |
| SSlib97 | ACACGTCGCCCCTGTGGCGCAATGGATAGCGCATTGGACTtcaAATTCA | | | |
| | AAGGTTGTGGGTTCGAGTCCCACCAGGGGCGGTCCTTTTTTT | | | |
| ArgTGA_ | AGCGCTCCGGTTTTTCTGTGCTGAACCTCAGGGGACGCCGACACACGT | 298.09 | 15.57 | 121 |
| SSlib37 | ACACGTCGGCCCTGTGGCGCAATGGATAGCGCATCGGACTtcaAATTC | | | |
| | GAAGGTTGTGGGTTCGAGTCCCACCAGGGTCGGTCCTTTTTTT | | | |
| ArgTGA_ | AGCGCTCCGGTTTTTCTGTGCTGAACCTCAGGGGACGCCGACACACGT | 288.84 | 23.57 | 122 |
| SSlib33 | ACACGTCGGCCCTGTGGCGCAATGGATAGCGCATTGGACTtcaAATTCA | | | |
| | AAGGTTGTGGGTTCGAGTCCCACCAGGGTCGGTCCTTTTTTT | | | |
| ArgTGA_ | AGCGCTCCGGTTTTTCTGTGCTGAACCTCAGGGGACGCCGACACACGT | 286.23 | 20.10 | 123 |
| SSlib85 | ACACGTCGCCTCCGTGGCGCAATGGATAGCGCATCGGACTtcaAATTCG | | | |
| | AAGGTTGTGGGTTCGAGTCCCACCGGAGGCGGTCCTTTTTTT | | | |
| ArgTGA_ | AGCGCTCCGGTTTTTCTGTGCTGAACCTCAGGGGACGCCGACACACGT | 282.50 | 15.05 | 124 |
| SSlib117 | ACACGTCGCCCCCGTGGCGCAATGGATAGCGCATCGGACTtcaAATTC | | | |
| | GAAGGTTGTGGGTTCGAGTCCCACCGGGGGGGGTCCTTTTTTT | | | |
| ArgTGA_ | AGCGCTCCGGTTTTTCTGTGCTGAACCTCAGGGGACGCCGACACACGT | 281.96 | 9.48 | 125 |
| SSlib104 | ACACGTCGCCCCTGTGGCGCAATGGATAGCGCATCGGCCTtcaAAGCC | | | |
| | GAAGGTTGTGGGTTCGAGTCCCACCAGGGGCGGTCCTTTTTTT | | | |
| ArgTGA_ | AGCGCTCCGGTTTTTCTGTGCTGAACCTCAGGGGACGCCGACACACGT | 269.89 | 3.88 | 126 |
| SSlib103 | ACACGTCGCCCCTGTGGCGCAATGGATAGCGCATCGGACTtcaAATCC | | | |
| | GAAGGTTGTGGGTTCGAGTCCCACCAGGGGCGGTCCTTTTTTT | | | |
| ArgTGA_ | AGCGCTCCGGTTTTTCTGTGCTGAACCTCAGGGGACGCCGACACACGT | 264.37 | 5.77 | 127 |
| SSlib115 | ACACGTCGCCCCCGTGGCGCAATGGATAGCGCATTGGACTtcaAATCCA | | | |
| | AAGGTTGTGGGTTCGAGTCCCACCGGGGGGGGTCCTTTTTTT | | | |
| ArgTGA_ | AGCGCTCCGGTTTTTCTGTGCTGAACCTCAGGGGACGCCGACACACGT | 263.03 | 7.30 | 128 |
| SSlib63 | ACACGTCGGCCCCGTGGCGCAATGGATAGCGCACCGGACTtcaAATCC | | | |
| | GGAGGTTGTGGGTTCGAGTCCCACCGGGGTCGGTCCTTTTTTT | | | |
| ArgTGA_ | AGCGCTCCGGTTTTTCTGTGCTGAACCTCAGGGGACGCCGACACACGT | 256.21 | 19.85 | 129 |
| SSlib109 | ACACGTCGCCCCTGTGGCGCAATGGATAGCGCACCGGACTtcaAATTC | | | |
| | GGAGGTTGTGGGTTCGAGTCCCACCAGGGGCGGTCCTTTTTTT | | | |
| ArgTGA_ | AGCGCTCCGGTTTTTCTGTGCTGAACCTCAGGGGACGCCGACACACGT | 255.52 | 17.21 | 130 |
| SSlib36 | ACACGTCGGCCCTGTGGCGCAATGGATAGCGCATTGGCCTtcaAAGCC | | | |
| | AAAGGTTGTGGGTTCGAGTCCCACCAGGGTCGGTCCTTTTTTT | | | |
| ArgTGA_ | AGCGCTCCGGTTTTTCTGTGCTGAACCTCAGGGGACGCCGACACACGT | 249.33 | 24.41 | 131 |
| SSlib83 | ACACGTCGCCTCCGTGGCGCAATGGATAGCGCATTGGACTtcaAATCCA | | | |
| | AAGGTTGTGGGTTCGAGTCCCACCGGAGGCGGTCCTTTTTTT | | | |
| ArgTGA_ | AGCGCTCCGGTTTTTCTGTGCTGAACCTCAGGGGACGCCGACACACGT | 215.00 | 11.15 | 132 |
| SSlib_ | ACACGTCGGCTCTGTGGCGCAATGGATAGCGCATTGGACTtcaAATTCA | | | |
| oArgTGA | AAGGTTGTGGGTTCGAGTCCCACCAGAGTCGGTCCTTTTTTT | | | |

Note:
1. The last row contains the original ArgTGA sequence.
2. o5′ leader sequence-ArgTGA sticky stem mutant-o3′ trailer sequence, (o prefix denotes original).

TABLE 7
Results of LeuTGA sticky stems screen in 16HBE14o-cells

| | | Average | | |
|---|---|---|---|---|
| | | Normalized | | |
| | | Suppression | | SEQ ID |
| Name | Sequence | Ratio | S.E.M. | NO: |
| LeuTGA_ | AGCGCTCCGGTTTTTCTGTGCTGAACCTCAGGGGACGCCGACACACGTA | 212.89 | 16.38 | 133 |
| SSlib94 | CACGTCGCCAGGATGGCCGAGTGGTTAAGGCGCCGGACTtcaGATCCGG | | | |
| | TGGACTCATGTCCGCGTGGGTTCGAACCCCACTCCTGGCAGTCCTTTTTT | | | |
| | T | | | |
| LeuTGA_ | AGCGCTCCGGTTTTTCTGTGCTGAACCTCAGGGGACGCCGACACACGTA | 208.48 | 10.66 | 134 |
| SSlib93 | CACGTCGCCAGGATGGCCGAGTGGTTAAGGCGCCGGACTtcaGATCCGG | | | |
| | TGGATTCATATCCGCGTGGGTTCGAACCCCACTCCTGGCAGTCCTTTTTT | | | |
| | T | | | |
| LeuTGA_ | AGCGCTCCGGTTTTTCTGTGCTGAACCTCAGGGGACGCCGACACACGTA | 165.25 | 15.53 | 135 |
| SSlib89 | CACGTCGCCAGGATGGCCGAGTGGTTAAGGCGCTGGACTtcaGATCCAG | | | |
| | TGGATTCATATCCGCGTGGGTTCGAACCCCACTCCTGGCAGTCCTTTTTT | | | |
| | T | | | |
| LeuTGA_ | AGCGCTCCGGTTTTTCTGTGCTGAACCTCAGGGGACGCCGACACACGTA | 163.84 | 8.88 | 136 |
| SSlib117 | CACGTCGCCGGGATGGCCGAGTGGTTAAGGCGTCGGACTtcaGATCCGA | | | |
| | TGGATTCATATCCGCGTGGGTTCGAACCCCACTCCCGGCAGTCCTTTTTT | | | |
| | T | | | |
| LeuTGA_ | AGCGCTCCGGTTTTTCTGTGCTGAACCTCAGGGGACGCCGACACACGTA | 147.92 | 15.63 | 137 |
| SSlib86 | CACGTCGCCAGGATGGCCGAGTGGTTAAGGCGTCGGACTtcaGATCCGA | | | |
| | TGGACTCATGTCCGCGTGGGTTCGAACCCCACTCCTGGCAGTCCTTTTTT | | | |
| | T | | | |
| LeuTGA_ | AGCGCTCCGGTTTTTCTGTGCTGAACCTCAGGGGACGCCGACACACGTA | 138.42 | 14.07 | 138 |
| SSlib81 | CACGTCGCCAGGATGGCCGAGTGGTTAAGGCGTTGGACTtcaGATCCAA | | | |
| | TGGATTCATATCCGCGTGGGTTCGAACCCCACTCCTGGCAGTCCTTTTTT | | | |
| | T | | | |
| LeuTGA_ | AGCGCTCCGGTTTTTCTGTGCTGAACCTCAGGGGACGCCGACACACGTA | 123.36 | 15.09 | 139 |
| SSlib82 | CACGTCGCCAGGATGGCCGAGTGGTTAAGGCGTTGGACTtcaGATCCAA | | | |
| | TGGACTCATGTCCGCGTGGGTTCGAACCCCACTCCTGGCAGTCCTTTTTT | | | |
| | T | | | |
| LeuTGA_ | AGCGCTCCGGTTTTTCTGTGCTGAACCTCAGGGGACGCCGACACACGTA | 122.59 | 6.99 | 140 |
| SSlib121 | CACGTCGCCGGGATGGCCGAGTGGTTAAGGCGCTGGACTtcaGATCCAG | | | |
| | TGGATTCATATCCGCGTGGGTTCGAACCCCACTCCCGGCAGTCCTTTTTT | | | |
| | T | | | |
| LeuTGA_ | AGCGCTCCGGTTTTTCTGTGCTGAACCTCAGGGGACGCCGACACACGTA | 121.17 | 12.94 | 141 |
| SSlib87 | CACGTCGCCAGGATGGCCGAGTGGTTAAGGCGTCGGACTtcaGATCCGA | | | |
| | TGGCTTCATAGCCGCGTGGGTTCGAACCCCACTCCTGGCAGTCCTTTTTT | | | |
| | T | | | |
| LeuTGA_ | AGCGCTCCGGTTTTTCTGTGCTGAACCTCAGGGGACGCCGACACACGTA | 106.42 | 3.51 | 142 |
| SSlib125 | CACGTCGCCGGGATGGCCGAGTGGTTAAGGCGCCGGACTtcaGATCCGG | | | |
| | TGGATTCATATCCGCGTGGGTTCGAACCCCACTCCCGGCAGTCCTTTTTT | | | |
| | T | | | |
| LeuTGA_ | AGCGCTCCGGTTTTTCTGTGCTGAACCTCAGGGGACGCCGACACACGTA | 105.22 | 7.44 | 143 |
| SSlib2 | CACGTCACCAGAATGGCCGAGTGGTTAAGGCGTTGGACTtcaGATCCAAT | | | |
| | GGACTCATGTCCGCGTGGGTTCGAACCCCACTTCTGGTAGTCCTTTTTTT | | | |
| LeuTGA_ | AGCGCTCCGGTTTTTCTGTGCTGAACCTCAGGGGACGCCGACACACGTA | 101.42 | 5.55 | 144 |
| SSlib118 | CACGTCGCCGGGATGGCCGAGTGGTTAAGGCGTCGGACTtcaGATCCGA | | | |
| | TGGACTCATGTCCGCGTGGGTTCGAACCCCACTCCCGGCAGTCCTTTTTT | | | |
| | T | | | |
| LeuTGA_ | AGCGCTCCGGTTTTTCTGTGCTGAACCTCAGGGGACGCCGACACACGTA | 97.69 | 9.19 | 145 |
| SSlib90 | CACGTCGCCAGGATGGCCGAGTGGTTAAGGCGCTGGACTtcaGATCCAG | | | |
| | TGGACTCATGTCCGCGTGGGTTCGAACCCCACTCCTGGCAGTCCTTTTTT | | | |
| | T | | | |
| LeuTGA_ | AGCGCTCCGGTTTTTCTGTGCTGAACCTCAGGGGACGCCGACACACGTA | 87.27 | 11.47 | 146 |
| SSlib83 | CACGTCGCCAGGATGGCCGAGTGGTTAAGGCGTTGGACTtcaGATCCAA | | | |
| | TGGCTTCATAGCCGCGTGGGTTCGAACCCCACTCCTGGCAGTCCTTTTTT | | | |
| | T | | | |
| LeuTGA_ | AGCGCTCCGGTTTTTCTGTGCTGAACCTCAGGGGACGCCGACACACGTA | 82.54 | 5.39 | 147 |
| SSlib_ | CACGTCACCAGAATGGCCGAGTGGTTAAGGCGTTGGACTtcaGATCCAAT | | | |
| oLeuTGA | GGATTCATATCCGCGTGGGTTCGAACCCCACTTCTGGTAGTCCTTTTTTT | | | |

Note:
1. The last row contains the original LeuTGA sequence.
2. o5′ leader sequence-LeuTGA sticky stem mutant-o3′ trailer sequence, (o prefix denotes original).

### Example 8

Assays were carried to out to compare effects of U6 and H1 promoters with that of the original 5′ leader in the manner described above. The results are shown in Table 8. The results indicate that the original 5′ leader is comparable to or better than U6 and H1 promoters. The results also indicate that the 5′ leader of tRNA-Cys-GCA-12-1 are much better than U6 and H1 promoters as well as the original 5′ leader. It follows that this 5′ leader segment and the other 5′ leader segments described herein may be used as an enhancing element to enhance expression of a gene such as a tRNA gene.

TABLE 8
Results of comparing U6 and H1 promoters to the original 5′ leader

| | | Average | | |
|---|---|---|---|---|
| | | Normalized | | |
| | | Suppression | | SEQ ID |
| 5′ leader | Sequence | Ratio | S.E.M. | NO |
| tRNA-Tyr- | AGCGCTCCGGTTTTTCTGTGCTGAACCTCAGGGGACGCCGACA | 224.96 | 3.33 | 148 |
| GTA-5-1 | CACGTACACGTC | | | |
| U6 | Gagggcctatttcccatgattccttcatatttgcatatacgat | 220.15 | 9.31 | 149 |
| promoter | acaaggctgttagagagataattggaattaatttgactgtaaa | | | |
| | cacaaagatattagtacaaaatacgtgacgtagaaagtaataa | | | |
| | tttcttgggtagtttgcagttttaaaattatgttttaaaatgg | | | |
| | actatcatatgcttaccgtaacttgaaagtatttcgatttctt | | | |
| | ggctttatatatcttgtggaaaggacgaaacac | | | |
| H1 | Gaacgctgacgtcatcaacccgctccaaggaatcgcgggccca | 248.76 | 5.67 | 150 |
| promoter | gtgtcactaggcgggaacacccagcgcgcgtgcgccctggcag | | | |
| | gaagatggctgtgagggacaggggagtggcgccctgcaatatt | | | |
| | tgcatgtcgctatgtgttctgggaaatcaccataaacgtgaaa | | | |
| | tgtctttggatttgggaatcttataagttctgtatgagaccac | | | |
| | gaaacac | | | |
| tRNA-Cys- | GCCAGGGGTGTGGCCATACAGGTTTATAGTGGTTAGTAGAGAC | 437.39 | 20.57 | 151 |
| GCA-12-1 | AAGTAAGTGGGT | | | |

### Example 9

In this example, assays were carried out to examine tRNA genes, each of which was appended with a “tRNA transcript tabulator” or “tRNA transcript counter.” Shown in FIG. 8A is such a tRNA genes with a transcript tabulator.

As shown in FIG. 8, appending a tRNA transcript tabulator to the 3′ end of ACE-tRNAs allowed one to quantify ACE-tRNA-dependent expression of an RNA target by RT-qPCR. Transcription of a sequence without an RNA pol III promoter appended to the 3′ end of the ACE-tRNA upstream of the p (T) transcription terminator is dependent upon transcription of the ACE-tRNA. This is denoted as a ‘tRNA transcript tabulator’ (TTT) within the context of a tRNA gene (FIG. 8A). A self-cleaving ribozyme was chosen as the TTT, as it self-cleaves itself from the tRNA following transcription. The ACE-tRNA and ribozyme TTT are transcribed as a single transcript before the ribozyme cleaves the backbone (cleavage site denoted with an arrow, FIG. 8B). ACE-tRNAs are not amenable to direct quantification via real-time reverse transcription quantitative PCR (RT-qPCR) without extensive processing as they contain many modified nucleotides which inhibit reverse transcriptase (FIG. 8C). As the TTT abundance is directly proportional to the ACE-tRNA abundance and the TTT does not contain modifications which inhibit reverse transcriptase, quantification of the tRNA transcript counter acts as a surrogate for quantification of the ACE-tRNA. The TTT sequence was cloned downstream of each of the ACE-tRNA sequences as denoted in FIG. 8D-G. Depending on the ACE-tRNA any of the 5′-UCE, t-stem, or sticky stem sequences were varied. Each construct was transfected into 16HBE14o− cells using Lipofectamine LTX with PLUS reagent (Thermo Fisher Scientific) as per manufacturer instruction. The cells were harvested in Buffer RLT (Qiagen) and total RNA was purified. The total RNA was subjected to relative quantification by RT-qPCR with probes directed against the TTT and TATA-binding protein (TBP, endogenous control transcript). The amount of TTT relative to TBP for each ACE-tRNA sequence was obtained for ACE-tRNA^{Arg}_{TGA} (FIG. 8D), ACE-tRNA^{Leu}_{TGA} (FIG. 8E), ACE-tRNA^{Gly}_{TGA} (FIG. 8F), and ACE-tRNA^{Trp}_{TGA} (FIG. 8G).

### Example 10

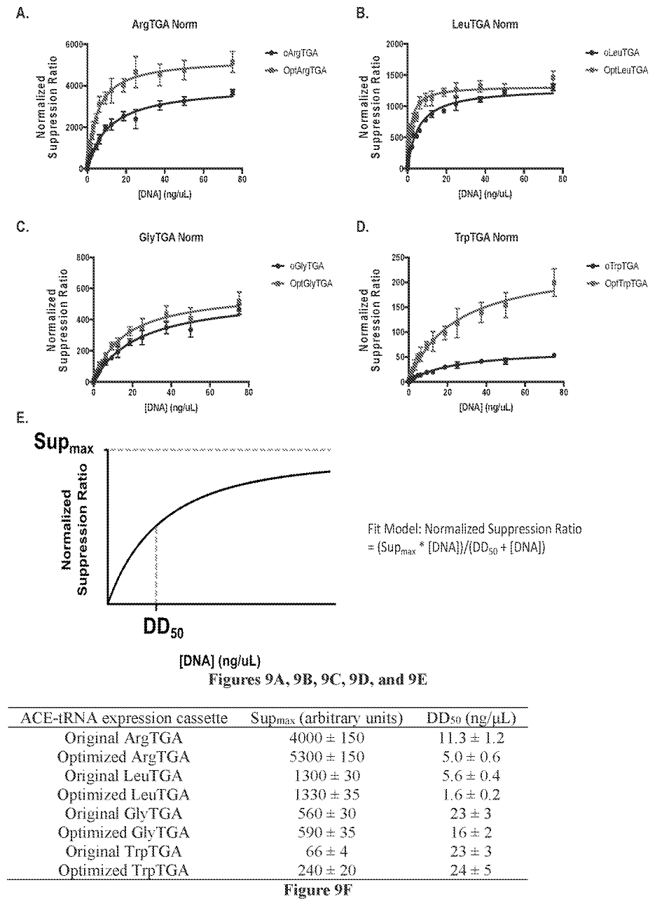

Additional assays were carried out to identify mutants having combinations of two or more of the above described optimal 5′- and 3′-flanking sequences, anticodon loops, and T-stem sequences. The mutants and results are shown in Table 9 and Table 10 below and FIG. 9.

As shown in FIG. 9, optimized ACE-tRNA expression cassettes demonstrate higher peak levels of PTC suppression (higher Supₘₐₓ) and/or display lower levels of DNA delivery needed to reach maximal PTC suppression (lower DD₅₀). Two sets of two-fold serial dilution series (starting at 75 ng/μL and 50 ng/μL respectively) of each original and optimized ACE-tRNA cassette in the pUC57 mini tRNA Dest were made and mixed with pNanoReporter 2.0 TGA (no tRNA cassette, 25 ng/μL) and transfected into HEK293T cells. The resulting data were fit to the hyperbolic model shown above using nonlinear regression (Prism 7.3) where Supₘₐₓ corresponds to the maximal level of nonsense suppression displayed by the ACE-tRNA and DD₅₀ (Delivered DNA) corresponds to the concentration of DNA (ng/μL) required for a half-maximal nonsense suppression response.

Certain optimized and original ACE-tRNA sequences in the DNA constructs used here are also shown in Table 9 below. Table 9 contains a series of sequences containing the best sequences shown in any of tables 1A, 1B, 2A, 2B, 3, 4, 5, 6, or 7 tested in combination for optimal function together in a single fully optimized ACE-tRNA sequence. In this table, original ACE-tRNA sequences are shown in italic. Top fully optimized ACE-tRNA sequences are shown in bold.

TABLE 9
Best Combos

| | | | Average | | |
|---|---|---|---|---|---|
| | | | Normalized | | |
| | | Sequence | Suppression | | SEQ |
| Name | Sequence | Description | Ratio | S.E.M. | ID |
| Top5p3p | GCCAGGGGTGTGGCCATACAGGTTTATAGTGG | Top 5′ (Cys-GCA-12- | 1641.9 | 98.6 | 152 |
| ArgTGA | TTAGTAGAGACAAGTAAGTGGGTGGCTCTGTG | 1), ArgTGA, top 3′- | | | |
| | GCGCAATGGATAGCGCATTGGACTTCAAATTCA | 4 bp (GACC) | | | |
| | AAGGTTGTGGGTTCGAGTCCCACCAGAGTCGG | | | | |
| | ACCTTTTTTT | | | | |
| 5pTyrGTA_ | AGCGCTCCGGTTTTTCTGTGCTGAACCTCAGGG | o5′ leader sequence- | 1047.0 | 33.8 | 153 |
| 5_1ArgTGA | GACGCCGACACACGTACACGTCGGCTCTGTGGC | oArgTGA-o3′ trailer | | | |
| | GCAATGGATAGCGCATTGGACTTCAAATTCAAA | sequence (serves as | | | |
| | GGTTGTGGGTTCGAGTCCCACCAGAGTCGGTCC | ArgTGA reference | | | |
| | TTTTTTT | sample) | | | |
| ArgTGA | GCCAGGGGTGTGGCCATACAGGTTTATAGTGG | Top 5′ (CysGCA-12- | 1471.3 | 86.7 | 154 |
| combo1 | TTAGTAGAGACAAGTAAGTGGGTGGCTCTGTG | 1), top ArgTGA t- | | | |
| | GCGCAATGGATAGCGCATTGGACTTCAAATTCA | stem (ts), top 3′-4 | | | |
| | AAGGTTGCGGGTTCGAGTCCCGCCAGAGTCGG | bp (GACC) | | | |
| | ACCTTTTTTT | | | | |
| ArgTGA | GCCAGGGGTGTGGCCATACAGGTTTATAGTGG | Top 5′ (CysGCA-12- | 1345.4 | 72.3 | 155 |
| combo2 | TTAGTAGAGACAAGTAAGTGGGTGCCCCTGTG | 1), top ArgTGA | | | |
| | GCGCAATGGATAGCGCATTGGACTtcaAATCCA | sticky stem (SS), | | | |
| | AAGGTTGTGGGTTCGAGTCCCACCAGGGGCGG | top 3′-4 bp (GACC) | | | |
| | ACCTTTTTTT | | | | |
| ArgTGA | GCCAGGGGTGTGGCCATACAGGTTTATAGTGG | Top 5′ (CysGCA-12- | 1492.6 | 107.9 | 156 |
| combo3 | TTAGTAGAGACAAGTAAGTGGGTGCCCCTGTG | 1), top ArgTGA ts | | | |
| | GCGCAATGGATAGCGCATTGGACTtcaAATCCA | AND top SS, top 3′- | | | |
| | AAGGTTGCGGGTTCGAGTCCCGCCAGGGGCGG | 4 bp (GACC) | | | |
| | ACCTTTTTTT | | | | |
| ArgTGA | GCCAGGGGTGTGGCCATACAGGTTTATAGTGG | Top 5′ (CysGCA-12- | 1471.1 | 58.1 | 157 |
| combo4 | TTAGTAGAGACAAGTAAGTGGGTGCCCCTGTG | 1), top ArgTGA ts | | | |
| | GCGCAATGGATAGCGCATTGGACTtcaAATCCA | AND top SS, 3′- | | | |
| | AAGGTTGCGGGTTCGAGTCCCGCCAGGGGCGC | 35 bp (Ile-TAT-1-1) | | | |
| | TTTTTTTCCCCATCAGTTTTTATAAACTTACACA | | | | |
| ArgTGA | GCCAGGGGTGTGGCCATACAGGTTTATAGTGG | Top 5′ (CysGCA-12- | 1802.1 | 21.8 | 158 |
| combo5 | TTAGTAGAGACAAGTAAGTGGGTGCCCCTGTG | 1), top ArgTGA ts | | | |
| | GCGCAATGGATAGCGCATTGGACTtcaAATCCA | AND top SS, 3′- | | | |
| | AAGGTTGCGGGTTCGAGTCCCGCCAGGGGCGG | 35 bp (Tyr-GTA-6-1) | | | |
| | TTCCATTTTTAAAAGTGTCTCTTCTGGGCCTGAA | | | | |
| ArgTGA | GCCAGGGGTGTGGCCATACAGGTTTATAGTGG | Top 5′ (CysGCA-12- | 1821.0 | 68.5 | 159 |
| combo6 | TTAGTAGAGACAAGTAAGTGGGTGCCCCTGTG | 1), top ArgTGA ts | | | |
| | GCGCAATGGATAGCGCATTGGACTtcaAATCCA | AND top SS, 3′- | | | |
| | AAGGTTGCGGGTTCGAGTCCCGCCAGGGGCGG | 35 bp (Ile-TAT-1-1, | | | |
| | ACCCTTTTTTTCCCCATCAGTTTTTATAAACTTA | insert GACC) | | | |
| | CACA | | | | |
| ArgTGA | GCCAGGGGTGTGGCCATACAGGTTTATAGTGG | Top 5′ (CysGCA-12- | 1833.7 | 66.0 | 160 |
| combo7 | TTAGTAGAGACAAGTAAGTGGGTGCCCCTGTG | 1), top ArgTGA ts | | | |
| | GCGCAATGGATAGCGCATTGGACTtcaAATCCA | AND top SS, 3′- | | | |
| | AAGGTTGCGGGTTCGAGTCCCGCCAGGGGCGG | 35 bp (Tyr-GTA-6-1, | | | |
| | ACCGTTCCATTTTTAAAAGTGTCTCTTCTGGGCC | insert GACC) | | | |
| | TGAA | | | | |
| ArgTGA | GCCAGGGGTGTGGCCATACAGGTTTATAGTGG | Top 5′ (CysGCA- | 1852.5 | 29.3 | 161 |
| combo8 | TTAGTAGAGACAAGTAAGTGGGTGCCCCTGTG | 12-1), top ArgTGA | | | |
| | GCGCAATGGATAGCGCATTGGACTtcaAATCCA | ts AND top SS, 3′- | | | |
| | AAGGTTGCGGGTTCGAGTCCCGCCAGGGGCG | 35bp (Ile-TAT-1-1, | | | |
| | GACCTTTTCCCCATCAGTTTTTATAAACTTACAC | replace GACC) | | | |
| | A | | | | |
| ArgTGA | GCCAGGGGTGTGGCCATACAGGTTTATAGTGG | Top 5′ (CysGCA-12- | 1650.4 | 117.1 | 162 |
| combo9 | TTAGTAGAGACAAGTAAGTGGGTGCCCCTGTG | 1), top ArgTGA ts | | | |
| | GCGCAATGGATAGCGCATTGGACTtcaAATCCA | AND top SS, 3′- | | | |
| | AAGGTTGCGGGTTCGAGTCCCGCCAGGGGCGG | 35 bp (Tyr-GTA-6-1, | | | |
| | ACCCATTTTTAAAAGTGTCTCTTCTGGGCCTGAA | replace GACC) | | | |
| 5pTyrGTA_ | AGCGCTCCGGTTTTTCTGTGCTGAACCTCAGGG | o5′ leader sequence, | 269.7 | 13.6 | 163 |
| 5_1GlyTGA | GACGCCGACACACGTACACGTCGCGTTGGTGGT | GlyTGA, o3′ trailer | | | |
| | ATAGTGGTtAGCATAGCTGCCTtcaAAGCAGTTG | sequence (serves as | | | |
| | aCCCGGGTTCGATTCCCGGCCAACGCAGTCCTTT | GlyTGA reference | | | |
| | TTTT | sample) | | | |
| 5pAlaTGC_ | TGTCCTGAGTTAAGCGACCAATAGGGACCATCT | Screen (Top 5) 5′ | 378.0 | 12.9 | 164 |
| 3_1GlyTGA | GGGAGAGCTGCTGAGAGTAGGTGCGTTGGTG | leaders, GlyTGA, o3′ | | | |
| | GTATAGTGGTtAGCATAGCTGCCTtcaAAGCAGT | trailer sequence | | | |
| | TGaCCCGGGTTCGATTCCCGGCCAACGCAGTCC | | | | |
| | TTTTTTT | | | | |
| 5pCysGCA_ | GCCAGGGGTGTGGCCATACAGGTTTATAGTGG | | 399.2 | 8.8 | 165 |
| 12_1_ | TTAGTAGAGACAAGTAAGTGGGTGCGTTGGTG | | | | |
| GlyTGA | GTATAGTGGTtAGCATAGCTGCCTtcaAAGCAGT | | | | |
| | TGaCCCGGGTTCGATTCCCGGCCAACGCAGTCC | | | | |
| | TTTTTTT | | | | |
| 5pIleTAT_ | TCAATCTGTAAGAGCATCTTCTCTTATACCTTTC | | 333.1 | 9.0 | 166 |
| 3_1GlyTGA | CAAATCCTTATTATTTCTGTGGCGTTGGTGGTAT | | | | |
| | AGTGGTtAGCATAGCTGCCTtcaAAGCAGTTGaC | | | | |
| | CCGGGTTCGATTCCCGGCCAACGCAGTCCTTTT | | | | |
| | TTT | | | | |
| 5pLeuCAG_ | ATAGCGTTTAGACAAGAGACAGAAGTGAGTCC | | 374.4 | 1.0 | 167 |
| 1_7GlyTGA | TGAGCACAATGTAGCATAAGCGCGCGTTGGTG | | | | |
| | GTATAGTGGTtAGCATAGCTGCCTtcaAAGCAGT | | | | |
| | TGaCCCGGGTTCGATTCCCGGCCAACGCAGTCC | | | | |
| | TTTTTTT | | | | |
| 5pTyrGTA_ | GATGAGGTTGCGGTAAGCATTAGAGGGCTATC | | 330.3 | 24.8 | 168 |
| 2_1GlyTGA | AGCAGCATCTTATCGCAGCGGAGGCGTTGGTG | | | | |
| | GTATAGTGGTtAGCATAGCTGCCTtcaAAGCAGT | | | | |
| | TGaCCCGGGTTCGATTCCCGGCCAACGCAGTCC | | | | |
| | TTTTTTT | | | | |
| 3pGACC | AGCGCTCCGGTTTTTCTGTGCTGAACCTCAGGG | o5′ leader sequence, | 352.9 | 1.9 | 169 |
| GlyTGA | GACGCCGACACACGTACACGTCGCGTTGGTGG | GlyTGA, top 3′-4 bp | | | |
| | TATAGTGGTtAGCATAGCTGCCTtcaAAGCAGTT | (GACC) | | | |
| | GaCCCGGGTTCGATTCCCGGCCAACGCAGACCT | | | | |
| | TTTTTT | | | | |
| Top5p3p | GCCAGGGGTGTGGCCATACAGGTTTATAGTGG | Top 5′ (Cys-GCA-12- | 368.3 | 13.0 | 170 |
| GlyTGA | TTAGTAGAGACAAGTAAGTGGGTGCGTTGGTG | 1), GlyTGA, top 3′- | | | |
| | GTATAGTGGTtAGCATAGCTGCCTtcaAAGCAGT | 4 bp (GACC) | | | |
| | TGaCCCGGGTTCGATTCCCGGCCAACGCAGACC | | | | |
| | TTTTTTT | | | | |
| GlyTGA | GCCAGGGGTGTGGCCATACAGGTTTATAGTGG | Top 5′ (CysGCA-12- | 415.6 | 7.2 | 171 |
| combo1 | TTAGTAGAGACAAGTAAGTGGGTGCGTTGGTG | 1), top GlyTGA t- | | | |
| | GTATAGTGGTTAGCATAGCTGCCTtcaAAGCAGT | stem, top 3′-4 bp | | | |
| | TGACCCCGGTTCGATTCCGGGCCAACGCAGACC | (GACC) | | | |
| | TTTTTTT | | | | |
| GlyTGA | GCCAGGGGTGTGGCCATACAGGTTTATAGTGG | (CysGCA-12-1), top | 437.9 | 0.5 | 172 |
| combo2 | TTAGTAGAGACAAGTAAGTGGGTGCGTTGGTG | GlyTGA t-stem, top | | | |
| | GTATAGTGGTTAGCATAGCTGCCTtcaAAGCAGT | 3′-35 bp (Ile-TAT-1- | | | |
| | TGACCCCGGTTCGATTCCGGGCCAACGCACTTT | 1) | | | |
| | TTTTCCCCATCAGTTTTTATAAACTTACACA | | | | |
| GlyTGA | GCCAGGGGTGTGGCCATACAGGTTTATAGTGG | Top 5′ (CysGCA-12- | 395.1 | 15.0 | 173 |
| combo3 | TTAGTAGAGACAAGTAAGTGGGTGCGTTGGTG | 1), top GlyTGA t- | | | |
| | GTATAGTGGTTAGCATAGCTGCCTtcaAAGCAGT | stem, 3′-35 bp (Tyr- | | | |
| | TGACCCCGGTTCGATTCCGGGCCAACGCAGTTC | GTA-6-1) | | | |
| | CATTTTTAAAAGTGTCTCTTCTGGGCCTGAA | | | | |
| GlyTGA | GCCAGGGGTGTGGCCATACAGGTTTATAGTGG | Top 5′ (CysGCA-12- | 369.0 | 3.6 | 174 |
| combo4 | TTAGTAGAGACAAGTAAGTGGGTGCGTTGGTG | 1), top GlyTGA t- | | | |
| | GTATAGTGGTTAGCATAGCTGCCTtcaAAGCAGT | stem, 3′-35 bp (Ile- | | | |
| | TGACCCCGGTTCGATTCCGGGCCAACGCAGACC | TAT-1-1, insert | | | |
| | CTTTTTTTCCCCATCAGTTTTTATAAACTTACAC | GACC) | | | |
| | A | | | | |
| GlyTGA | GCCAGGGGTGTGGCCATACAGGTTTATAGTGG | Top 5′ (CysGCA-12- | 295.5 | 9.4 | 175 |
| combo5 | TTAGTAGAGACAAGTAAGTGGGTGCGTTGGTG | 1), top GlyTGA t- | | | |
| | GTATAGTGGTTAGCATAGCTGCCTtcaAAGCAGT | stem, 3′-35 bp (Tyr- | | | |
| | TGACCCCGGTTCGATTCCGGGCCAACGCAGACC | GTA-6-1, insert | | | |
| | GTTCCATTTTTAAAAGTGTCTCTTCTGGGCCTGA | GACC) | | | |
| | A | | | | |
| GlyTGA | GCCAGGGGTGTGGCCATACAGGTTTATAGTGG | Top 5′ (CysGCA- | 458.3 | 12.7 | 176 |
| combo6 | TTAGTAGAGACAAGTAAGTGGGTGCGTTGGT | 12-1), top GlyTGA | | | |
| | GGTATAGTGGTTAGCATAGCTGCCTtcaAAGCA | t-stem, 3′-35 bp | | | |
| | GTTGACCCCGGTTCGATTCCGGGCCAACGCAG | (Ile-TAT-1-1, replace | | | |
| | ACCTTTTCCCCATCAGTTTTTATAAACTTACACA | GACC) | | | |
| GlyTGA | GCCAGGGGTGTGGCCATACAGGTTTATAGTGG | Top 5′ (CysGCA-12- | 356.1 | 2.1 | 177 |
| combo7 | TTAGTAGAGACAAGTAAGTGGGTGCGTTGGTG | 1), top GlyTGA t- | | | |
| | GTATAGTGGTTAGCATAGCTGCCTtcaAAGCAGT | stem, 3′-35 bp (Tyr- | | | |
| | TGACCCCGGTTCGATTCCGGGCCAACGCAGACC | GTA-6-1, replace | | | |
| | CATTTTTAAAAGTGTCTCTTCTGGGCCTGAA | GACC) | | | |
| 5pTyrGTA_ | AGCGCTCCGGTTTTTCTGTGCTGAACCTCAGGG | o5′ leader sequence, | 77.3 | 0.9 | 178 |
| 5_1LeuTGA | GACGCCGACACACGTACACGTCACCAGAATGGC | LeuTGA, o3′ trailer | | | |
| | CGAGTGGTtAAGGCGTTGGACTtcaGATCCAATG | sequence (serves as | | | |
| | GATTCATATCCGCGTGGGTTCGAACCCCACTTCT | LeuTGA reference | | | |
| | GGTAGTCCTTTTTTT | sample) | | | |
| 5pAlaTGC_ | TGTCCTGAGTTAAGCGACCAATAGGGACCATCT | Screen (Top 5) 5′ | 134.5 | 3.5 | 179 |
| 3_1LeuTGA | GGGAGAGCTGCTGAGAGTAGGTACCAGAATGG | leaders, LeuTGA, o3′ | | | |
| | CCGAGTGGTtAAGGCGTTGGACTtcaGATCCAAT | trailer sequence | | | |
| | GGATTCATATCCGCGTGGGTTCGAACCCCACTT | | | | |
| | CTGGTAGTCCTTTTTTT | | | | |
| 5pCysGCA_ | GCCAGGGGTGTGGCCATACAGGTTTATAGTGG | | 166.3 | 6.4 | 180 |
| 12_ | TTAGTAGAGACAAGTAAGTGGGTACCAGAATG | | | | |
| 1LeuTGA | GCCGAGTGGTtAAGGCGTTGGACTtcaGATCCA | | | | |
| | ATGGATTCATATCCGCGTGGGTTCGAACCCCAC | | | | |
| | TTCTGGTAGTCCTTTTTTT | | | | |
| 5pIleTAT_ | TCAATCTGTAAGAGCATCTTCTCTTATACCTTTC | | 119.1 | 2.5 | 181 |
| 3_1LeuTGA | CAAATCCTTATTATTTCTGTGACCAGAATGGCC | | | | |
| | GAGTGGTtAAGGCGTTGGACTtcaGATCCAATG | | | | |
| | GATTCATATCCGCGTGGGTTCGAACCCCACTTC | | | | |
| | TGGTAGTCCTTTTTTT | | | | |
| 5pLeuCAG_ | ATAGCGTTTAGACAAGAGACAGAAGTGAGTCC | | 198.9 | 63.5 | 182 |
| 1_7LeuTGA | TGAGCACAATGTAGCATAAGCGCACCAGAATG | | | | |
| | GCCGAGTGGTtAAGGCGTTGGACTtcaGATCCA | | | | |
| | ATGGATTCATATCCGCGTGGGTTCGAACCCCAC | | | | |
| | TTCTGGTAGTCCTTTTTTT | | | | |
| 5pTyrGTA_ | GATGAGGTTGCGGTAAGCATTAGAGGGCTATC | | 133.6 | 17.0 | 183 |
| 2_1LeuTGA | AGCAGCATCTTATCGCAGCGGAGACCAGAATG | | | | |
| | GCCGAGTGGTtAAGGCGTTGGACTtcaGATCCA | | | | |
| | ATGGATTCATATCCGCGTGGGTTCGAACCCCAC | | | | |
| | TTCTGGTAGTCCTTTTTTT | | | | |
| 3pGACC | AGCGCTCCGGTTTTTCTGTGCTGAACCTCAGGG | o5′ leader sequence, | 98.4 | 5.9 | 184 |
| LeuTGA | GACGCCGACACACGTACACGTCACCAGAATGG | LeuTGA, top 3′-4 bp | | | |
| | CCGAGTGGTtAAGGCGTTGGACTtcaGATCCAAT | (GACC) | | | |
| | GGATTCATATCCGCGTGGGTTCGAACCCCACTT | | | | |
| | CTGGTAGACCTTTTTTT | | | | |
| Top5p3p | GCCAGGGGTGTGGCCATACAGGTTTATAGTGG | Top 5′ (Cys-GCA-12- | 157.6 | 4.1 | 185 |
| LeuTGA | TTAGTAGAGACAAGTAAGTGGGTACCAGAATG | 1), LeuTGA, top 3′- | | | |
| | GCCGAGTGGTtAAGGCGTTGGACTtcaGATCCA | 4 bp (GACC) | | | |
| | ATGGATTCATATCCGCGTGGGTTCGAACCCCAC | | | | |
| | TTCTGGTAGACCTTTTTTT | | | | |
| LeuTGA | GCCAGGGGTGTGGCCATACAGGTTTATAGTGG | Top 5′ (CysGCA-12- | 158.0 | 1.9 | 186 |
| combo1 | TTAGTAGAGACAAGTAAGTGGGTACCAGAATG | 1), top LeuTGA t- | | | |
| | GCCGAGTGGTtAAGGCGTTGGACTtcaGATCCA | stem (ts), top 3′-4 | | | |
| | ATGGATTCATATCCGCCCGGGTTCGAACCCCGG | bp (GACC) | | | |
| | TTCTGGTAGACCTTTTTTT | | | | |
| LeuTGA | GCCAGGGGTGTGGCCATACAGGTTTATAGTGG | Top 5′ (CysGCA-12- | 814.5 | 44.6 | 187 |
| combo2 | TTAGTAGAGACAAGTAAGTGGGTGCCAGGATG | 1), top LeuTGA | | | |
| | GCCGAGTGGTTAAGGCGCCGGACTtcaGATCCG | sticky stem (SS), top | | | |
| | GTGGACTCATGTCCGCGTGGGTTCGAACCCCAC | 3′-4 bp (GACC) | | | |
| | TCCTGGCAGACCTTTTTTT | | | | |
| LeuTGA | GCCAGGGGTGTGGCCATACAGGTTTATAGTGG | Top 5′ (CysGCA-12- | 914.2 | 63.8 | 188 |
| combo3 | TTAGTAGAGACAAGTAAGTGGGTGCCAGGATG | 1), top LeuTGA t- | | | |
| | GCCGAGTGGTTAAGGCGCCGGACTtcaGATCCG | stem (ts), top 3′-4 | | | |
| | GTGGACTCATGTCCGCCCGGGTTCGAACCCCGG | bp (GACC) | | | |
| | TCCTGGCAGACCTTTTTTT | | | | |
| LeuTGA | GCCAGGGGTGTGGCCATACAGGTTTATAGTGG | Top 5′ (CysGCA-12- | 914.1 | 34.9 | 189 |
| combo4 | TTAGTAGAGACAAGTAAGTGGGTGCCAGGATG | 1), top LeuTGA ts | | | |
| | GCCGAGTGGTTAAGGCGCCGGACTtcaGATCCG | AND top SS, 3′- | | | |
| | GTGGACTCATGTCCGCCCGGGTTCGAACCCCGG | 35 bp (Ile-TAT-1-1) | | | |
| | TCCTGGCACTTTTTTTCCCCATCAGTTTTTATAAA | | | | |
| | CTTACACA | | | | |
| LeuTGA | GCCAGGGGTGTGGCCATACAGGTTTATAGTGG | Top 5′ (CysGCA-12- | 1012.2 | 6.5 | 190 |
| combo5 | TTAGTAGAGACAAGTAAGTGGGTGCCAGGATG | 1), top LeuTGA ts | | | |
| | GCCGAGTGGTTAAGGCGCCGGACTtcaGATCCG | AND top SS, 3′- | | | |
| | GTGGACTCATGTCCGCCCGGGTTCGAACCCCGG | 35 bp (Tyr-GTA-6-1) | | | |
| | TCCTGGCAGTTCCATTTTTAAAAGTGTCTCTTCT | | | | |
| | GGGCCTGAA | | | | |
| LeuTGA | GCCAGGGGTGTGGCCATACAGGTTTATAGTGG | Top 5′ (CysGCA-12- | 945.9 | 32.9 | 191 |
| combo6 | TTAGTAGAGACAAGTAAGTGGGTGCCAGGATG | 1), top LeuTGA ts | | | |
| | GCCGAGTGGTTAAGGCGCCGGACTtcaGATCCG | AND top SS, 3′- | | | |
| | GTGGACTCATGTCCGCCCGGGTTCGAACCCCGG | 35 bp (Ile-TAT-1-1, | | | |
| | TCCTGGCAGACCCTTTTTTTCCCCATCAGTTTTT | insert GACC) | | | |
| | ATAAACTTACACA | | | | |
| LeuTGA | GCCAGGGGTGTGGCCATACAGGTTTATAGTGG | Top 5′ (CysGCA-12- | 866.0 | 48.4 | 192 |
| combo7 | TTAGTAGAGACAAGTAAGTGGGTGCCAGGATG | 1), top LeuTGA ts | | | |
| | GCCGAGTGGTTAAGGCGCCGGACTtcaGATCCG | AND top SS, 3′- | | | |
| | GTGGACTCATGTCCGCCCGGGTTCGAACCCCGG | 35 bp (Tyr-GTA-6-1, | | | |
| | TCCTGGCAGACCGTTCCATTTTTAAAAGTGTCTC | insert GACC) | | | |
| | TTCTGGGCCTGAA | | | | |
| LeuTGA | GCCAGGGGTGTGGCCATACAGGTTTATAGTGG | Top 5′ (CysGCA- | 1118.5 | 85.8 | 193 |
| combo8 | TTAGTAGAGACAAGTAAGTGGGTGCCAGGAT | 12-1), top LeuTGA | | | |
| | GGCCGAGTGGTTAAGGCGCCGGACTtcaGATC | ts AND top SS, 3′- | | | |
| | CGGTGGACTCATGTCCGCCCGGGTTCGAACCC | 35 bp (Ile-TAT-1-1, | | | |
| | CGGTCCTGGCAGACCTTTTCCCCATCAGTTTTT | replace GACC) | | | |
| | ATAAACTTACACA | | | | |
| LeuTGA | GCCAGGGGTGTGGCCATACAGGTTTATAGTGG | Top 5′ (CysGCA-12- | 675.4 | 27.3 | 194 |
| combo9 | TTAGTAGAGACAAGTAAGTGGGTGCCAGGATG | 1), top LeuTGA ts | | | |
| | GCCGAGTGGTTAAGGCGCCGGACTtcaGATCCG | AND top SS, 3′- | | | |
| | GTGGACTCATGTCCGCCCGGGTTCGAACCCCGG | 35 bp (Tyr-GTA-6-1, | | | |
| | TCCTGGCAGACCCATTTTTAAAAGTGTCTCTTCT | replace GACC) | | | |
| | GGGCCTGAA | | | | |
| 5pTyrGTA_ | AGCGCTCCGGTTTTTCTGTGCTGAACCTCAGGG | o5′ leader sequence, | 20.1 | 1.3 | 195 |
| 5_1TrpTGA | GACGCCGACACACGTACACGTCGGCCTCGTGGC | LeuTGA, o3′ trailer | | | |
| | GCAACGGTAGCGCGTCTGACTtcaGATCAGAAG | sequence (serves as | | | |
| | GtTGCGTGTTCAAATCACGTCGGGGTCAGTCCTT | TrpTGA reference | | | |
| | TTTTT | sample) | | | |
| 5pAlaTGC_ | TGTCCTGAGTTAAGCGACCAATAGGGACCATCT | Screen (Top 5) 5′ | 27.4 | 1.2 | 196 |
| 3_1TrpTGA | GGGAGAGCTGCTGAGAGTAGGTGGCCTCGTGG | leaders, TrpTGA, o3′ | | | |
| | CGCAACGGTAGCGCGTCTGACTtcaGATCAGAA | trailer sequence | | | |
| | GGtTGCGTGTTCAAATCACGTCGGGGTCAGTCC | | | | |
| | TTTTTTT | | | | |
| 5pCysGCA_ | GCCAGGGGTGTGGCCATACAGGTTTATAGTGG | | 35.5 | 0.0 | 197 |
| 12_ | TTAGTAGAGACAAGTAAGTGGGTGGCCTCGTG | | | | |
| 1TrpTGA | GCGCAACGGTAGCGCGTCTGACTtcaGATCAGA | | | | |
| | AGGtTGCGTGTTCAAATCACGTCGGGGTCAGTC | | | | |
| | CTTTTTTT | | | | |
| 5pIleTAT_ | TCAATCTGTAAGAGCATCTTCTCTTATACCTTTC | | 32.9 | 1.2 | 198 |
| 3_1TrpTGA | CAAATCCTTATTATTTCTGTGGGCCTCGTGGCGC | | | | |
| | AACGGTAGCGCGTCTGACTtcaGATCAGAAGGt | | | | |
| | TGCGTGTTCAAATCACGTCGGGGTCAGTCCTTT | | | | |
| | TTTT | | | | |
| 5pLeuCAG_ | ATAGCGTTTAGACAAGAGACAGAAGTGAGTCC | | 31.7 | 3.7 | 199 |
| 1_7TrpTGA | TGAGCACAATGTAGCATAAGCGCGGCCTCGTG | | | | |
| | GCGCAACGGTAGCGCGTCTGACTtcaGATCAGA | | | | |
| | AGGtTGCGTGTTCAAATCACGTCGGGGTCAGTC | | | | |
| | CTTTTTTT | | | | |
| 5pTyrGTA_ | GATGAGGTTGCGGTAAGCATTAGAGGGCTATC | | 32.0 | 2.4 | 200 |
| 2_1TrpTGA | AGCAGCATCTTATCGCAGCGGAGGGCCTCGTG | | | | |
| | GCGCAACGGTAGCGCGTCTGACTtcaGATCAGA | | | | |
| | AGGtTGCGTGTTCAAATCACGTCGGGGTCAGTC | | | | |
| | CTTTTTTT | | | | |
| 3pGACC | AGCGCTCCGGTTTTTCTGTGCTGAACCTCAGGG | o5′ leader sequence, | 18.2 | 0.7 | 201 |
| TrpTGA | GACGCCGACACACGTACACGTCGGCCTCGTGG | TrpTGA, top 3′-4 bp | | | |
| | CGCAACGGTAGCGCGTCTGACTtcaGATCAGAA | (GACC) | | | |
| | GGtTGCGTGTTCAAATCACGTCGGGGTCAGACC | | | | |
| | TTTTTTT | | | | |
| Top5p3p | GCCAGGGGTGTGGCCATACAGGTTTATAGTGG | Top 5′ (Cys-GCA-12- | 35.6 | 0.7 | 202 |
| TrpTGA | TTAGTAGAGACAAGTAAGTGGGTGGCCTCGTG | 1), TrpTGA, top 3′- | | | |
| | GCGCAACGGTAGCGCGTCTGACTtcaGATCAGA | 4 bp (GACC) | | | |
| | AGGtTGCGTGTTCAAATCACGTCGGGGTCAGAC | | | | |
| | CTTTTTTT | | | | |
| TrpTGA | AGCGCTCCGGTTTTTCTGTGCTGAACCTCAGGG | o5′ leader sequence, | 69.3 | 5.9 | 203 |
| tsV10G52 | GACGCCGACACACGTACACGTCGGCCTCGTGG | top TrpTGA t-stem, | | | |
| C62 | CGCAACGGTAGCGCGTCTGACTtcaGATCAGAA | o3′ trailer sequence | | | |
| | GGtTCCGGGTTCAAATCCCGGCGGGGTCAGTCC | | | | |
| | TTTTTTT | | | | |
| TrpTGA | GCCAGGGGTGTGGCCATACAGGTTTATAGTGG | Top 5′ (CysGCA-12- | 95.2 | 5.2 | 204 |
| combo1 | TTAGTAGAGACAAGTAAGTGGGTGGCCTCGTG | 1), top TrpTGA t- | | | |
| | GCGCAACGGTAGCGCGTCTGACTtcaGATCAGA | stem, top 3′-4 bp | | | |
| | AGGtTCCGGGTTCAAATCCCGGCGGGGTCAGAC | (GACC) | | | |
| | CTTTTTTT | | | | |
| TrpTGA | GCCAGGGGTGTGGCCATACAGGTTTATAGTGG | (CysGCA-12-1), top | 88.5 | 5.8 | 205 |
| combo2 | TTAGTAGAGACAAGTAAGTGGGTGGCCTCGTG | TrpTGA t-stem, top | | | |
| | GCGCAACGGTAGCGCGTCTGACTtcaGATCAGA | 3′-35 bp (Ile-TAT-1- | | | |
| | AGGtTCCGGGTTCAAATCCCGGCGGGGTCACTT | 1) | | | |
| | TTTTTCCCCATCAGTTTTTATAAACTTACACA | | | | |
| TrpTGA | GCCAGGGGTGTGGCCATACAGGTTTATAGTGG | Top 5′ (CysGCA-12- | 85.3 | 1.7 | 206 |
| combo3 | TTAGTAGAGACAAGTAAGTGGGTGGCCTCGTG | 1), top TrpTGA t- | | | |
| | GCGCAACGGTAGCGCGTCTGACTtcaGATCAGA | stem, 3′-35 bp (Tyr- | | | |
| | AGGtTCCGGGTTCAAATCCCGGCGGGGTCAGTT | GTA-6-1) | | | |
| | CCATTTTTAAAAGTGTCTCTTCTGGGCCTGAA | | | | |
| TrpTGA | GCCAGGGGTGTGGCCATACAGGTTTATAGTGG | Top 5′ (CysGCA-12- | 87.9 | 8.7 | 207 |
| combo4 | TTAGTAGAGACAAGTAAGTGGGTGGCCTCGTG | 1), top TrpTGA t- | | | |
| | GCGCAACGGTAGCGCGTCTGACTtcaGATCAGA | stem, 3′-35 bp (Ile- | | | |
| | AGGtTCCGGGTTCAAATCCCGGCGGGGTCAGAC | TAT-1-1, insert | | | |
| | CCTTTTTTTCCCCATCAGTTTTTATAAACTTACAC | GACC) | | | |
| | A | | | | |
| TrpTGA | GCCAGGGGTGTGGCCATACAGGTTTATAGTGG | Top 5′ (CysGCA-12- | 82.7 | 1.0 | 208 |
| combo5 | TTAGTAGAGACAAGTAAGTGGGTGGCCTCGTG | 1), top TrpTGA t- | | | |
| | GCGCAACGGTAGCGCGTCTGACTtcaGATCAGA | stem, 3′-35 bp (Tyr- | | | |
| | AGGtTCCGGGTTCAAATCCCGGCGGGGTCAGAC | GTA-6-1, insert | | | |
| | CGTTCCATTTTTAAAAGTGTCTCTTCTGGGCCTG | GACC) | | | |
| | AA | | | | |
| TrpTGA | GCCAGGGGTGTGGCCATACAGGTTTATAGTGG | Top 5′ (CysGCA- | 93.1 | 12.0 | 209 |
| combo6 | TTAGTAGAGACAAGTAAGTGGGTGGCCTCGTG | 12-1), top TrpTGA | | | |
| | GCGCAACGGTAGCGCGTCTGACTtcaGATCAGA | t-stem, 3′-35 bp | | | |
| | AGGtTCCGGGTTCAAATCCCGGCGGGGTCAGA | (Ile-TAT-1-1, replace | | | |
| | CCTTTTCCCCATCAGTTTTTATAAACTTACACA | GACC) | | | |
| TrpTGA | GCCAGGGGTGTGGCCATACAGGTTTATAGTGG | Top 5′ (CysGCA-12- | 76.0 | 6.9 | 210 |
| combo7 | TTAGTAGAGACAAGTAAGTGGGTGGCCTCGTG | 1), top TrpTGA t- | | | |
| | GCGCAACGGTAGCGCGTCTGACTtcaGATCAGA | stem, 3′-35 bp (Tyr- | | | |
| | AGGtTCCGGGTTCAAATCCCGGCGGGGTCAGAC | GTA-6-1, replace | | | |
| | CCATTTTTAAAAGTGTCTCTTCTGGGCCTGAA | GACC) | | | |

Key:
1. Italic: Original ACE-tRNA Sequence
2. Bold:. Top Fully Optimized ACE-tRNA Sequence

Additional optimized and original ACE-tRNA sequences and data are also shown in Table 10 below. In this table, those in rows 2-18 are the sequences from Lueck et al., Nat Commun 2019, 10 (1), 822. They are the top sequences for each ACE-tRNA family (isoacceptor/stop codon) flanked with the original 5′ sequence and 3′ sequence from that study. These serve as the reference/parent sequences for the following sequences.

The sequences with the “5p3p” prefix (rows 19-30) use the ACE-tRNA sequences as above but swap the 5′ sequence and 3′ sequence to the best ones discovered and disclosed herein. These correspond to the 5′ and 3′ sequences used for those bold sequences in Table 9 “Best Combos.” But these sequences are distinct from those listed in Table 9 “Best Combos” because nothing within the ACE-tRNA sequence is changed for those listed in rows 19-30, just the 5′ and 3′ sequences.

The sequences with the “TV10” prefix (rows 31-39) have the same 5′ and 3′ sequences as the “parent” sequences but have the t-stem within the ACE-tRNA sequence altered. Because t-stem version 10 worked better for several of the Arg, Gly, Leu sequences it was decided to exchange the native t-stem in all of the ACE-tRNAs for this sequence.

Because the Ser ACE-tRNAs were improved by both the 5p3p and TV10, and because the Ser ACE-tRNAs have especially high activity and are possibly amenable to many mutations, the 5p3p with the TV10 together were also examined.

TABLE 10
All tRNAs 5p3pt

| | | Average | | |
|---|---|---|---|---|
| | | Suppression | | |
| | | Ratio | | |
| | | Normalized | | SEQ |
| | | to Parent | | ID |
| Name | ACE-tRNA Expression Cassette Sequence | ACE-tRNA | S.E.M. | NO. |
| tRNA_Gly_TCC_ | AGCGCTCCGGTTTTTCTGTGCTGAACCTCAGGGGACGCCGAC | 1.00 | 0.04 | 211 |
| 1_1TGA | ACACGTACACGTCGCGTTGGTGGTATAGTGGTtAGCATAGCT | | | |
| | GCCTtcaAAGCAGTTGaCCCGGGTTCGATTCCCGGCCAACGCA | | | |
| | GTCCTTTTTTT | | | |
| tRNA_Trp_CCA_ | AGCGCTCCGGTTTTTCTGTGCTGAACCTCAGGGGACGCCGAC | 1.00 | 0.01 | 212 |
| 1_1TGA | ACACGTACACGTCGGCCTCGTGGCGCAACGGTAGCGCGTCT | | | |
| | GACTtcaGATCAGAAGGtTGCGTGTTCAAATCACGTCGGGGTC | | | |
| | AGTCCTTTTTTT | | | |
| tRNA_Trp_CCA_ | AGCGCTCCGGTTTTTCTGTGCTGAACCTCAGGGGACGCCGAC | 1.00 | 0.01 | 213 |
| 2_1TAG | ACACGTACACGTCGACCTCGTGGCGCAATGGTAGCGCGTCTG | | | |
| | ACTctaGATCAGAAGGtTGCGTGTTCAAGTCACGTCGGGGTCA | | | |
| | GTCCTTTTTTT | | | |
| tRNA_Arg_TCT_ | AGCGCTCCGGTTTTTCTGTGCTGAACCTCAGGGGACGCCGAC | 1.00 | 0.03 | 214 |
| 3_1TGA | ACACGTACACGTCGGCTCTGTGGCGCAATGGAtAGCGCATTG | | | |
| | GACTtcaAATTCAAAGGtTGTGGGTTCGAGTCCCACCAGAGTC | | | |
| | GGTCCTTTTTTT | | | |
| tRNA_Gln_CTG_ | AGCGCTCCGGTTTTTCTGTGCTGAACCTCAGGGGACGCCGAC | 1.00 | 0.05 | 215 |
| 3_1TAG | ACACGTACACGTCGGTTCCATGGTGTAATGGTgAGCACTCTG | | | |
| | GACTctaAATCCAGCGaTCCGAGTTCGAGTCTCGGTGGAACCT | | | |
| | GTCCTTTTTTT | | | |
| tRNA_Gln_CTG_ | AGCGCTCCGGTTTTTCTGTGCTGAACCTCAGGGGACGCCGAC | 1.00 | 0.01 | 216 |
| 4_1TAA | ACACGTACACGTCGGTTCCATGGTGTAATGGTaAGCACTCTG | | | |
| | GACTttaAATCCAGCGaTCCGAGTTCGAGTCTCGGTGGAACCT | | | |
| | GTCCTTTTTTT | | | |
| tRNA_Glu_TTC_ | AGCGCTCCGGTTTTTCTGTGCTGAACCTCAGGGGACGCCGAC | 1.00 | 0.08 | 217 |
| 2_1TAG | ACACGTACACGTCTCCCACATGGTCTAGCGGTtAGGATTCCTG | | | |
| | GTTctaACCCAGGCGGCCCGGGTTCGACTCCCGGTGTGGGAA | | | |
| | GTCCTTTTTTT | | | |
| tRNA_Glu_TTC_ | AGCGCTCCGGTTTTTCTGTGCTGAACCTCAGGGGACGCCGAC | 1.00 | 0.03 | 218 |
| 2_1TAA | ACACGTACACGTCTCCCACATGGTCTAGCGGTtAGGATTCCTG | | | |
| | GTTttaACCCAGGCGGCCCGGGTTCGACTCCCGGTGTGGGAA | | | |
| | GTCCTTTTTTT | | | |
| tRNA_Tyr_GTA_ | AGCGCTCCGGTTTTTCTGTGCTGAACCTCAGGGGACGCCGAC | 1.00 | 0.03 | 219 |
| 5_1TAA | ACACGTACACGTCCCTTCGATAGCTCAGCTGGTAGAGCGGAG | | | |
| | GACTttaGATCCTTAGGtCGCTGGTTCGATTCCGGCTCGAAGG | | | |
| | AGTCCTTTTTTT | | | |
| tRNA_Tyr_GTA_ | AGCGCTCCGGTTTTTCTGTGCTGAACCTCAGGGGACGCCGAC | 1.00 | 0.02 | 220 |
| 5_1TAG | ACACGTACACGTCCCTTCGATAGCTCAGCTGGTAGAGCGGAG | | | |
| | GACTctaGATCCTTAGGtCGCTGGTTCGATTCCGGCTCGAAGG | | | |
| | AGTCCTTTTTTT | | | |
| tRNA_Leu_AAG_ | AGCGCTCCGGTTTTTCTGTGCTGAACCTCAGGGGACGCCGAC | 1.00 | 0.04 | 221 |
| 3_1TAG | ACACGTACACGTCGGTAGCGTGGCCGAGCGGTctAAGGCGCT | | | |
| | GGATTctaGCTCCAGTCTCTTCGGGGGCGTGGGTTCAAATCCC | | | |
| | ACCGCTGCCAGTCCTTTTTTT | | | |
| tRNA_Leu_TAA_ | AGCGCTCCGGTTTTTCTGTGCTGAACCTCAGGGGACGCCGAC | 1.00 | 0.02 | 222 |
| 3_1TGA | ACACGTACACGTCACCAGAATGGCCGAGTGGTtAAGGCGTTG | | | |
| | GACTtcaGATCCAATGGATTCATATCCGCGTGGGTTCGAACCC | | | |
| | CACTTCTGGTAGTCCTTTTTTT | | | |
| tRNA_Ser_GCT_ | AGCGCTCCGGTTTTTCTGTGCTGAACCTCAGGGGACGCCGAC | 1.00 | 0.05 | 223 |
| 5_1TAG | ACACGTACACGTCGACGAGGTGGCCGAGTGGTtAAGGCGAT | | | |
| | GGACTctaAATCCATTGTGCTCTGCACACGTGGGTTCGAATCC | | | |
| | CATCCTCGTCGGTCCTTTTTTT | | | |
| tRNA_Ser_CGA_ | AGCGCTCCGGTTTTTCTGTGCTGAACCTCAGGGGACGCCGAC | 1.00 | 0.03 | 224 |
| 4_1TGA | ACACGTACACGTCGTCACGGTGGCCGAGTGGTtAAGGCGTTG | | | |
| | GACTtcaAATCCAATGGGGTTTCCCCGCACAGGTTCGAATCCT | | | |
| | GTTCGTGACGGTCCTTTTTTT | | | |
| tRNA_Ser_AGA_ | AGCGCTCCGGTTTTTCTGTGCTGAACCTCAGGGGACGCCGAC | 1.00 | 0.03 | 225 |
| 3_1TAA | ACACGTACACGTCGTAGTCGTGGCCGAGTGGTtAAGGCGAT | | | |
| | GGACTttaAATCCATTGGGGTTTCCCCACGCAGGTTCGAATCC | | | |
| | TGCCGACTACGGTCCTTTTTTT | | | |
| tRNA_Lys_TTT_ | AGCGCTCCGGTTTTTCTGTGCTGAACCTCAGGGGACGCCGAC | 1.00 | 0.05 | 226 |
| 6_1TAA | ACACGTACACGTCGCCTGGGTAGCTCAGTCGGTAGAGCATCA | | | |
| | GACTttaAATCTGAGGGtCCAGGGTTCAAGTCCCTGTCCAGGC | | | |
| | GGTCCTTTTTTT | | | |
| tRNA_Cys_GCA_ | AGCGCTCCGGTTTTTCTGTGCTGAACCTCAGGGGACGCCGAC | 1.00 | 0.14 | 227 |
| 5_1TGA | ACACGTACACGTCGGGGGTATAGCTCAGTGGGTAGAGCATT | | | |
| | TGACTtcaGATCAAGAGGtCCCCGGTTCAAATCCGGGTGCCCC | | | |
| | CTGTCCTTTTTTT | | | |
| 5p3p_tRNA_Gly_ | GCCAGGGGTGTGGCCATACAGGTTTATAGTGGTTAGTAGAG | 2.12 | 0.04 | 228 |
| TCC_1_1TGA | ACAAGTAAGTGGGTGCGTTGGTGGTATAGTGGTtAGCATAG | | | |
| | CTGCCTtcaAAGCAGTTGaCCCGGGTTCGATTCCCGGCCAACG | | | |
| | CAGACCTTTTCCCCATCAGTTTTTATAAACTTACACA | | | |
| 5p3p_tRNA_Trp_ | GCCAGGGGTGTGGCCATACAGGTTTATAGTGGTTAGTAGAG | 2.14 | 0.05 | 229 |
| CCA_1_1TGA | ACAAGTAAGTGGGTGGCCTCGTGGCGCAACGGTAGCGCGTC | | | |
| | TGACTtcaGATCAGAAGGtTGCGTGTTCAAATCACGTCGGGGT | | | |
| | CAGACCTTTTCCCCATCAGTTTTTATAAACTTACACA | | | |
| 5p3p_tRNA_Trp_ | GCCAGGGGTGTGGCCATACAGGTTTATAGTGGTTAGTAGAG | 1.87 | 0.10 | 230 |
| CCA_2_1TAG | ACAAGTAAGTGGGTGACCTCGTGGCGCAATGGTAGCGCGTC | | | |
| | TGACTctaGATCAGAAGGtTGCGTGTTCAAGTCACGTCGGGGT | | | |
| | CAGACCTTTTCCCCATCAGTTTTTATAAACTTACACA | | | |
| 5p3p_tRNA_Arg_ | GCCAGGGGTGTGGCCATACAGGTTTATAGTGGTTAGTAGAG | 1.74 | 0.04 | 231 |
| TCT_3_1TGA | ACAAGTAAGTGGGTGGCTCTGTGGCGCAATGGAtAGCGCAT | | | |
| | TGGACTtcaAATTCAAAGGtTGTGGGTTCGAGTCCCACCAGAG | | | |
| | TCGGACCTTTTCCCCATCAGTTTTTATAAACTTACACA | | | |
| 5p3p_tRNA_Gln_ | GCCAGGGGTGTGGCCATACAGGTTTATAGTGGTTAGTAGAG | 1.99 | 0.04 | 232 |
| CTG_3_1TAG | ACAAGTAAGTGGGTGGTTCCATGGTGTAATGGTgAGCACTCT | | | |
| | GGACTctaAATCCAGCGaTCCGAGTTCGAGTCTCGGTGGAAC | | | |
| | CTGACCTTTTCCCCATCAGTTTTTATAAACTTACACA | | | |
| 5p3p_tRNA_Gln_ | GCCAGGGGTGTGGCCATACAGGTTTATAGTGGTTAGTAGAG | 1.34 | 0.04 | 233 |
| CTG_4_1TAA | ACAAGTAAGTGGGTGGTTCCATGGTGTAATGGTaAGCACTCT | | | |
| | GGACTttaAATCCAGCGaTCCGAGTTCGAGTCTCGGTGGAACC | | | |
| | TGACCTTTTCCCCATCAGTTTTTATAAACTTACACA | | | |
| 5p3p_tRNA_Leu_ | GCCAGGGGTGTGGCCATACAGGTTTATAGTGGTTAGTAGAG | 1.49 | 0.08 | 234 |
| AAG_3_1TAG | ACAAGTAAGTGGGTGGTAGCGTGGCCGAGCGGTctAAGGCG | | | |
| | CTGGATTctaGCTCCAGTCTCTTCGGGGGCGTGGGTTCAAATC | | | |
| | CCACCGCTGCCAGACCTTTTCCCCATCAGTTTTTATAAACTTAC | | | |
| | ACA | | | |
| 5p3p_tRNA_Leu_ | GCCAGGGGTGTGGCCATACAGGTTTATAGTGGTTAGTAGAG | 1.18 | 0.06 | 235 |
| TAA_3_1TGA | ACAAGTAAGTGGGTACCAGAATGGCCGAGTGGTtAAGGCGT | | | |
| | TGGACTtcaGATCCAATGGATTCATATCCGCGTGGGTTCGAAC | | | |
| | CCCACTTCTGGTAGACCTTTTCCCCATCAGTTTTTATAAACTTA | | | |
| | CACA | | | |
| 5p3p_tRNA_Ser_ | GCCAGGGGTGTGGCCATACAGGTTTATAGTGGTTAGTAGAG | 1.58 | 0.06 | 236 |
| GCT_5_1TAG | ACAAGTAAGTGGGTGACGAGGTGGCCGAGTGGTtAAGGCGA | | | |
| | TGGACTctaAATCCATTGTGCTCTGCACACGTGGGTTCGAATC | | | |
| | CCATCCTCGTCGGACCTTTTCCCCATCAGTTTTTATAAACTTAC | | | |
| | ACA | | | |
| 5p3p_tRNA_Ser_ | GCCAGGGGTGTGGCCATACAGGTTTATAGTGGTTAGTAGAG | 1.91 | 0.06 | 237 |
| CGA_4_1TGA | ACAAGTAAGTGGGTGTCACGGTGGCCGAGTGGTtAAGGCGT | | | |
| | TGGACTtcaAATCCAATGGGGTTTCCCCGCACAGGTTCGAATC | | | |
| | CTGTTCGTGACGGACCTTTTCCCCATCAGTTTTTATAAACTTA | | | |
| | CACA | | | |
| 5p3p_tRNA_Ser_ | GCCAGGGGTGTGGCCATACAGGTTTATAGTGGTTAGTAGAG | 2.58 | 0.14 | 238 |
| AGA_3_1TAA | ACAAGTAAGTGGGTGTAGTCGTGGCCGAGTGGTtAAGGCGA | | | |
| | TGGACTttaAATCCATTGGGGTTTCCCCACGCAGGTTCGAATC | | | |
| | CTGCCGACTACGGACCTTTTCCCCATCAGTTTTTATAAACTTA | | | |
| | CACA | | | |
| 5p3p_tRNA_Lys_ | GCCAGGGGTGTGGCCATACAGGTTTATAGTGGTTAGTAGAG | 3.14 | 0.19 | 239 |
| TTT_6_1TAA | ACAAGTAAGTGGGTGCCTGGGTAGCTCAGTCGGTAGAGCAT | | | |
| | CAGACTttaAATCTGAGGGtCCAGGGTTCAAGTCCCTGTCCAG | | | |
| | GCGGACCTTTTCCCCATCAGTTTTTATAAACTTACACA | | | |
| TV10_tRNA_Trp_ | AGCGCTCCGGTTTTTCTGTGCTGAACCTCAGGGGACGCCGAC | 2.06 | 0.05 | 240 |
| CCA_1_1TGA | ACACGTACACGTCGGCCTCGTGGCGCAACGGTAGCGCGTCT | | | |
| | GACTtcaGATCAGAAGGtTccgggTTCAAATcccggCGGGGTCAG | | | |
| | TCCTTTTTTT | | | |
| TV10_tRNA_Glu_ | AGCGCTCCGGTTTTTCTGTGCTGAACCTCAGGGGACGCCGAC | 1.67 | 0.05 | 241 |
| TTC_2_1TAG | ACACGTACACGTCTCCCACATGGTCTAGCGGTtAGGATTCCTG | | | |
| | GTTctaACCCAGGCGGCccgggTTCGACTcccggTGTGGGAAGT | | | |
| | CCTTTTTTT | | | |
| TV10_tRNA_Glu_ | AGCGCTCCGGTTTTTCTGTGCTGAACCTCAGGGGACGCCGAC | 1.33 | 0.02 | 242 |
| TTC_2_1TAA | ACACGTACACGTCTCCCACATGGTCTAGCGGTtAGGATTCCTG | | | |
| | GTTttaACCCAGGCGGCccgggTTCGACTcccggTGTGGGAAGTC | | | |
| | CTTTTTTT | | | |
| TV10_tRNA_Tyr_ | AGCGCTCCGGTTTTTCTGTGCTGAACCTCAGGGGACGCCGAC | 1.30 | 0.03 | 243 |
| GTA_5_1TAA | ACACGTACACGTCCCTTCGATAGCTCAGCTGGTAGAGCGGAG | | | |
| | GACTttaGATCCTTAGGtCccgggTTCGATTcccggTCGAAGGAG | | | |
| | TCCTTTTTTT | | | |
| TV10_tRNA_Tyr_ | AGCGCTCCGGTTTTTCTGTGCTGAACCTCAGGGGACGCCGAC | 1.10 | 0.02 | 244 |
| GTA_5_1TAG | ACACGTACACGTCCCTTCGATAGCTCAGCTGGTAGAGCGGAG | | | |
| | GACTctaGATCCTTAGGtCccgggTTCGATTcccggTCGAAGGAG | | | |
| | TCCTTTTTTT | | | |
| TV10_tRNA_Ser_ | AGCGCTCCGGTTTTTCTGTGCTGAACCTCAGGGGACGCCGAC | 1.37 | 0.03 | 245 |
| GCT_5_1TAG | ACACGTACACGTCGACGAGGTGGCCGAGTGGTtAAGGCGAT | | | |
| | GGACTctaAATCCATTGTGCTCTGCACACccgggTTCGAATcccg | | | |
| | gCCTCGTCGGTCCTTTTTTT | | | |
| TV10_tRNA_Ser_ | AGCGCTCCGGTTTTTCTGTGCTGAACCTCAGGGGACGCCGAC | 1.68 | 0.04 | 246 |
| CGA_4_1TGA | ACACGTACACGTCGTCACGGTGGCCGAGTGGTtAAGGCGTTG | | | |
| | GACTtcaAATCCAATGGGGTTTCCCCGCccgggTTCGAATcccgg | | | |
| | TCGTGACGGTCCTTTTTTT | | | |
| TV10_tRNA_Ser_ | AGCGCTCCGGTTTTTCTGTGCTGAACCTCAGGGGACGCCGAC | 1.48 | 0.05 | 247 |
| AGA_3_1TAA | ACACGTACACGTCGTAGTCGTGGCCGAGTGGTtAAGGCGAT | | | |
| | GGACTttaAATCCATTGGGGTTTCCCCACccgggTTCGAATcccg | | | |
| | gCGACTACGGTCCTTTTTTT | | | |
| TV10_tRNA_Cys_ | AGCGCTCCGGTTTTTCTGTGCTGAACCTCAGGGGACGCCGAC | 1.89 | 0.07 | 248 |
| GCA_5_1TGA | ACACGTACACGTCGGGGGTATAGCTCAGTGGGTAGAGCATT | | | |
| | TGACTtcaGATCAAGAGGtCccgggTTCAAATcccggTGCCCCCT | | | |
| | GTCCTTTTTTT | | | |
| 5p3p_TV10_ | GCCAGGGGTGTGGCCATACAGGTTTATAGTGGTTAGTAGAG | 1.44 | 0.12 | 249 |
| tRNA_Ser_GCT_ | ACAAGTAAGTGGGTGACGAGGTGGCCGAGTGGTtAAGGCGA | | | |
| 5_1TAG | TGGACTctaAATCCATTGTGCTCTGCACACccgggTTCGAATccc | | | |
| | ggCCTCGTCGGACCTTTTCCCCATCAGTTTTTATAAACTTACAC | | | |
| | A | | | |
| 5p3p_TV10_ | GCCAGGGGTGTGGCCATACAGGTTTATAGTGGTTAGTAGAG | 1.90 | 0.06 | 250 |
| tRNA_Ser_CGA_ | ACAAGTAAGTGGGTGTCACGGTGGCCGAGTGGTtAAGGCGT | | | |
| 4_1TGA | TGGACTtcaAATCCAATGGGGTTTCCCCGCccgggTTCGAATccc | | | |
| | ggTCGTGACGGACCTTTTCCCCATCAGTTTTTATAAACTTACAC | | | |
| | A | | | |
| 5p3p_TV10_ | GCCAGGGGTGTGGCCATACAGGTTTATAGTGGTTAGTAGAG | 2.64 | 0.05 | 251 |
| tRNA_Ser_AGA_ | ACAAGTAAGTGGGTGTAGTCGTGGCCGAGTGGTtAAGGCGA | | | |
| 3_1TAA | TGGACTttaAATCCATTGGGGTTTCCCCACccgggTTCGAATccc | | | |
| | ggCGACTACGGACCTTTTCCCCATCAGTTTTTATAAACTTACAC | | | |
| | A | | | |

### Example 11

In this example, assays were carried out to show that optimized ACE-tRNAs retained their fidelity in translation.

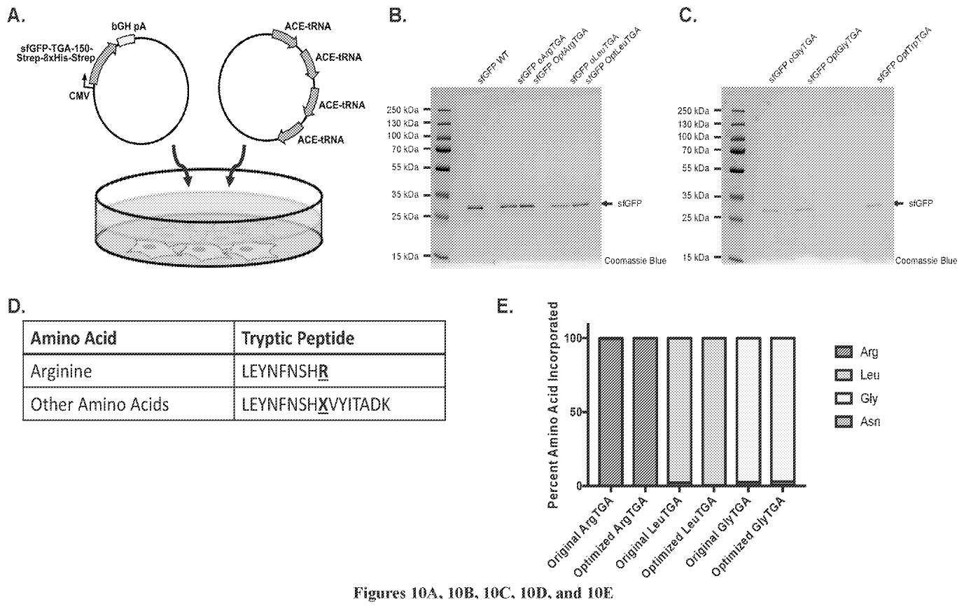

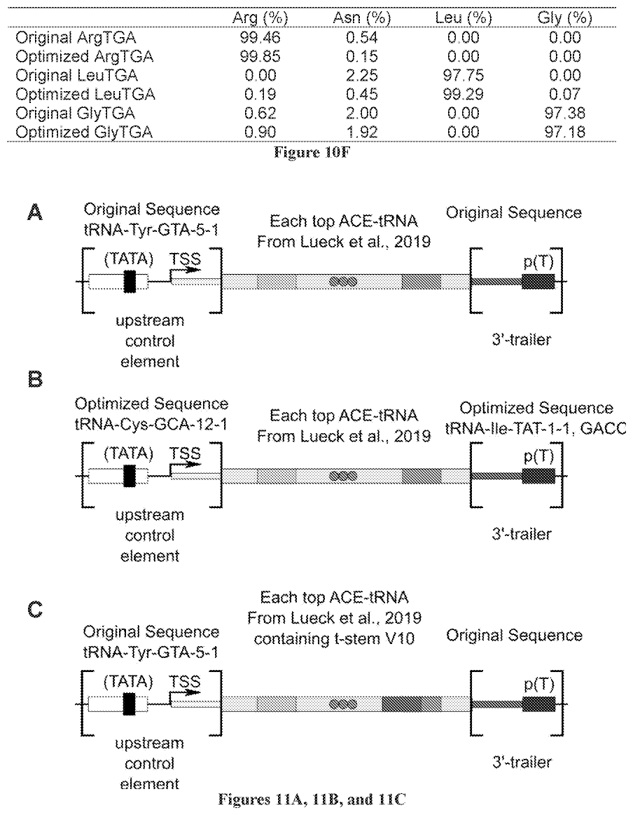

A pcDNA3.1 (+) plasmid encoding superfolder green fluorescent protein (sfGFP) with a TGA stop codon at amino acid position 150 and a C-terminal Strep-8xHistidine-Strep tag was co-transfected with a plasmid encoding 4 copies of the ACE-tRNA under investigation into HEK293T cells (FIG. 10A). Two days after transfection, the cells were harvested and thoroughly lysed with a dounce homogenizer in PBS supplemented with protease inhibitor cocktail. Full-length sfGFP was purified via the C-terminal Strep-8xHis-Strep tag using Strep-Tactin XT Superflow resin (IBA Lifesciences) as per manufacturer instructions. The eluted sfGFP protein was resolved on a 10-20% gradient SDS-PAGE gel and stained with SimplyBlue Safestain (Thermo Fisher Scientific) Coomassie stain (FIG. 10B-C). The sfGFP was excised from the gel and subjected to trypsin digest followed by mass spectrometric determination of the resulting peptide masses. The mass of each peptide which contained the amino acid at site 150 in sfGFP (FIG. 10D) was determined and the amino acid with the most consistent mass for position 150 in that peptide was determined. As trypsin cleaves after arginine and lysine, the peptide for arginine at position 150 is different than for the other amino acids shown here (arginine in the peptide denoted as R, other amino acids denoted as X). For each protein purified, the amount (peptide abundance) of each amino acid incorporated at position 150 was tallied and expressed as a percent (FIG. 10E).

For all ACE-tRNAs under consideration here, >97% of the amino acid incorporated at the PTC (site 150) was the cognate amino acid (i.e. the amino acid specified for by the identity of the parent tRNA from which the ACE-tRNA was derived). These results indicate that translational fidelity was largely retained following optimization of the ACE-tRNA sequences.

### REFERENCES

1 Lueck, J. D.; Yoon, J. S.; Perales-Puchalt, A.; Mackey, A. L.; Infield, D. T.; Behlke, M. A.; Pope, M. R.; Weiner, D. B.; Skach, W. R.; McCray, P. B., Jr.; Ahern, C. A., Engineered transfer RNAs for suppression of premature termination codons. Nat Commun 2019, 10 (1), 822.
2. Chatterjee, A.; Sun, S. B.; Furman, J. L.; Xiao, H.; Schultz, P. G., A versatile platform for single- and multiple-unnatural amino acid mutagenesis in Escherichia coli. Biochemistry 2013, 52 (10), 1828-37.
3. Serfling, R.; Lorenz, C.; Etzel, M.; Schicht, G.; Bottke, T.; Morl, M.; Coin, I., Designer tRNAs for efficient incorporation of non-canonical amino acids by the pyrrolysine system in mammalian cells. Nucleic Acids Res 2018, 46 (1), 1-10.
4. Guo, J.; Melancon, C. E., 3rd; Lee, H. S.; Groff, D.; Schultz, P. G., Evolution of amber suppressor tRNAs for efficient bacterial production of proteins containing nonnatural amino acids. Angew Chem Int Ed Engl 2009,48 (48), 9148-51.
5. Rauch, B. J.; Porter, J. J.; Mehl, R. A.; Perona, J. J., Improved Incorporation of Noncanonical Amino Acids by an Engineered tRNA (Tyr) Suppressor. Biochemistry 2016, 55 (3), 618-28.
6. Keeling, K. M.; Xue, X.; Gunn, G.; Bedwell, D. M., Therapeutics based on stop codon readthrough. AnnuRev Genomics Hum Genet 2014, 15, 371-94.
7. Rowe, S. M.; Miller, S.; Sorscher, E. J., Cystic fibrosis. N Engl J Med 2005, 352 (19), 1992-2001.
8. Shalev, M.; Baasov, T., When Proteins Start to Make Sense: Fine-tuning Aminoglycosides for PTC Suppression Therapy. Medchemcomm 2014, 5 (8), 1092-1105.
9. Baradaran-Heravi, A.; Balgi, A. D.; Zimmerman, C.; Choi, K.; Shidmoossavee, F. S.; Tan, J. S.; Bergeaud, C.; Krause, A.; Flibotte, S.; Shimizu, Y.; Anderson, H. J.; Mouly, V.; Jan, E.; Pfeifer, T.; Jaquith, J. B.; Roberge, M., Novel small molecules potentiate premature termination codon readthrough by aminoglycosides. Nucleic Acids Res 2016, 44 (14), 6583-98.
10. Schacht, J., Antioxidant therapy attenuates aminoglycoside-induced hearing loss. Ann N Y Acad Sci 1999, 884, 125-30.
11. Sabbavarapu, N. M.; Shavit, M.; Degani, Y.; Smolkin, B.; Belakhov, V.; Baasov, T., Design of Novel Aminoglycoside Derivatives with Enhanced Suppression of Diseases-Causing Nonsense Mutations. ACS Med Chem Lett 2016, 7 (4), 418-23.
12. Bedwell, D. M.; Kaenjak, A.; Benos, D. J.; Bebok, Z.; Bubien, J. K.; Hong, J.; Tousson, A.; Clancy, J. P.; Sorscher, E. J., Suppression of a CFTR premature stop mutation in a bronchial epithelial cell line. Nat Med 1997,3 (11), 1280-4.
13. Mutyam, V.; Du, M.; Xue, X.; Keeling, K. M.; White, E. L.; Bostwick, J. R.; Rasmussen, L.; Liu, B.; Mazur, M.; Hong, J. S.; Falk Libby, E.; Liang, F.; Shang, H.; Mense, M.; Suto, M. J.; Bedwell, D. M.; Rowe, S. M., Discovery of Clinically Approved Agents That Promote Suppression of Cystic Fibrosis Transmembrane Conductance Regulator Nonsense Mutations. Am J Respir Crit Care Med 2016, 194 (9), 1092-1103.
14. Xue, X.; Mutyam, V.; Tang, L.; Biswas, S.; Du, M.; Jackson, L. A.; Dai, Y.; Belakhov, V.; Shalev, M.; Chen, F.; Schacht, J.; R, J. B.; Baasov, T.; Hong, J.; Bedwell, D. M.; Rowe, S. M., Synthetic aminoglycosides efficiently suppress cystic fibrosis transmembrane conductance regulator nonsense mutations and are enhanced by ivacaftor. Am J Respir Cell Mol Biol 2014, 50 (4), 805-16.
15. Zomer-van Ommen, D. D.; Vijftigschild, L. A.; Kruisselbrink, E.; Vonk, A. M.; Dekkers, J. F.; Janssens, H. M.; de Winter-de Groot, K. M.; van der Ent, C. K.; Beekman, J. M., Limited premature termination codon suppression by read-through agents in cystic fibrosis intestinal organoids. J Cyst Fibros 2016, 15 (2), 158-62.
16. Leubitz, A.; Frydman-Marom, A.; Sharpe, N.; van Duzer, J.; Campbell, K. C. M.; Vanhoutte, F., Safety, Tolerability, and Pharmacokinetics of Single Ascending Doses of ELX-02, a Potential Treatment for Genetic Disorders Caused by Nonsense Mutations, in Healthy Volunteers. Clin Pharmacol Drug Dev 2019, 8 (8), 984-994.
17. Temple, G. F.; Dozy, A. M.; Roy, K. L.; Kan, Y. W., Construction of a functional human suppressor tRNAgene: an approach to gene therapy for beta-thalassaemia. Nature 1982, 296 (5857), 537-40.
18. Kiselev, A. V.; Ostapenko, O. V.; Rogozhkina, E. V.; Kholod, N. S.; Seit Nebi, A. S.; Baranov, A. N.; Lesina, E. A.; Ivashchenko, T. E.; Sabetskii, V. A.; Shavlovskii, M. M.; Rechinskii, V. O.; Kiselev, L. L.; Baranov, V. C., [Suppression of nonsense mutations in the Dystrophin gene by a suppressor tRNA gene]. Mol Biol (Mosk) 2002, 36 (1), 43-7.
19. Chin, J. W., Expanding and reprogramming the genetic code of cells and animals. Annu Rev Biochem 2014, 83, 379-408.
20. Elliott, T. S.; Bianco, A.; Chin, J. W., Genetic code expansion and bioorthogonal labelling enables cell specific proteomics in an animal. Curr Opin Chem Biol 2014, 21, 154-60.
21. Greiss, S.; Chin, J. W., Expanding the genetic code of an animal. J Am Chem Soc 2011, 133 (36), 14196-9.
22. Liu, J.; Hemphill, J.; Samanta, S.; Tsang, M.; Deiters, A., Genetic Code Expansion in Zebrafish Embryosand Its Application to Optical Control of Cell Signaling. J Am Chem Soc 2017, 139 (27), 9100-9103.
23. Xue, X.; Mutyam, V.; Thakerar, A.; Mobley, J.; Bridges, R. J.; Rowe, S. M.; Keeling, K. M.; Bedwell, D. M., Identification of the amino acids inserted during suppression of CFTR nonsense mutations and determination of their functional consequences. Hum Mol Genet 2017, 26 (16), 3116-3129.
24. Ye, S.; Kohrer, C.; Huber, T.; Kazmi, M.; Sachdev, P.; Yan, E. C.; Bhagat, A.; RajBhandary, U. L.; Sakmar, T. P., Site-specific incorporation of keto amino acids into functional G protein-coupled receptors using unnatural amino acid mutagenesis. J Biol Chem 2008, 283 (3), 1525-33.
25. Mukai, T.; Kobayashi, T.; Hino, N.; Yanagisawa, T.; Sakamoto, K.; Yokoyama, S., Adding l-lysine derivatives to the genetic code of mammalian cells with engineered pyrrolysyl-tRNA synthetases. Biochem Biophys Res Commun 2008, 371 (4), 818-22.
26. Hancock, S. M.; Uprety, R.; Deiters, A.; Chin, J. W., Expanding the genetic code of yeast for incorporation of diverse unnatural amino acids via a pyrrolysyl-tRNA synthetase/tRNA pair. J Am Chem Soc 2010, 132 (42), 14819-24.
27. Wang, L.; Schultz, P. G., A general approach for the generation of orthogonal tRNAs. Chem Biol 2001, (9), 883-90.
28. Rogerson, D. T.; Sachdeva, A.; Wang, K.; Haq, T.; Kazlauskaite, A.; Hancock, S. M.; Huguenin-Dezot, N.; Muqit, M. M.; Fry, A. M.; Bayliss, R.; Chin, J. W., Efficient genetic encoding of phosphoserine and its nonhydrolyzable analog. Nat Chem Biol 2015, 11 (7), 496-503.
29. Hughes, R. A.; Ellington, A. D., Rational design of an orthogonal tryptophanyl nonsense suppressor tRNA. Nucleic Acids Res 2010, 38 (19), 6813-30.
30. Ellefson, J. W.; Meyer, A. J.; Hughes, R. A.; Cannon, J. R.; Brodbelt, J. S.; Ellington, A. D., Directed evolution of genetic parts and circuits by compartmentalized partnered replication. Nat Biotechnol 2014, 32 (1), 97-101.
31. Dieci, G.; Fiorino, G.; Castelnuovo, M.; Teichmann, M.; Pagano, A., The expanding RNA polymerase IIItranscriptome. Trends Genet 2007, 23 (12), 614-22.
32. Gonos, E. S.; Goddard, J. P., The role of the 5′-flanking sequence of a human tRNA (Glu) gene in modulation of its transcriptional activity in vitro. Biochem J 1990, 272 (3), 797-803.
33. Schaack, J.; Sharp, S.; Dingermann, T.; Burke, D. J.; Cooley, L.; Soll, D., The extent of a eukaryotic tRNA gene. 5′- and 3′-flanking sequence dependence for transcription and stable complex formation. J Biol Chem 1984,259 (3), 1461-7.
34. Walker, S. C.; Engelke, D. R., Ribonuclease P: the evolution of an ancient RNA enzyme. Crit Rev Biochem Mol Biol 2006, 41 (2), 77-102.
35. Levinger, L.; Vasisht, V.; Greene, V.; Bourne, R.; Birk, A.; Kolla, S., Sequence and structure requirementsfor Drosophila tRNA 5′- and 3′-end processing. J Biol Chem 1995, 270 (32), 18903-9.
36. Piper, P. W.; Straby, K. B., Processing of transcripts of a dimeric tRNA gene in yeast uses the nuclease responsible for maturation of the 3′ termini upon 5 S and 37 S precursor rRNAs. FEBS Lett 1989, 250 (2), 311-6.
37. Copela, L. A.; Fernandez, C. F.; Sherrer, R. L.; Wolin, S. L., Competition between the Rex1 exonuclease and the La protein affects both Trf4p-mediated RNA quality control and pre-tRNA maturation. Rna 2008, 14 (6), 1214-27.
38. Ozanick, S. G.; Wang, X.; Costanzo, M.; Brost, R. L.; Boone, C.; Anderson, J. T., Rexlp deficiency leads to accumulation of precursor initiator tRNAMet and polyadenylation of substrate RNAs in Saccharomyces cerevisiae. Nucleic Acids Res 2009, 37 (1), 298-308.
39. Vogel, A.; Schilling, O.; Spath, B.; Marchfelder, A., The tRNase Z family of proteins: physiological functions, substrate specificity and structural properties. Biol Chem 2005, 386 (12), 1253-64.
40. Nashimoto, M., Distribution of both lengths and 5′ terminal nucleotides of mammalian pre-tRNA 3′trailers reflects properties of 3′ processing endoribonuclease. Nucleic Acids Res 1997, 25 (6), 1148-54.
41. Phizicky, E. M.; Hopper, A. K., tRNA biology charges to the front. Genes Dev 2010, 24 (17), 1832-60.
42. Perona, J. J.; Hadd, A., Structural diversity and protein engineering of the aminoacyl-tRNA synthetases. Biochemistry 2012, 51 (44), 8705-29.
43. Chatterjee, A.; Xiao, H.; Yang, P. Y.; Soundararajan, G.; Schultz, P. G., A tryptophanyl-tRNA synthetase/tRNA pair for unnatural amino acid mutagenesis in E. coli. Angew Chem Int Ed Engl 2013, 52 (19), 5106-9.
44. LaRiviere, F. J.; Wolfson, A. D.; Uhlenbeck, O. C., Uniform binding of aminoacyl-tRNAs to elongation factor Tu by thermodynamic compensation. Science 2001, 294 (5540), 165-8.
45. Saks, M. E.; Sanderson, L. E.; Choi, D. S.; Crosby, C. M.; Uhlenbeck, O. C., Functional consequences of T-stem mutations in E. coli tRNAThrUGU in vitro and in vivo. RNA 2011, 17 (6), 1038-47.
46. Schrader, J. M.; Chapman, S. J.; Uhlenbeck, O. C., Tuning the affinity of aminoacyl-tRNA to elongationfactor Tu for optimal decoding. Proc Natl Acad Sci USA 2011, 108 (13), 5215-20.
47. Albers, J.; Danzer, C.; Rechsteiner, M.; Lehmann, H.; Brandt, L. P.; Hejhal, T.; Catalano, A.; Busenhart,P.; Goncalves, A. F.; Brandt, S.; Bode, P. K.; Bode-Lesniewska, B.; Wild, P. J.; Frew, I. J., A versatile modular vector system for rapid combinatorial mammalian genetics. J Clin Invest 2015, 125 (4), 1603-19.
48. Kurosaki, T.; Maquat, L. E., Nonsense-mediated mRNA decay in humans at a glance. J Cell Sci 2016, 129 (3), 461-7.
49. Asahara, H.; Uhlenbeck, O. C., The tRNA specificity of Thermus thermophilus EF-Tu. Proc Natl Acad Sci USA 2002, 99 (6), 3499-504.
50. Dale, T.; Sanderson, L. E.; Uhlenbeck, O. C., The affinity of elongation factor Tu for an aminoacyl-tRNAis modulated by the esterified amino acid. Biochemistry 2004, 43 (20), 6159-66.
51. Sanderson, L. E.; Uhlenbeck, O. C., The 51-63 base pair of tRNA confers specificity for binding by EF-Tu. Rna 2007, 13 (6), 835-40.
52. Schrader, J. M.; Chapman, S. J.; Uhlenbeck, O. C., Understanding the sequence specificity of tRNA binding to elongation factor Tu using tRNA mutagenesis. J Mol Biol 2009, 386 (5), 1255-64.
53. Zeng, W.; Vu, G. P.; Bai, Y.; Chen, Y. C.; Trang, P.; Lu, S.; Xiao, G.; Liu, F., RNase P-associated external guide sequence effectively reduces the expression of human CC-chemokine receptor 5 and inhibits the infectionof human immunodeficiency virus 1. Biomed Res Int 2013, 2013, 509714.
54. Zhang, Z.; Ye, Y.; Gong, J.; Ruan, H.; Liu, C. J.; Xiang, Y.; Cai, C.; Guo, A. Y.; Ling, J.; Diao, L.; Weinstein, J. N.; Han, L., Global analysis of tRNA and translation factor expression reveals a dynamic landscape of translational regulation in human cancers. Commun Biol 2018, 1, 234.

The foregoing examples and description of the preferred embodiments should be taken as illustrating, rather than as limiting the present disclosure as defined by the claims. As will be readily appreciated, numerous variations and combinations of the features set forth above can be utilized without departing from the present disclosure as set forth in the claims. Such variations are not regarded as a departure from the scope of the disclosure, and all such variations are intended to be included within the scope of the following claims. All references cited herein are incorporated by reference in their entireties.

1. A non-natural nucleic acid comprising:
a 5′ leader segment comprising (i) nucleotides 1 to 55 of a modified sequence selected from SEQ ID NOs: 1-39 and 41-79 as shown in Table 1A or 1B or (ii) one selected from SEQ ID NOs: 81-90 as shown in Table 1C;
a coding segment encoding a transfer RNA (tRNA), and
a 3′ trailer segment.

2. A non-natural nucleic acid comprising:
a 5′ leader segment;
a coding segment encoding a tRNA, and
a 3′ trailer segment comprising nucleotides 129 to 132 of a modified sequence selected from SEQ ID NOs: 92-95 and 97-106 as shown in Table 2A or 2B.

3. A non-natural nucleic acid comprising:
a 5′ leader segment as recited in claim 1;
a coding segment encoding a tRNA, and
a 3′ trailer segment comprising nucleotides 129 to 132 of a modified sequence selected from SEQ ID NOs: 92-95 and 97-106 as shown in Table 2A or 2B.

4. The nucleic acid of claim 1, comprising a modified sequence selected from SEQ ID NOs: 1-39, 41-79, 81-90, 92-95, 97-106, 152, 153-162, 164-177, 179-194, 196-210, and 228-251 shown in Table 1A, 1B, 1C, 2A, 2B, 9, or 10.

5. The nucleic acid of claim 1, further comprising a tabulator sequence.

6. The nucleic acid of claim 5, wherein the tabulator sequence encodes a ribozyme.

7. A non-natural nucleic acid comprising nucleotides 56-128 of a modified sequence selected from SEQ ID NOs: 108-111, 113-116, 118-131, and 133-146 as shown in Tables 3 to 7.

8. The nucleic acid of claim 1, further comprising an upstream control element (UCE) operably linked to the 5′ end of the 5′ leader segment.

9. The nucleic acid of claim 8, wherein the upstream control element comprises a U6 promoter or an H1 promoter or a sequence selected from SEQ ID NOs: 148-151 shown in Table 8.

10. The nucleic acid of claim 1, wherein the tRNA is an anti-codon edited-tRNA (ACE-tRNA).

11. An expression cassette or vector comprising the nucleic acid of claim 1.

12. A host cell comprising the nucleic acid of claim 1 or an expression cassette or vector comprising said nucleic acid, or a progeny of the host cell.

13. A tRNA encoded by the nucleic acid of claim 1.

14. A pharmaceutical formulation comprising (i) the nucleic acid of claim 1, or an expression cassette or vector comprising said nucleic acid, or a tRNA encoded by said nucleic acid, and (ii) a pharmaceutically acceptable carrier.

15. A method for expressing or introducing a tRNA in a cell, comprising
(i) contacting the cell with the nucleic acid of claim 1, or with an expression cassette or vector comprising said nucleic acid, or with a tRNA encoded by said nucleic acid, or with a pharmaceutical formulation comprising said nucleic acid, or said expression cassette or vector, or said tRNA, and
(ii) maintaining the cell under conditions permitting expression of the tRNA.

16. A method of treating a disease associated with a PTC in a subject in need thereof, the method comprising administering to the subject
(i) the nucleic acid of claim 10, or
(ii) an expression cassette or vector comprising said nucleic acid, or
(iii) a pharmaceutical composition comprising said nucleic acid or said expression cassette or vector.

17. The method of claim 16, wherein the disease is selected from the group consisting of cystic fibrosis, Duchenne and Becker muscular dystrophies, retinoblastoma, neurofibromatosis, ataxia-telangiectasia, Tay-Sachs disease, Wilm's tumor, hemophilia A, hemophilia B, Menkes disease, Ullrich's disease, β-Thalassemia, type 2A and type 3 von Willebrand disease, Robinow syndrome, brachydactyly type B (shortening of digits and metacarpals), inherited susceptibility to mycobacterial infection, inherited retinal disease, inherited bleeding tendency, inherited blindness, congenital neurosensory deafness and colonic agangliosis and inherited neural develop-mental defect including neurosensory deafness, colonic agangliosis, peripheral neuropathy and central dysmyelinating leukodystrophy, Liddle's syndrome, xeroderma pigmentosum, Fanconi's anemia, anemia, hypothyroidism, p53-associated cancers, esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes, rickets, Hurler Syndrome, Dravet Syndrome, Spinal Muscular Dystrophy, Usher Syndrome, Aniridia, Choroideremia, Ocular Coloboma, Retinitis pigmentosa, dystrophic epidermolysis bullosa, Pseudoxanthoma elasticum, Alagille Snydrome, Waardenburg-Shah, infantile neuronal ceroid lipofuscinosis, Cystinosis, X-linked nephrogenic diabetes insipidus, McArdle's disease and Polycystic kidney disease.

18. The method of claim 16, wherein the disease is an ocular genetic disease selected from the group consisting of cone dystrophies, Stargardt's disease (STGD1), cone-rod dystrophy, retinitis pigmentosa (RP), increased susceptibility to age-related macular degeneration, Congenital stationary night blindness 2 (CSNB2), Congenital stationary night blindness 1 (CSNB1), Best Disease, VMD, and Leber congenital amaurosis (LCA16).

19. The method of claim 16, wherein the administering is carried out using viral delivery, nanoparticles, electroporation, polyethylenimine (PEI), receptor-targeted polyplexes, liposomes, or hydrodynamic injection.


SEQUENCE LISTING (265 sequences)

SEQ ID NO: 1 - DNA, 139 bp, synthetic construct
  source 1..139: mol_type = other DNA
gatgaggttgcggtaagcattagagggctatcagcagcatcttatcgcagcggagggctctgtggcgcaatggatagcgcattggacttcaaattcaaaggttgtgggttcgagtcccaccagagtcggtccttttttt

SEQ ID NO: 2 - DNA, 139 bp, synthetic construct
  source 1..139: mol_type = other DNA
atagcgtttagacaagagacagaagtgagtcctgagcacaatgtagcataagcgcggctctgtggcgcaatggatagcgcattggacttcaaattcaaaggttgtgggttcgagtcccaccagagtcggtccttttttt

SEQ ID NO: 3 - DNA, 139 bp, synthetic construct
  source 1..139: mol_type = other DNA
ggtgagaggcggaacaaggctgaagggaagctctgggagtgcccttgccgagagcggctctgtggcgcaatggatagcgcattggacttcaaattcaaaggttgtgggttcgagtcccaccagagtcggtccttttttt

SEQ ID NO: 4 - DNA, 139 bp, synthetic construct
  source 1..139: mol_type = other DNA
gcgtatgtgttgagttctcgcttcataaatatgttttaataaacctacttcagctggctctgtggcgcaatggatagcgcattggacttcaaattcaaaggttgtgggttcgagtcccaccagagtcggtccttttttt

SEQ ID NO: 5 - DNA, 139 bp, synthetic construct
  source 1..139: mol_type = other DNA
acgcttgccgctgtcatcgcctctagtaaattcgaggagaccttgctcagcagatggctctgtggcgcaatggatagcgcattggacttcaaattcaaaggttgtgggttcgagtcccaccagagtcggtccttttttt

SEQ ID NO: 6 - DNA, 139 bp, synthetic construct
  source 1..139: mol_type = other DNA
agtttcgtgcgtgcgcccttcctatagcgcccagtagaactgactgacagtacctggctctgtggcgcaatggatagcgcattggacttcaaattcaaaggttgtgggttcgagtcccaccagagtcggtccttttttt

SEQ ID NO: 7 - DNA, 139 bp, synthetic construct
  source 1..139: mol_type = other DNA
gcgggcgcgggcttggagccctcttaggcgctcccgtgggggcccaacgtcaggcggctctgtggcgcaatggatagcgcattggacttcaaattcaaaggttgtgggttcgagtcccaccagagtcggtccttttttt

SEQ ID NO: 8 - DNA, 139 bp, synthetic construct
  source 1..139: mol_type = other DNA
aaccatgtagttaaacagtcctcaagaagtaaaaaagcaggctaacgtaacaggcggctctgtggcgcaatggatagcgcattggacttcaaattcaaaggttgtgggttcgagtcccaccagagtcggtccttttttt

SEQ ID NO: 9 - DNA, 139 bp, synthetic construct
  source 1..139: mol_type = other DNA
atcattgcttgttgatctctattttagtgcaaaaatcagggctgttatgcagtgtggctctgtggcgcaatggatagcgcattggacttcaaattcaaaggttgtgggttcgagtcccaccagagtcggtccttttttt

SEQ ID NO: 10 - DNA, 139 bp, synthetic construct
  source 1..139: mol_type = other DNA
cggggcggagcaaaaacgtagaaaaggcctatcagaaggctttctttcgtgcggtggctctgtggcgcaatggatagcgcattggacttcaaattcaaaggttgtgggttcgagtcccaccagagtcggtccttttttt

SEQ ID NO: 11 - DNA, 139 bp, synthetic construct
  source 1..139: mol_type = other DNA
tgtcctgagttaagcgaccaatagggaccatctgggagagctgctgagagtaggtggctctgtggcgcaatggatagcgcattggacttcaaattcaaaggttgtgggttcgagtcccaccagagtcggtccttttttt

SEQ ID NO: 12 - DNA, 139 bp, synthetic construct
  source 1..139: mol_type = other DNA
gccaggggtgtggccatacaggtttatagtggttagtagagacaagtaagtgggtggctctgtggcgcaatggatagcgcattggacttcaaattcaaaggttgtgggttcgagtcccaccagagtcggtccttttttt

SEQ ID NO: 13 - DNA, 139 bp, synthetic construct
  source 1..139: mol_type = other DNA
tgggggcggggttctctgaggattagacagccgaaggcagctacacatgcgtggcggctctgtggcgcaatggatagcgcattggacttcaaattcaaaggttgtgggttcgagtcccaccagagtcggtccttttttt

SEQ ID NO: 14 - DNA, 139 bp, synthetic construct
  source 1..139: mol_type = other DNA
aaaccctgcagtataaccttgaagtaccatcacggagagaaacagtgccatctcaggctctgtggcgcaatggatagcgcattggacttcaaattcaaaggttgtgggttcgagtcccaccagagtcggtccttttttt

SEQ ID NO: 15 - DNA, 139 bp, synthetic construct
  source 1..139: mol_type = other DNA
ggggcgcctccgtcatcacactaatatgccggcataggaagccctacgatcgggaggctctgtggcgcaatggatagcgcattggacttcaaattcaaaggttgtgggttcgagtcccaccagagtcggtccttttttt

SEQ ID NO: 16 - DNA, 139 bp, synthetic construct
  source 1..139: mol_type = other DNA
acgcattcattacaatcgaagattatattaatttgcgatggtaagtgtcccaggtggctctgtggcgcaatggatagcgcattggacttcaaattcaaaggttgtgggttcgagtcccaccagagtcggtccttttttt

SEQ ID NO: 17 - DNA, 139 bp, synthetic construct
  source 1..139: mol_type = other DNA
gaggacttcccgcgtcacagtctcttgtctccgcgggcagcaactgtcttagacaggctctgtggcgcaatggatagcgcattggacttcaaattcaaaggttgtgggttcgagtcccaccagagtcggtccttttttt

SEQ ID NO: 18 - DNA, 139 bp, synthetic construct
  source 1..139: mol_type = other DNA
tcaatctgtaagagcatcttctcttatacctttccaaatccttattatttctgtgggctctgtggcgcaatggatagcgcattggacttcaaattcaaaggttgtgggttcgagtcccaccagagtcggtccttttttt

SEQ ID NO: 19 - DNA, 139 bp, synthetic construct
  source 1..139: mol_type = other DNA
gcgcctgttttgaggattctgagtattagggtgggaagcaagtattattacatcaggctctgtggcgcaatggatagcgcattggacttcaaattcaaaggttgtgggttcgagtcccaccagagtcggtccttttttt

SEQ ID NO: 20 - DNA, 139 bp, synthetic construct
  source 1..139: mol_type = other DNA
ccaaagaaattatgtattctcttagtgtattgggggaagagctacgttcgggtgaggctctgtggcgcaatggatagcgcattggacttcaaattcaaaggttgtgggttcgagtcccaccagagtcggtccttttttt

SEQ ID NO: 21 - DNA, 139 bp, synthetic construct
  source 1..139: mol_type = other DNA
atcaaaacaatcttattcctttgaaaagatctaggaacaacacttttctctaactggctctgtggcgcaatggatagcgcattggacttcaaattcaaaggttgtgggttcgagtcccaccagagtcggtccttttttt

SEQ ID NO: 22 - DNA, 139 bp, synthetic construct
  source 1..139: mol_type = other DNA
attaccagggtgtaagtgtcttctagtaagcaatagaaaagcacatttgctataaggctctgtggcgcaatggatagcgcattggacttcaaattcaaaggttgtgggttcgagtcccaccagagtcggtccttttttt

SEQ ID NO: 23 - DNA, 139 bp, synthetic construct
  source 1..139: mol_type = other DNA
tccgaaacactgatgaaattcttgatgcagtttttaaaacttcttggagacaggtggctctgtggcgcaatggatagcgcattggacttcaaattcaaaggttgtgggttcgagtcccaccagagtcggtccttttttt

SEQ ID NO: 24 - DNA, 139 bp, synthetic construct
  source 1..139: mol_type = other DNA
atgtggctccctctgtttgcagaaagtccaatgaaccagctttgatagcatgcatggctctgtggcgcaatggatagcgcattggacttcaaattcaaaggttgtgggttcgagtcccaccagagtcggtccttttttt

SEQ ID NO: 25 - DNA, 139 bp, synthetic construct
  source 1..139: mol_type = other DNA
gtcagcgtctgctgactacgagttacataatgaaacttaaggagtaacagtgcgtggctctgtggcgcaatggatagcgcattggacttcaaattcaaaggttgtgggttcgagtcccaccagagtcggtccttttttt

SEQ ID NO: 26 - DNA, 139 bp, synthetic construct
  source 1..139: mol_type = other DNA
atacttgacaagagtaatgttgaaacagcagagtggcggagcggactgtcgagacggctctgtggcgcaatggatagcgcattggacttcaaattcaaaggttgtgggttcgagtcccaccagagtcggtccttttttt

SEQ ID NO: 27 - DNA, 139 bp, synthetic construct
  source 1..139: mol_type = other DNA
cactgtgcccagcctcatggcctcatagtctagctttgaaactcagctagcctgtggctctgtggcgcaatggatagcgcattggacttcaaattcaaaggttgtgggttcgagtcccaccagagtcggtccttttttt

SEQ ID NO: 28 - DNA, 139 bp, synthetic construct
  source 1..139: mol_type = other DNA
taaatgcaaaagcaaaagcattaagtagctttctggcataaacatattgcagctgggctctgtggcgcaatggatagcgcattggacttcaaattcaaaggttgtgggttcgagtcccaccagagtcggtccttttttt

SEQ ID NO: 29 - DNA, 139 bp, synthetic construct; identical to SEQ ID NO: 28
  source 1..139: mol_type = other DNA

SEQ ID NO: 30 - DNA, 139 bp, synthetic construct
  source 1..139: mol_type = other DNA
cggggagggtaaaagaggaaagggtagagaataggttcccaactgttagccagtaggctctgtggcgcaatggatagcgcattggacttcaaattcaaaggttgtgggttcgagtcccaccagagtcggtccttttttt

SEQ ID NO: 31 - DNA, 139 bp, synthetic construct
  source 1..139: mol_type = other DNA
caaacccatgatcccccactattaaggatatccggagaggatgctacctatcaggggctctgtggcgcaatggatagcgcattggacttcaaattcaaaggttgtgggttcgagtcccaccagagtcggtccttttttt

SEQ ID NO: 32 - DNA, 139 bp, synthetic construct
  source 1..139: mol_type = other DNA
accttccagtcgcgttccaatcatgttactgcttttccccaactcttctatcggtggctctgtggcgcaatggatagcgcattggacttcaaattcaaaggttgtgggttcgagtcccaccagagtcggtccttttttt

SEQ ID NO: 33 - DNA, 139 bp, synthetic construct
  source 1..139: mol_type = other DNA
acggaagtgtgaaatagttttcaagtagtatacgatcgagggctttctcggctgtggctctgtggcgcaatggatagcgcattggacttcaaattcaaaggttgtgggttcgagtcccaccagagtcggtccttttttt

SEQ ID NO: 34 - DNA, 139 bp, synthetic construct
  source 1..139: mol_type = other DNA
caagggcaggcggaaatgattcaattggtttagaattcaacggttacttgtcatcggctctgtggcgcaatggatagcgcattggacttcaaattcaaaggttgtgggttcgagtcccaccagagtcggtccttttttt

SEQ ID NO: 35 - DNA, 139 bp, synthetic construct
  source 1..139: mol_type = other DNA
cggcttgcgtgcggctgactacgaagacgtgagtgacgctttcctcccgtgtgacggctctgtggcgcaatggatagcgcattggacttcaaattcaaaggttgtgggttcgagtcccaccagagtcggtccttttttt

SEQ ID NO: 36 - DNA, 139 bp, synthetic construct
  source 1..139: mol_type = other DNA
aacgcatctcctgtaagtagttaagagtactgtgagaccgtgtgcctggcagaacggctctgtggcgcaatggatagcgcattggacttcaaattcaaaggttgtgggttcgagtcccaccagagtcggtccttttttt

SEQ ID NO: 37 - DNA, 139 bp, synthetic construct
  source 1..139: mol_type = other DNA
agaactgaacagtattgtaactaaatagatttatttcaagcttttcgtagcaactggctctgtggcgcaatggatagcgcattggacttcaaattcaaaggttgtgggttcgagtcccaccagagtcggtccttttttt

SEQ ID NO: 38 - DNA, 139 bp, synthetic construct
  source 1..139: mol_type = other DNA
aatggcacactgctcacatcttcaaaagcttaaaccaagagcactacacaggtgcggctctgtggcgcaatggatagcgcattggacttcaaattcaaaggttgtgggttcgagtcccaccagagtcggtccttttttt

SEQ ID NO: 39 - DNA, 139 bp, synthetic construct
  source 1..139: mol_type = other DNA
tacaatatgtgatgtatggaaacatgtaagacatttaataaggtttttggtatctggctctgtggcgcaatggatagcgcattggacttcaaattcaaaggttgtgggttcgagtcccaccagagtcggtccttttttt

SEQ ID NO: 40 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttgtgggttcgagtcccaccagagtcggtccttttttt

SEQ ID NO: 41 - DNA, 139 bp, synthetic construct; identical to SEQ ID NO: 12
  source 1..139: mol_type = other DNA

SEQ ID NO: 42 - DNA, 139 bp, synthetic construct; identical to SEQ ID NO: 18
  source 1..139: mol_type = other DNA

SEQ ID NO: 43 - DNA, 139 bp, synthetic construct; identical to SEQ ID NO: 1
  source 1..139: mol_type = other DNA

SEQ ID NO: 44 - DNA, 139 bp, synthetic construct; identical to SEQ ID NO: 11
  source 1..139: mol_type = other DNA

SEQ ID NO: 45 - DNA, 139 bp, synthetic construct; identical to SEQ ID NO: 2
  source 1..139: mol_type = other DNA

SEQ ID NO: 46 - DNA, 139 bp, synthetic construct; identical to SEQ ID NO: 28
  source 1..139: mol_type = other DNA

SEQ ID NO: 47 - DNA, 139 bp, synthetic construct; identical to SEQ ID NO: 5
  source 1..139: mol_type = other DNA

SEQ ID NO: 48 - DNA, 139 bp, synthetic construct
  source 1..139: mol_type = other DNA
aaagaataggaattgccaaatgaaactgttgagaagagaaacattacacatatcaggctctgtggcgcaatggatagcgcattggacttcaaattcaaaggttgtgggttcgagtcccaccagagtcggtccttttttt

SEQ ID NO: 49 - DNA, 139 bp, synthetic construct; identical to SEQ ID NO: 38
  source 1..139: mol_type = other DNA

SEQ ID NO: 50 - DNA, 139 bp, synthetic construct
  source 1..139: mol_type = other DNA
ctggcaccagcggacacaagtatgaggctaagtgagaagcgctctgcttccgagtggctctgtggcgcaatggatagcgcattggacttcaaattcaaaggttgtgggttcgagtcccaccagagtcggtccttttttt

SEQ ID NO: 51 - DNA, 139 bp, synthetic construct
  source 1..139: mol_type = other DNA
aggaggaggtagaggatgccttgtaagcggagcaaaaacaaggttcaacgtctgcggctctgtggcgcaatggatagcgcattggacttcaaattcaaaggttgtgggttcgagtcccaccagagtcggtccttttttt

SEQ ID NO: 52 - DNA, 139 bp, synthetic construct; identical to SEQ ID NO: 16
  source 1..139: mol_type = other DNA

SEQ ID NO: 53 - DNA, 139 bp, synthetic construct; identical to SEQ ID NO: 3
  source 1..139: mol_type = other DNA

SEQ ID NO: 54 - DNA, 139 bp, synthetic construct; identical to SEQ ID NO: 15
  source 1..139: mol_type = other DNA

SEQ ID NO: 55 - DNA, 139 bp, synthetic construct; identical to SEQ ID NO: 8
  source 1..139: mol_type = other DNA

SEQ ID NO: 56 - DNA, 139 bp, synthetic construct
  source 1..139: mol_type = other DNA
agaaccatactgcagaaacgaggtctttagcgatgcggagtcaccactttggtgtggctctgtggcgcaatggatagcgcattggacttcaaattcaaaggttgtgggttcgagtcccaccagagtcggtccttttttt

SEQ ID NO: 57 - DNA, 139 bp, synthetic construct; identical to SEQ ID NO: 33
  source 1..139: mol_type = other DNA

SEQ ID NO: 58 - DNA, 139 bp, synthetic construct; identical to SEQ ID NO: 7
  source 1..139: mol_type = other DNA

SEQ ID NO: 59 - DNA, 139 bp, synthetic construct; identical to SEQ ID NO: 17
  source 1..139: mol_type = other DNA

SEQ ID NO: 60 - DNA, 139 bp, synthetic construct; identical to SEQ ID NO: 25
  source 1..139: mol_type = other DNA

SEQ ID NO: 61 - DNA, 139 bp, synthetic construct
  source 1..139: mol_type = other DNA
cttgattgtattgagtcaccataagtggacaaggaaggaacctctaacagttgaaggctctgtggcgcaatggatagcgcattggacttcaaattcaaaggttgtgggttcgagtcccaccagagtcggtccttttttt

SEQ ID NO: 62 - DNA, 139 bp, synthetic construct; identical to SEQ ID NO: 10
  source 1..139: mol_type = other DNA

SEQ ID NO: 63 - DNA, 139 bp, synthetic construct; identical to SEQ ID NO: 9
  source 1..139: mol_type = other DNA

SEQ ID NO: 64 - DNA, 139 bp, synthetic construct; identical to SEQ ID NO: 13
  source 1..139: mol_type = other DNA

SEQ ID NO: 65 - DNA, 139 bp, synthetic construct
  source 1..139: mol_type = other DNA
tgccccattcccactctagtacccgtaagctacaagacgccgccgttcgtcgggtggctctgtggcgcaatggatagcgcattggacttcaaattcaaaggttgtgggttcgagtcccaccagagtcggtccttttttt

SEQ ID NO: 66 - DNA, 139 bp, synthetic construct
  source 1..139: mol_type = other DNA
ccttgggtgtggcaacacaggtgaaatatgctaaggaggaatgtccaccaggcaaggctctgtggcgcaatggatagcgcattggacttcaaattcaaaggttgtgggttcgagtcccaccagagtcggtccttttttt

SEQ ID NO: 67 - DNA, 139 bp, synthetic construct; identical to SEQ ID NO: 30
  source 1..139: mol_type = other DNA

SEQ ID NO: 68 - DNA, 139 bp, synthetic construct
  source 1..139: mol_type = other DNA
tcacggcataatggtaacagagtcttcattaatggaagcaacgttactaccgtgaggctctgtggcgcaatggatagcgcattggacttcaaattcaaaggttgtgggttcgagtcccaccagagtcggtccttttttt

SEQ ID NO: 69 - DNA, 139 bp, synthetic construct; identical to SEQ ID NO: 6
  source 1..139: mol_type = other DNA

SEQ ID NO: 70 - DNA, 139 bp, synthetic construct; identical to SEQ ID NO: 39
  source 1..139: mol_type = other DNA

SEQ ID NO: 71 - DNA, 139 bp, synthetic construct; identical to SEQ ID NO: 23
  source 1..139: mol_type = other DNA

SEQ ID NO: 72 - DNA, 139 bp, synthetic construct; identical to SEQ ID NO: 21
  source 1..139: mol_type = other DNA

SEQ ID NO: 73 - DNA, 139 bp, synthetic construct
  source 1..139: mol_type = other DNA
acagcaggcggaaaagagaaattcagagtgataggaagcaccactcagctacagtggctctgtggcgcaatggatagcgcattggacttcaaattcaaaggttgtgggttcgagtcccaccagagtcggtccttttttt

SEQ ID NO: 74 - DNA, 139 bp, synthetic construct
  source 1..139: mol_type = other DNA
gagccgtaagccgtgattactcatcatgtatagtttcaaggatttttgtcacagtggctctgtggcgcaatggatagcgcattggacttcaaattcaaaggttgtgggttcgagtcccaccagagtcggtccttttttt

SEQ ID NO: 75 - DNA, 139 bp, synthetic construct
  source 1..139: mol_type = other DNA
gtaaatttctatattctttcataaaagcagtttaaagacgaacgtttttcgaggtggctctgtggcgcaatggatagcgcattggacttcaaattcaaaggttgtgggttcgagtcccaccagagtcggtccttttttt

SEQ ID NO: 76 - DNA, 139 bp, synthetic construct
  source 1..139: mol_type = other DNA
tggcttttcctatagagaaccctcttataagcaaaaagtaaagctctcgtgaagaggctctgtggcgcaatggatagcgcattggacttcaaattcaaaggttgtgggttcgagtcccaccagagtcggtccttttttt

SEQ ID NO: 77 - DNA, 139 bp, synthetic construct
  source 1..139: mol_type = other DNA
caaaatgtctttttgaacaattatcaaatcctggaggaacgttttagtcgtgtggggctctgtggcgcaatggatagcgcattggacttcaaattcaaaggttgtgggttcgagtcccaccagagtcggtccttttttt

SEQ ID NO: 78 - DNA, 139 bp, synthetic construct; identical to SEQ ID NO: 35
  source 1..139: mol_type = other DNA

SEQ ID NO: 79 - DNA, 139 bp, synthetic construct
  source 1..139: mol_type = other DNA
cagaggcagggagattaggggaaagggagaatggggagaaactgagtaatgggtaggctctgtggcgcaatggatagcgcattggacttcaaattcaaaggttgtgggttcgagtcccaccagagtcggtccttttttt

SEQ ID NO: 80 - DNA, 139 bp, synthetic construct; identical to SEQ ID NO: 40
  source 1..139: mol_type = other DNA

SEQ ID NO: 81 - DNA, 853 bp, synthetic construct
  source 1..853: mol_type = other DNA
tttccagccccctgagatcctagaaatccttctggagaagggcaccctctcggtaccgaggtcttctggtccctggaaacagcctgtgcgcggcgtgcgtaagcctagagggggtggaagggaagagaggaagatgccgcgggggccaggaaaagagagatgagtcacgaaatgaggggatgcaggaaggaggaagcgagggaagaatgaatccatgggatcttccctgcgtacctactgtgtgccgggtacggtttgggcactggggacataggaatagggcgggtctggctgtgcagctgcaggcctcgttctatcccggagcctgagtaaatgaattataacacatatacaaatgtgaagctgcagctggggcgagtattcggaaggagagcgcggtaagagtgccaggcggccttgcaatacctgggctttacctagattgggcgccagcccacggaaatgtctgcgaatcagtgcaggtatacggacttgattgcattgtgcacaagggaaatgcaaggttgcttgcagatttcaggaatcaaagctgacatgatagtacatgtatgaatgcagaatgaatacaggctggaggcacgaatgcatgtgtattgaagtaacagtcttcggtctcggccttctctagtaggccttgcgcaccctctggcgtcgcctaggtgcatcgcgctgaccttccttgtcccatctctccacgcccattggagcaagtgagtgtatctcgctccacccaacgccgggcggacaactaggagtgggtcttcgctgcccactacgcatgcgcatattgcagaacttgtgaagaggctttatgcgccacacactgcaagca

SEQ ID NO: 82 - DNA, 851 bp, synthetic construct
  source 1..851: mol_type = other DNA
tttgaaagggccaacttagctcctgcccccgcagagcttccagtctgagtcgctggaactgggaaagaacagaggactccaggaggttggggttgaccctgagcggagaggaaggacgaaaggtaatcggtaggtgagtgtttgggggtaatgttcgttaatgcaaaaaataagaaaaacccgcacagccaggcacaagtgcttgccctccgcgccagaccctcgagcgcactcgcgctccacttttgcatccactgctccaggcccggtgctccctccgccgcgcggccaagaggtctccattttggggtagctgcggaaggctcgcttcttagtcgctgccccgcccctgtagcgttgaggcgtccctcgcctcgctcccactaatccttctgtgtagccctcgtccgcggcctgcgcaccctcccctccggccctggactgacacatcgcttttggggtcgatgcactgtgtccaacattcggcctgaattgattaagcacttgctgtatgccgagcctgaggcagccactgtccgggcgctggaggggggaagcatggatcagggattccaaggcgggggcgggtcccgccggtggttgagggttcgcggggacaggagttgaggggcagggaggaaagggtttctgggtagcatgggggcgccgtacgttcttaggagcaaagtcgggagacattagcaggcctgaatccctgcgtgtccagggagaacgcgtggatttctatgcacagaagccacgcccccacccaactctttcccgcaagcgctggcccacacgcttgccgctgtcatcgcctctagtaaattcgaggagaccttgctcagcag

SEQ ID NO: 83 - DNA, 851 bp, synthetic construct
  source 1..851: mol_type = other DNA
tttaaagacccacaagggaggccagtggcctgtgctgggcactgtacagccgccccgaccccgacctcacgtgggcctcggaggctgcagggacctacccggccgcgcgccgcccaccatcccaggtctaccgcaggggaatcccccaggcttgcgagcgcttccgagggcaggaaagcctgtactgcagccccgccgccctctcctggcgcgtagccctctccccgagccctcgggccacgccgccaggcccgcgatcccagggcggcccggggggttcggcacagcccgataatgcgccgcgccgaagcctgcagcaccccgcggtcgccgtctctcttgcactggtcaggacgaatagagcctgacaaaacctgagttaattggccgagcgaatgtcgttcgcgtcaggagagcacgtccggagaagccaagggaaatgtggagcaggagcagccaaagtggcccatcggctctcgagcagggtttgcgcgtcgcggtcgcaaagcccctgtgagtgcaggtaccgcggcacaagcgctgccccggagctccaggaggcggcggcgggctcggcgttcgcggtccaggagaaaccaccgcggggtgttgggaacattttgttaaaaagccgggaagctcagggccggtgtggcctccgttctggcctgagaacccggactttgggcagcttcttttccttgcctttgggtctccggctttctcagctctgtgaggtcccatgggtctcctgccctggcaggttccttcagctggagaaagcgggggtcatggctggatgaggttgcggtaagcattagagggctatcagcagcatcttatcgcagcgg

SEQ ID NO: 84 - DNA, 851 bp, synthetic construct
  source 1..851: mol_type = other DNA
tttggttctgtggtagatatgaggtcctgggtagtggggaatggagtggaagtcctgggaaaagggataacaggacaaactgaatctcatgtgcacagaaaggaaggacagcattgcaaaacaaggaaggggaacagaaatatctcattgcatcgtagggtagcgggggcaaagaggaggccactgcgctgcatccaagactgtgcagtggcgatggtgccttcagccaccaggaagggcgactttgccacgctcccctgcatgtggcgtggcacagccgacacgcgcatttctacaaagcctccctcccgtgtgacttgtggcacgtgcgaagaagaaaagcggcccctaggggcaggggtccaggaactctgctgctgcccgtgcacctcattccttgaaacctactctgcttagaacaacacaaactaaacgccgccctgtgggccgcgtccctgcaccaccaaccaggcgcctccagaacgcgctcccaggaacccagaagtttgctggtgtgtgtttgcggcgcccgcgcacctcgaaactgaaagcgcaactggagggcctggagtctcgccccagccaaaggcccgcgagcgccgctgccgcggcctccaccccaaccgagagcgcgccgctccgctgcagttgtcttcactgccgcagcgggccgggggcaacagtgagccgcggcggcgagaaaccgaggtaccggcgtggcggccgccccagcccgggctgagagagccgagacagtggcggcgaaaactctctgcgttctggagggagggtgcgggcaggaggaggtagaggatgccttgtaagcggagcaaaaacaaggttcaacgtct

SEQ ID NO: 85 - DNA, 851 bp, synthetic construct
  source 1..851: mol_type = other DNA
ttttgccctggtcgttggccaggatctccacgcggccctgctgaaacacgcccacgcacgagtaggtggtgcccaggtcgatgcccaccgcgagctcccgtggggcctgcatggctgaagcttcttgtcggatgctggaggccacggaggctgctgccgctccgccgtcgaggggcgctcagcagtgaggctctccctgcggtttctctgcagccagcccggctcggatctgctcagctccgcttccaccggctttttatccttttgcgcctcacccgacccccgccgccgcgaactttcgcgaacctttccgcaccttcccgcaccttcccgcccagttgagagcctctcgcccgcccgggctgactcagccgggtggcgcggcgcggcgctgaccccgggagaaggttcgggcagatcggggcatgagccgcgaggctgggcggctacgccaggccagcccgcctctgactcaggctgctgaaagaaatgcggggaaggttctagctcctccgggctggctctgaggacgcgcggttgtggcagaggctgcaaaagcagcaactttagaggcaaggtctccccggggctgtgggaggtcggatccctgaggcacacaggggcctgcgggctgccttggagggccgctgagagcccagcggctcaggaaggccgaagatatgttaccatgcgaatacagatatataagatatatacacgtatatcttaaaacagttttatcttgaacaaaatattttccaaaccgcgatttaagacggtgcctggttccttagtgaatggccagagatagtgagctttaagagacaatactgcaggtctttagctgacag

SEQ ID NO: 86 - DNA, 851 bp, synthetic construct
  source 1..851: mol_type = other DNA
tttgcacagcgtgcatcaatccagaaataggctgagataggagcttagtttcctattgcacaccatgctgggtgaacctttctgtgcatgctgcagggatgctgggtggatgggttggcagtgttcggcttcgtagttgcccacaggccctctggaaagtgcggagaccagtggctggtgtagttgcgtgaggacacccagaaggggtcagtgagaaactagggaggggagggccttgcctgctctggcagcagaaacccctccatgtaaggtgttggtgctccacgccagcagcctgggaaccctgaagtccacgatccctcctgagccttccggacccctcgactttcccccagctcaggatccgcggaagtagcgactcaactgaagttatagtcccgggagctggcagggccccgggtcttctcaggctgaggtcgcggggcagaatggtccgaagctcaccggggtcacagtcggaccgaggcgggtgtgcgcctcgaccggacgccaggggagccagcagcccggccgcgctggcggggccgggggttctccggagtgggtaggggctcgaccgcactgccctgagtcccaagccctgcaccagcgttccccaaaggcggagcgtcctggctccacgttggaagagacattaccgtgaccaggaagtctggccttcccaggctccgcccgctccgtgcagcccaagcttccggtctcagtctcagctgcctgtgtgaatcggaagctacactacgcgtgcgccacaacaggccgaatgtgtgcgcatgcgcgcggcccatatggtaaatagtaatgaagaatagtttaggaaagtacctgtacgg

SEQ ID NO: 87 - DNA, 851 bp, synthetic construct
  source 1..851: mol_type = other DNA
tttcaaagggcatgtttgaatctataaatatgagcgtgcacattaaactgtcaggtgccttggggctgacttagccgacaagtcagcaggtctgggcgggagagaagttgcatttctcacaaaatcccaggtgctgctgctggtcttgggaccagggacacatccaggctaaacaggtaaactgggggaagaagaaactttcggctttgtttgtttgtttttatttttttcttttctttgagacaaggtctccctctgtcgctcaggctggtgtgcagcggcgcgatctcggctcactgcagccccgatctcctgggctcaagcgatccttcagcccgggccaaagcgcggagcttccagaagcttttattttctaaatctcacgcctccattcagcaaccctggctgggcgtgggctccctgcaaggcggcggtggacggcccgcagtcgggccgataaggaaacctgctaccattccttggcttcctgggtgcctcactctagagggggaaactgagactccgagtcgagccgtcccgaagcgctacatcgctggcggggctgggattcgaacccgacttctgagatacaggccggtgccccgcccgcgggacctgcgggaccctcgggacaccgctccagccgcgccccgactccagaggttctccagcccactgcagacgacagaatcgcccgcgggtccccgagcccccgagcccgccggggcgcgcccgttgaagccccgccccgccggccgcagcagggagggggacggtgcgagctcgtgctcgctgggcgggcgcacgggagcggcgccaggatagaggttcgcgagaccgccatcgcaacg

SEQ ID NO: 88 - DNA, 851 bp, synthetic construct
  source 1..851: mol_type = other DNA
tttccatttctttgggagatcctcaaataccagtgtttgtcgttgttttaatttttattttttccttgtaaattgacttggatatctcatctgtctttctttatctctggaaaactttatcccagtttcccttgacatactccttcaacctcctgggagaggtagggggaggggagatgagcctgtttgactagttctgggaatgtggtgagggaagagatctggggatttcggctcagtgctgacacttccttcatgctactctggtttagcacccctgcacttcaccctctttcaactcctgaccaagtcaactcctgttactcacatcgacagtcatttttcatgttcaagacccctgaaccgctctcatcccccactctccgctaactctcctgcttcgcttcaggtctacattgcctcgctcatttcttccctgctgagctctttgattagtttgctaagcagcatctcgggaacagcccccgtgctatgtgttctttcttgtcatggatatgtgttttttcttgtcatgctatgtgttctttcttgttcaaggcagagtcgcggctgggttgttaggatgctcgcgcccctgtacccggggcgtgcagggaagtgtaagaagggggcgaagggcgagaaacgctgaattctagcctgaattggtaggagagcctcgcaagctgagtcacggttcctgattccatttaaatagaacgttttgttcacttgtaggaatttccttttccatttctttcattaaatcgcgaacgtagtaatgacttaacgctggttacgcattcattacaatcgaagattatattaatttgcgatggtaagtgtcccag

SEQ ID NO: 89 - DNA, 853 bp, synthetic construct
  source 1..853: mol_type = other DNA
ttttgcggagggagggatcagatgctgagatggagaaaaggaggtaacgcctgtataattgggacactgccctccgcctggtcagcggcccggactgatgccctggagtgcttgtccgcccactccccaatgtcccccaagatctgttcctccccattcccaggcagagatgagcacagggcacctgcattcctacgtgtgaatcatccgtgtgaatcactaaacagtggagagttgcacttggtcataaatcacacgtgactgcagccacacgcaggtgagtcgcacacgcattccagcacatgtcaaatggtcaggaacagctccctgtcaccccatacaccgatccccccacgcatggccctctttgtccccagcctttggcacgcccgacctcattctctgctctggagtagggcaggtcaggaaagcgaaatacagcgccggcaaacagggtctgaatgccccgcgtggcattctttccgctccaggaccgccctgggcctgcaggatcctgggcgggagcccaggtgtccgggatctgggccactagggactggggaggaacctctcagagaagcccatagcccgcagcggccccgcgcggccggttccggcgccgcactgttccagcctctactatggtacagtccctgcgtcgcagcctcggcgggggctctaagaacgggaggcagaaaaagctcaatcagcagcaggcgagcttcacccgctgcttccaaatctgtgccaaaatattctatgctgcacagataaaatcctctgtcggttctacaagcctggcttttcctatagagaaccctcttataagcaaaaagtaaagctctcgtgaaga

SEQ ID NO: 90 - DNA, 851 bp, synthetic construct
  source 1..851: mol_type = other DNA
tttggttttccacactttgaaaactgatggatgctgccccattaaccatttcaggattttgttcgacaaataaaagagaatttagtttcagcagtccgcaaatcttagaattttggggacagagatggggacggggacagatatgggaggacggagacggcggggcggtggggtggtagcagaaatctttcacaaacaactagggagagctacaaagaacggtttcttagggtaggggatgtagctcagtggtagagcgcatgcttcgcatgtatgaggtcccgggttcgatccccggcatctccaagttgttttatcctatctgcccaatactattagaaactatatttcaaaatgtgaaataattgtgttttgtatcctccacttctttaactttttatggctttcttttagccactgtatgtagaattgtatatggagcacggggaacaagattgctttgaagtattttgaactatctatgtgaaacaaatggaattgagaacacagaaaaacatgcccaatggggagatgacagaagaggagcaacctggaaagttgcccggaacccattaatgagccctatttgtggtatatggtttgcctttagggcagaaataatcttggcttgagaacatggaaaggggagggaagacgggctattgttagtcctgtgagtcataggatgataaaaataggacaaatatcgaaagaaggatgaatctcagctcagcacgggcgaatttggctgctctagtgcaccggtctcccgtaggggaataacaaaaatgaaaacattccgaaacactgatgaaattcttgatgcagtttttaaaacttcttggagacag

SEQ ID NO: 91 - DNA, 55 bp, synthetic construct
  source 1..55: mol_type = other DNA
agcgctccggtttttctgtgctgaacctcaggggacgccgacacacgtacacgtc

SEQ ID NO: 92 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttgtgggttcgagtcccaccagagtcggaccttttttt

SEQ ID NO: 93 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttgtgggttcgagtcccaccagagtcgggtattttttt

SEQ ID NO: 94 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttgtgggttcgagtcccaccagagtcggtcattttttt

SEQ ID NO: 95 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttgtgggttcgagtcccaccagagtcgaggcttttttt

SEQ ID NO: 96 - DNA, 139 bp, synthetic construct; identical to SEQ ID NO: 40
  source 1..139: mol_type = other DNA

SEQ ID NO: 97 - DNA, 139 bp, synthetic construct; identical to SEQ ID NO: 92
  source 1..139: mol_type = other DNA

SEQ ID NO: 98 - DNA, 139 bp, synthetic construct; identical to SEQ ID NO: 93
  source 1..139: mol_type = other DNA

SEQ ID NO: 99 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttgtgggttcgagtcccaccagagtcgaagcttttttt

SEQ ID NO: 100 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttgtgggttcgagtcccaccagagtcgaaagttttttt

SEQ ID NO: 101 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttgtgggttcgagtcccaccagagtcgctttttttttt

SEQ ID NO: 102 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttgtgggttcgagtcccaccagagtcgaatgttttttt

SEQ ID NO: 103 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttgtgggttcgagtcccaccagagtcgctatttttttt

SEQ ID NO: 104 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttgtgggttcgagtcccaccagagtcgggatttttttt

SEQ ID NO: 105 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttgtgggttcgagtcccaccagagtcgagctttttttt

SEQ ID NO: 106 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttgtgggttcgagtcccaccagagtcgagacttttttt

SEQ ID NO: 107 - DNA, 139 bp, synthetic construct; identical to SEQ ID NO: 40
  source 1..139: mol_type = other DNA

SEQ ID NO: 108 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttgcgggttcgagtcccgccagagtcggtccttttttt

SEQ ID NO: 109 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttccgggttcgagtcccggcagagtcggtccttttttt

SEQ ID NO: 110 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttcccggttcgagtccgggcagagtcggtccttttttt

SEQ ID NO: 111 - DNA, 139 bp, synthetic construct
  source 1..139: mol_type = other DNA
agcgctccggtttttctgtgctgaacctcaggggacgccgacacacgtacacgtcggctctgtggcgcaatggatagcgcattggacttcaaattcaaaggttacgggttcgagtcccgtcagagtcggtccttttttt

SEQ ID NO: 112 - DNA, 139 bp, synthetic construct; identical to SEQ ID NO: 40
  source 1..139: mol_type = other DNA

SEQ ID NO: 113 - DNA, 149 bp, synthetic construct
  source 1..149: mol_type = other DNA
agcgctccggtttttctgtgctgaacctcaggggacgccgacacacgtacacgtcaccagaatggccgagtggttaaggcgttggacttcagatccaatggattcatatccgcccgggttcgaaccccggttctggtagtccttttttt

SEQ ID NO: 114 - DNA, 149 bp, synthetic construct
  source 1..149: mol_type = other DNA
agcgctccggtttttctgtgctgaacctcaggggacgccgacacacgtacacgtcaccagaatggccgagtggttaaggcgttggacttcagatccaatggattcatatccgcacgggttcgaaccccgtttctggtagtccttttttt

SEQ ID NO: 115 - DNA, 149 bp, synthetic construct
  source 1..149: mol_type = other DNA
agcgctccggtttttctgtgctgaacctcaggggacgccgacacacgtacacgtcaccagaatggccgagtggttaaggcgttggacttcagatccaatggattcatatccgcgcgggttcgaaccctgcttctggtagtccttttttt

SEQ ID NO: 116 - DNA, 149 bp, synthetic construct
  source 1..149: mol_type = other DNA
agcgctccggtttttctgtgctgaacctcaggggacgccgacacacgtacacgtcaccagaatggccgagtggttaaggcgttggacttcagatccaatggattcatatccgcgtgggttcgaaccccacttctggtagtccttttttt

SEQ ID NO: 117 - DNA, 138 bp, synthetic construct
  source 1..138: mol_type = other DNA
agcgctccggtttttctgtgctgaacctcaggggacgccgacacacgtacacgtcgcgttggtggtatagtggttagcatagctgccttcaaagcagttgacccgggttcgattcccggccaacgcagtccttttttt

SEQ ID NO: 118 - DNA, 139 bp, synthetic construct
  source 1..139: mol_type = other DNA
agcgctccggtttttctgtgctgaacctcaggggacgccgacacacgtacacgtcgcccctgtggcgcaatggatagcgcattggacttcaaatccaaaggttgtgggttcgagtcccaccaggggcggtccttttttt

SEQ ID NO: 119 - DNA, 139 bp, synthetic construct
  source 1..139: mol_type = other DNA
agcgctccggtttttctgtgctgaacctcaggggacgccgacacacgtacacgtcggccccgtggcgcaatggatagcgcattggacttcaaattcaaaggttgtgggttcgagtcccaccggggtcggtccttttttt

SEQ ID NO: 120 - DNA, 139 bp, synthetic construct
  source 1..139: mol_type = other DNA
agcgctccggtttttctgtgctgaacctcaggggacgccgacacacgtacacgtcgcccctgtggcgcaatggatagcgcattggacttcaaattcaaaggttgtgggttcgagtcccaccaggggcggtccttttttt

SEQ ID NO: 121 - DNA, 139 bp, synthetic construct
  source 1..139: mol_type = other DNA
agcgctccggtttttctgtgctgaacctcaggggacgccgacacacgtacacgtcggccctgtggcgcaatggatagcgcatcggacttcaaattcgaaggttgtgggttcgagtcccaccagggtcggtccttttttt

SEQ ID NO: 122 - DNA, 139 bp, synthetic construct
  source 1..139: mol_type = other DNA
agcgctccggtttttctgtgctgaacctcaggggacgccgacacacgtacacgtcggccctgtggcgcaatggatagcgcattggacttcaaattcaaaggttgtgggttcgagtcccaccagggtcggtccttttttt

SEQ ID NO: 123 - DNA, 139 bp, synthetic construct
  source 1..139: mol_type = other DNA
agcgctccggtttttctgtgctgaacctcaggggacgccgacacacgtacacgtcgcctccgtggcgcaatggatagcgcatcggacttcaaattcgaaggttgtgggttcgagtcccaccggaggcggtccttttttt

SEQ ID NO: 124 - DNA, 139 bp, synthetic construct
  source 1..139: mol_type = other DNA
agcgctccggtttttctgtgctgaacctcaggggacgccgacacacgtacacgtcgcccccgtggcgcaatggatagcgcatcggacttcaaattcgaaggttgtgggttcgagtcccaccgggggcggtccttttttt

SEQ ID NO: 125 - DNA, 139 bp, synthetic construct
  source 1..139: mol_type = other DNA
agcgctccggtttttctgtgctgaacctcaggggacgccgacacacgtacacgtcgcccctgtggcgcaatggatagcgcatcggccttcaaagccgaaggttgtgggttcgagtcccaccaggggcggtccttttttt

SEQ ID NO: 126 - DNA, 139 bp, synthetic construct
  source 1..139: mol_type = other DNA
agcgctccggtttttctgtgctgaacctcaggggacgccgacacacgtacacgtcgcccctgtggcgcaatggatagcgcatcggacttcaaatccgaaggttgtgggttcgagtcccaccaggggcggtccttttttt

SEQ ID NO: 127 - DNA, 139 bp, synthetic construct
  source 1..139: mol_type = other DNA
agcgctccggtttttctgtgctgaacctcaggggacgccgacacacgtacacgtcgcccccgtggcgcaatggatagcgcattggacttcaaatccaaaggttgtgggttcgagtcccaccgggggcggtccttttttt

SEQ ID NO: 128 - DNA, 139 bp, synthetic construct
  source 1..139: mol_type = other DNA
agcgctccggtttttctgtgctgaacctcaggggacgccgacacacgtacacgtcggccccgtggcgcaatggatagcgcaccggacttcaaatccggaggttgtgggttcgagtcccaccggggtcggtccttttttt

SEQ ID NO: 129 - DNA, 139 bp, synthetic construct
  source 1..139: mol_type = other DNA
agcgctccggtttttctgtgctgaacctcaggggacgccgacacacgtacacgtcgcccctgtggcgcaatggatagcgcaccggacttcaaattcggaggttgtgggttcgagtcccaccaggggcggtccttttttt

SEQ ID NO: 130 - DNA, 139 bp, synthetic construct
  source 1..139: mol_type = other DNA
agcgctccggtttttctgtgctgaacctcaggggacgccgacacacgtacacgtcggccctgtggcgcaatggatagcgcattggccttcaaagccaaaggttgtgggttcgagtcccaccagggtcggtccttttttt

SEQ ID NO: 131 - DNA, 139 bp, synthetic construct
  source 1..139: mol_type = other DNA
agcgctccggtttttctgtgctgaacctcaggggacgccgacacacgtacacgtcgcctccgtggcgcaatggatagcgcattggacttcaaatccaaaggttgtgggttcgagtcccaccggaggcggtccttttttt

SEQ ID NO: 132 - DNA, 139 bp, synthetic construct; identical to SEQ ID NO: 40
  source 1..139: mol_type = other DNA

SEQ ID NO: 133 - DNA, 149 bp, synthetic construct
  source 1..149: mol_type = other DNA
agcgctccggtttttctgtgctgaacctcaggggacgccgacacacgtacacgtcgccaggatggccgagtggttaaggcgccggacttcagatccggtggactcatgtccgcgtgggttcgaaccccactcctggcagtccttttttt

SEQ ID NO: 134 - DNA, 149 bp, synthetic construct
  source 1..149: mol_type = other DNA
agcgctccggtttttctgtgctgaacctcaggggacgccgacacacgtacacgtcgccaggatggccgagtggttaaggcgccggacttcagatccggtggattcatatccgcgtgggttcgaaccccactcctggcagtccttttttt

SEQ ID NO: 135 - DNA, 149 bp, synthetic construct
  source 1..149: mol_type = other DNA
agcgctccggtttttctgtgctgaacctcaggggacgccgacacacgtacacgtcgccaggatggccgagtggttaaggcgctggacttcagatccagtggattcatatccgcgtgggttcgaaccccactcctggcagtccttttttt

SEQ ID NO: 136 - DNA, 149 bp, synthetic construct
  source 1..149: mol_type = other DNA
agcgctccggtttttctgtgctgaacctcaggggacgccgacacacgtacacgtcgccgggatggccgagtggttaaggcgtcggacttcagatccgatggattcatatccgcgtgggttcgaaccccactcccggcagtccttttttt

SEQ ID NO: 137 - DNA, 149 bp, synthetic construct
  source 1..149: mol_type = other DNA
agcgctccggtttttctgtgctgaacctcaggggacgccgacacacgtacacgtcgccaggatggccgagtggttaaggcgtcggacttcagatccgatggactcatgtccgcgtgggttcgaaccccactcctggcagtccttttttt

SEQ ID NO: 138 - DNA, 149 bp, synthetic construct
  source 1..149: mol_type = other DNA
agcgctccggtttttctgtgctgaacctcaggggacgccgacacacgtacacgtcgccaggatggccgagtggttaaggcgttggacttcagatccaatggattcatatccgcgtgggttcgaaccccactcctggcagtccttttttt

SEQ ID NO: 139 - DNA, 149 bp, synthetic construct
  source 1..149: mol_type = other DNA
agcgctccggtttttctgtgctgaacctcaggggacgccgacacacgtacacgtcgccaggatggccgagtggttaaggcgttggacttcagatccaatggactcatgtccgcgtgggttcgaaccccactcctggcagtccttttttt

SEQ ID NO: 140 - DNA, 149 bp, synthetic construct
  source 1..149: mol_type = other DNA
agcgctccggtttttctgtgctgaacctcaggggacgccgacacacgtacacgtcgccgggatggccgagtggttaaggcgctggacttcagatccagtggattcatatccgcgtgggttcgaaccccactcccggcagtccttttttt

SEQ ID NO: 141 - DNA, 149 bp, synthetic construct
  source 1..149: mol_type = other DNA
agcgctccggtttttctgtgctgaacctcaggggacgccgacacacgtacacgtcgccaggatggccgagtggttaaggcgtcggacttcagatccgatggcttcatagccgcgtgggttcgaaccccactcctggcagtccttttttt

SEQ ID NO: 142 - DNA, 149 bp, synthetic construct
  source 1..149: mol_type = other DNA
agcgctccggtttttctgtgctgaacctcaggggacgccgacacacgtacacgtcgccgggatggccgagtggttaaggcgccggacttcagatccggtggattcatatccgcgtgggttcgaaccccactcccggcagtccttttttt

SEQ ID NO: 143 - DNA, 149 bp, synthetic construct
  source 1..149: mol_type = other DNA
agcgctccggtttttctgtgctgaacctcaggggacgccgacacacgtacacgtcaccagaatggccgagtggttaaggcgttggacttcagatccaatggactcatgtccgcgtgggttcgaaccccacttctggtagtccttttttt

SEQ ID NO: 144 - DNA, 149 bp, synthetic construct
  source 1..149: mol_type = other DNA
agcgctccggtttttctgtgctgaacctcaggggacgccgacacacgtacacgtcgccgggatggccgagtggttaaggcgtcggacttcagatccgatggactcatgtccgcgtgggttcgaaccccactcccggcagtccttttttt

SEQ ID NO: 145 - DNA, 149 bp, synthetic construct
  source 1..149: mol_type = other DNA
agcgctccggtttttctgtgctgaacctcaggggacgccgacacacgtacacgtcgccaggatggccgagtggttaaggcgctggacttcagatccagtggactcatgtccgcgtgggttcgaaccccactcctggcagtccttttttt

SEQ ID NO: 146 - DNA, 149 bp, synthetic construct
  source 1..149: mol_type = other DNA
agcgctccggtttttctgtgctgaacctcaggggacgccgacacacgtacacgtcgccaggatggccgagtggttaaggcgttggacttcagatccaatggcttcatagccgcgtgggttcgaaccccactcctggcagtccttttttt

SEQ ID NO: 147 - DNA, 149 bp, synthetic construct; identical to SEQ ID NO: 116
  source 1..149: mol_type = other DNA

SEQ ID NO: 148 - DNA, 55 bp, synthetic construct; identical to SEQ ID NO: 91
  source 1..55: mol_type = other DNA

SEQ ID NO: 149 - DNA, 248 bp, synthetic construct
  source 1..248: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacac

SEQ ID NO: 150 - DNA, 222 bp, synthetic construct
  source 1..222: mol_type = other DNA
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacgaaacac

SEQ ID NO: 151 - DNA, 55 bp, synthetic construct
  source 1..55: mol_type = other DNA
gccaggggtgtggccatacaggtttatagtggttagtagagacaagtaagtgggt

SEQ ID NO: 152 - DNA, 139 bp, synthetic construct
  source 1..139: mol_type = other DNA
gccaggggtgtggccatacaggtttatagtggttagtagagacaagtaagtgggtggctctgtggcgcaatggatagcgcattggacttcaaattcaaaggttgtgggttcgagtcccaccagagtcggaccttttttt

SEQ ID NO: 153 - DNA, 139 bp, synthetic construct; identical to SEQ ID NO: 40
  source 1..139: mol_type = other DNA

SEQ ID NO: 154 - DNA, 139 bp, synthetic construct
  source 1..139: mol_type = other DNA
gccaggggtgtggccatacaggtttatagtggttagtagagacaagtaagtgggtggctctgtggcgcaatggatagcgcattggacttcaaattcaaaggttgcgggttcgagtcccgccagagtcggaccttttttt

SEQ ID NO: 155 - DNA, 139 bp, synthetic construct
  source 1..139: mol_type = other DNA
gccaggggtgtggccatacaggtttatagtggttagtagagacaagtaagtgggtgcccctgtggcgcaatggatagcgcattggacttcaaatccaaaggttgtgggttcgagtcccaccaggggcggaccttttttt

SEQ ID NO: 156 - DNA, 139 bp, synthetic construct
  source 1..139: mol_type = other DNA
gccaggggtgtggccatacaggtttatagtggttagtagagacaagtaagtgggtgcccctgtggcgcaatggatagcgcattggacttcaaatccaaaggttgcgggttcgagtcccgccaggggcggaccttttttt

SEQ ID NO: 157 - DNA, 163 bp, synthetic construct
  source 1..163: mol_type = other DNA
gccaggggtgtggccatacaggtttatagtggttagtagagacaagtaagtgggtgcccctgtggcgcaatggatagcgcattggacttcaaatccaaaggttgcgggttcgagtcccgccaggggcgctttttttccccatcagtttttataaacttacaca

SEQ ID NO: 158 - DNA, 163 bp, synthetic construct
  source 1..163: mol_type = other DNA
gccaggggtgtggccatacaggtttatagtggttagtagagacaagtaagtgggtgcccctgtggcgcaatggatagcgcattggacttcaaatccaaaggttgcgggttcgagtcccgccaggggcggttccatttttaaaagtgtctcttctgggcctgaa

SEQ ID NO: 159 - DNA, 167 bp, synthetic construct
  source 1..167: mol_type = other DNA
gccaggggtgtggccatacaggtttatagtggttagtagagacaagtaagtgggtgcccctgtggcgcaatggatagcgcattggacttcaaatccaaaggttgcgggttcgagtcccgccaggggcggaccctttttttccccatcagtttttataaacttacaca

SEQ ID NO: 160 - DNA, 167 bp, synthetic construct
  source 1..167: mol_type = other DNA
gccaggggtgtggccatacaggtttatagtggttagtagagacaagtaagtgggtgcccctgtggcgcaatggatagcgcattggacttcaaatccaaaggttgcgggttcgagtcccgccaggggcggaccgttccatttttaaaagtgtctcttctgggcctgaa

SEQ ID NO: 161 - DNA, 163 bp, synthetic construct
  source 1..163: mol_type = other DNA
gccaggggtgtggccatacaggtttatagtggttagtagagacaagtaagtgggtgcccctgtggcgcaatggatagcgcattggacttcaaatccaaaggttgcgggttcgagtcccgccaggggcggaccttttccccatcagtttttataaacttacaca

SEQ ID NO: 162 - DNA, 163 bp, synthetic construct
  source 1..163: mol_type = other DNA
gccaggggtgtggccatacaggtttatagtggttagtagagacaagtaagtgggtgcccctgtggcgcaatggatagcgcattggacttcaaatccaaaggttgcgggttcgagtcccgccaggggcggacccatttttaaaagtgtctcttctgggcctgaa

SEQ ID NO: 163 - DNA, 138 bp, synthetic construct; identical to SEQ ID NO: 117
  source 1..138: mol_type = other DNA

SEQ ID NO: 164 - DNA, 138 bp, synthetic construct
  source 1..138: mol_type = other DNA
tgtcctgagttaagcgaccaatagggaccatctgggagagctgctgagagtaggtgcgttggtggtatagtggttagcatagctgccttcaaagcagttgacccgggttcgattcccggccaacgcagtccttttttt

SEQ ID NO: 165 - DNA, 138 bp, synthetic construct
  source 1..138: mol_type = other DNA
gccaggggtgtggccatacaggtttatagtggttagtagagacaagtaagtgggtgcgttggtggtatagtggttagcatagctgccttcaaagcagttgacccgggttcgattcccggccaacgcagtccttttttt

SEQ ID NO: 166 - DNA, 138 bp, synthetic construct
  source 1..138: mol_type = other DNA
tcaatctgtaagagcatcttctcttatacctttccaaatccttattatttctgtggcgttggtggtatagtggttagcatagctgccttcaaagcagttgacccgggttcgattcccggccaacgcagtccttttttt

SEQ ID NO: 167 - DNA, 138 bp, synthetic construct
  source 1..138: mol_type = other DNA
atagcgtttagacaagagacagaagtgagtcctgagcacaatgtagcataagcgcgcgttggtggtatagtggttagcatagctgccttcaaagcagttgacccgggttcgattcccggccaacgcagtccttttttt

SEQ ID NO: 168 - DNA, 138 bp, synthetic construct
  source 1..138: mol_type = other DNA
gatgaggttgcggtaagcattagagggctatcagcagcatcttatcgcagcggaggcgttggtggtatagtggttagcatagctgccttcaaagcagttgacccgggttcgattcccggccaacgcagtccttttttt

SEQ ID NO: 169 - DNA, 138 bp, synthetic construct
  source 1..138: mol_type = other DNA
agcgctccggtttttctgtgctgaacctcaggggacgccgacacacgtacacgtcgcgttggtggtatagtggttagcatagctgccttcaaagcagttgacccgggttcgattcccggccaacgcagaccttttttt

SEQ ID NO: 170 - DNA, 138 bp, synthetic construct
  source 1..138: mol_type = other DNA
gccaggggtgtggccatacaggtttatagtggttagtagagacaagtaagtgggtgcgttggtggtatagtggttagcatagctgccttcaaagcagttgacccgggttcgattcccggccaacgcagaccttttttt

SEQ ID NO: 171 - DNA, 138 bp, synthetic construct
  source 1..138: mol_type = other DNA
gccaggggtgtggccatacaggtttatagtggttagtagagacaagtaagtgggtgcgttggtggtatagtggttagcatagctgccttcaaagcagttgaccccggttcgattccgggccaacgcagaccttttttt

SEQ ID NO: 172 - DNA, 162 bp, synthetic construct
  source 1..162: mol_type = other DNA
gccaggggtgtggccatacaggtttatagtggttagtagagacaagtaagtgggtgcgttggtggtatagtggttagcatagctgccttcaaagcagttgaccccggttcgattccgggccaacgcactttttttccccatcagtttttataaacttacaca

SEQ ID NO: 173 - DNA, 162 bp, synthetic construct
  source 1..162: mol_type = other DNA
gccaggggtgtggccatacaggtttatagtggttagtagagacaagtaagtgggtgcgttggtggtatagtggttagcatagctgccttcaaagcagttgaccccggttcgattccgggccaacgcagttccatttttaaaagtgtctcttctgggcctgaa

SEQ ID NO: 174 - DNA, 166 bp, synthetic construct
  source 1..166: mol_type = other DNA
gccaggggtgtggccatacaggtttatagtggttagtagagacaagtaagtgggtgcgttggtggtatagtggttagcatagctgccttcaaagcagttgaccccggttcgattccgggccaacgcagaccctttttttccccatcagtttttataaacttacaca

SEQ ID NO: 175 - DNA, 166 bp, synthetic construct
  source 1..166: mol_type = other DNA
gccaggggtgtggccatacaggtttatagtggttagtagagacaagtaagtgggtgcgttggtggtatagtggttagcatagctgccttcaaagcagttgaccccggttcgattccgggccaacgcagaccgttccatttttaaaagtgtctcttctgggcctgaa

SEQ ID NO: 176 - DNA, 162 bp, synthetic construct
  source 1..162: mol_type = other DNA
gccaggggtgtggccatacaggtttatagtggttagtagagacaagtaagtgggtgcgttggtggtatagtggttagcatagctgccttcaaagcagttgaccccggttcgattccgggccaacgcagaccttttccccatcagtttttataaacttacaca

SEQ ID NO: 177 - DNA, 162 bp, synthetic construct
  source 1..162: mol_type = other DNA
gccaggggtgtggccatacaggtttatagtggttagtagagacaagtaagtgggtgcgttggtggtatagtggttagcatagctgccttcaaagcagttgaccccggttcgattccgggccaacgcagacccatttttaaaagtgtctcttctgggcctgaa

SEQ ID NO: 178 - DNA, 149 bp, synthetic construct; identical to SEQ ID NO: 116
  source 1..149: mol_type = other DNA

SEQ ID NO: 179 - DNA, 149 bp, synthetic construct
  source 1..149: mol_type = other DNA
tgtcctgagttaagcgaccaatagggaccatctgggagagctgctgagagtaggtaccagaatggccgagtggttaaggcgttggacttcagatccaatggattcatatccgcgtgggttcgaaccccacttctggtagtccttttttt

SEQ ID NO: 180 - DNA, 149 bp, synthetic construct
  source 1..149: mol_type = other DNA
gccaggggtgtggccatacaggtttatagtggttagtagagacaagtaagtgggtaccagaatggccgagtggttaaggcgttggacttcagatccaatggattcatatccgcgtgggttcgaaccccacttctggtagtccttttttt

SEQ ID NO: 181 - DNA, 149 bp, synthetic construct
  source 1..149: mol_type = other DNA
tcaatctgtaagagcatcttctcttatacctttccaaatccttattatttctgtgaccagaatggccgagtggttaaggcgttggacttcagatccaatggattcatatccgcgtgggttcgaaccccacttctggtagtccttttttt

SEQ ID NO: 182 - DNA, 149 bp, synthetic construct
  source 1..149: mol_type = other DNA
atagcgtttagacaagagacagaagtgagtcctgagcacaatgtagcataagcgcaccagaatggccgagtggttaaggcgttggacttcagatccaatggattcatatccgcgtgggttcgaaccccacttctggtagtccttttttt

SEQ ID NO: 183 - DNA, 149 bp, synthetic construct
  source 1..149: mol_type = other DNA
gatgaggttgcggtaagcattagagggctatcagcagcatcttatcgcagcggagaccagaatggccgagtggttaaggcgttggacttcagatccaatggattcatatccgcgtgggttcgaaccccacttctggtagtccttttttt

SEQ ID NO: 184 - DNA, 149 bp, synthetic construct
  source 1..149: mol_type = other DNA
agcgctccggtttttctgtgctgaacctcaggggacgccgacacacgtacacgtcaccagaatggccgagtggttaaggcgttggacttcagatccaatggattcatatccgcgtgggttcgaaccccacttctggtagaccttttttt

SEQ ID NO: 185 - DNA, 149 bp, synthetic construct
  source 1..149: mol_type = other DNA
gccaggggtgtggccatacaggtttatagtggttagtagagacaagtaagtgggtaccagaatggccgagtggttaaggcgttggacttcagatccaatggattcatatccgcgtgggttcgaaccccacttctggtagaccttttttt

SEQ ID NO: 186 - DNA, 149 bp, synthetic construct
  source 1..149: mol_type = other DNA
gccaggggtgtggccatacaggtttatagtggttagtagagacaagtaagtgggtaccagaatggccgagtggttaaggcgttggacttcagatccaatggattcatatccgcccgggttcgaaccccggttctggtagaccttttttt

SEQ ID NO: 187 - DNA, 149 bp, synthetic construct
  source 1..149: mol_type = other DNA
gccaggggtgtggccatacaggtttatagtggttagtagagacaagtaagtgggtgccaggatggccgagtggttaaggcgccggacttcagatccggtggactcatgtccgcgtgggttcgaaccccactcctggcagaccttttttt

SEQ ID NO: 188 - DNA, 149 bp, synthetic construct
  source 1..149: mol_type = other DNA
gccaggggtgtggccatacaggtttatagtggttagtagagacaagtaagtgggtgccaggatggccgagtggttaaggcgccggacttcagatccggtggactcatgtccgcccgggttcgaaccccggtcctggcagaccttttttt

SEQ ID NO: 189 - DNA, 173 bp, synthetic construct
  source 1..173: mol_type = other DNA
gccaggggtgtggccatacaggtttatagtggttagtagagacaagtaagtgggtgccaggatggccgagtggttaaggcgccggacttcagatccggtggactcatgtccgcccgggttcgaaccccggtcctggcactttttttccccatcagtttttataaacttacaca

SEQ ID NO: 190 - DNA, 173 bp, synthetic construct
  source 1..173: mol_type = other DNA
gccaggggtgtggccatacaggtttatagtggttagtagagacaagtaagtgggtgccaggatggccgagtggttaaggcgccggacttcagatccggtggactcatgtccgcccgggttcgaaccccggtcctggcagttccatttttaaaagtgtctcttctgggcctgaa

SEQ ID NO: 191 - DNA, 177 bp, synthetic construct
  source 1..177: mol_type = other DNA
gccaggggtgtggccatacaggtttatagtggttagtagagacaagtaagtgggtgccaggatggccgagtggttaaggcgccggacttcagatccggtggactcatgtccgcccgggttcgaaccccggtcctggcagaccctttttttccccatcagtttttataaacttacaca

SEQ ID NO: 192 - DNA, 177 bp, synthetic construct
  source 1..177: mol_type = other DNA
gccaggggtgtggccatacaggtttatagtggttagtagagacaagtaagtgggtgccaggatggccgagtggttaaggcgccggacttcagatccggtggactcatgtccgcccgggttcgaaccccggtcctggcagaccgttccatttttaaaagtgtctcttctgggcctgaa

SEQ ID NO: 193 - DNA, 173 bp, synthetic construct
  source 1..173: mol_type = other DNA
gccaggggtgtggccatacaggtttatagtggttagtagagacaagtaagtgggtgccaggatggccgagtggttaaggcgccggacttcagatccggtggactcatgtccgcccgggttcgaaccccggtcctggcagaccttttccccatcagtttttataaacttacaca

SEQ ID NO: 194 - DNA, 173 bp, synthetic construct
  source 1..173: mol_type = other DNA
gccaggggtgtggccatacaggtttatagtggttagtagagacaagtaagtgggtgccaggatggccgagtggttaaggcgccggacttcagatccggtggactcatgtccgcccgggttcgaaccccggtcctggcagacccatttttaaaagtgtctcttctgggcctgaa

SEQ ID NO: 195 - DNA, 138 bp, synthetic construct
  source 1..138: mol_type = other DNA
agcgctccggtttttctgtgctgaacctcaggggacgccgacacacgtacacgtcggcctcgtggcgcaacggtagcgcgtctgacttcagatcagaaggttgcgtgttcaaatcacgtcggggtcagtccttttttt

SEQ ID NO: 196 - DNA, 138 bp, synthetic construct
  source 1..138: mol_type = other DNA
tgtcctgagttaagcgaccaatagggaccatctgggagagctgctgagagtaggtggcctcgtggcgcaacggtagcgcgtctgacttcagatcagaaggttgcgtgttcaaatcacgtcggggtcagtccttttttt

SEQ ID NO: 197 - DNA, 138 bp, synthetic construct
  source 1..138: mol_type = other DNA
gccaggggtgtggccatacaggtttatagtggttagtagagacaagtaagtgggtggcctcgtggcgcaacggtagcgcgtctgacttcagatcagaaggttgcgtgttcaaatcacgtcggggtcagtccttttttt

SEQ ID NO: 198 - DNA, 138 bp, synthetic construct
  source 1..138: mol_type = other DNA
tcaatctgtaagagcatcttctcttatacctttccaaatccttattatttctgtgggcctcgtggcgcaacggtagcgcgtctgacttcagatcagaaggttgcgtgttcaaatcacgtcggggtcagtccttttttt

SEQ ID NO: 199 - DNA, 138 bp, synthetic construct
  source 1..138: mol_type = other DNA
atagcgtttagacaagagacagaagtgagtcctgagcacaatgtagcataagcgcggcctcgtggcgcaacggtagcgcgtctgacttcagatcagaaggttgcgtgttcaaatcacgtcggggtcagtccttttttt

SEQ ID NO: 200 - DNA, 138 bp, synthetic construct
  source 1..138: mol_type = other DNA
gatgaggttgcggtaagcattagagggctatcagcagcatcttatcgcagcggagggcctcgtggcgcaacggtagcgcgtctgacttcagatcagaaggttgcgtgttcaaatcacgtcggggtcagtccttttttt

SEQ ID NO: 201 - DNA, 138 bp, synthetic construct
  source 1..138: mol_type = other DNA
agcgctccggtttttctgtgctgaacctcaggggacgccgacacacgtacacgtcggcctcgtggcgcaacggtagcgcgtctgacttcagatcagaaggttgcgtgttcaaatcacgtcggggtcagaccttttttt

SEQ ID NO: 202 - DNA, 138 bp, synthetic construct
  source 1..138: mol_type = other DNA
gccaggggtgtggccatacaggtttatagtggttagtagagacaagtaagtgggtggcctcgtggcgcaacggtagcgcgtctgacttcagatcagaaggttgcgtgttcaaatcacgtcggggtcagaccttttttt

SEQ ID NO: 203 - DNA, 138 bp, synthetic construct
  source 1..138: mol_type = other DNA
agcgctccggtttttctgtgctgaacctcaggggacgccgacacacgtacacgtcggcctcgtggcgcaacggtagcgcgtctgacttcagatcagaaggttccgggttcaaatcccggcggggtcagtccttttttt

SEQ ID NO: 204 - DNA, 138 bp, synthetic construct
  source 1..138: mol_type = other DNA
gccaggggtgtggccatacaggtttatagtggttagtagagacaagtaagtgggtggcctcgtggcgcaacggtagcgcgtctgacttcagatcagaaggttccgggttcaaatcccggcggggtcagaccttttttt

SEQ ID NO: 205 - DNA, 162 bp, synthetic construct
  source 1..162: mol_type = other DNA
gccaggggtgtggccatacaggtttatagtggttagtagagacaagtaagtgggtggcctcgtggcgcaacggtagcgcgtctgacttcagatcagaaggttccgggttcaaatcccggcggggtcactttttttccccatcagtttttataaacttacaca

SEQ ID NO: 206 - DNA, 162 bp, synthetic construct
  source 1..162: mol_type = other DNA
gccaggggtgtggccatacaggtttatagtggttagtagagacaagtaagtgggtggcctcgtggcgcaacggtagcgcgtctgacttcagatcagaaggttccgggttcaaatcccggcggggtcagttccatttttaaaagtgtctcttctgggcctgaa

SEQ ID NO: 207 - DNA, 166 bp, synthetic construct
  source 1..166: mol_type = other DNA
gccaggggtgtggccatacaggtttatagtggttagtagagacaagtaagtgggtggcctcgtggcgcaacggtagcgcgtctgacttcagatcagaaggttccgggttcaaatcccggcggggtcagaccctttttttccccatcagtttttataaacttacaca

SEQ ID NO: 208 - DNA, 166 bp, synthetic construct
  source 1..166: mol_type = other DNA
gccaggggtgtggccatacaggtttatagtggttagtagagacaagtaagtgggtggcctcgtggcgcaacggtagcgcgtctgacttcagatcagaaggttccgggttcaaatcccggcggggtcagaccgttccatttttaaaagtgtctcttctgggcctgaa

SEQ ID NO: 209 - DNA, 162 bp, synthetic construct
  source 1..162: mol_type = other DNA
gccaggggtgtggccatacaggtttatagtggttagtagagacaagtaagtgggtggcctcgtggcgcaacggtagcgcgtctgacttcagatcagaaggttccgggttcaaatcccggcggggtcagaccttttccccatcagtttttataaacttacaca

SEQ ID NO: 210 - DNA, 162 bp, synthetic construct
  source 1..162: mol_type = other DNA
gccaggggtgtggccatacaggtttatagtggttagtagagacaagtaagtgggtggcctcgtggcgcaacggtagcgcgtctgacttcagatcagaaggttccgggttcaaatcccggcggggtcagacccatttttaaaagtgtctcttctgggcctgaa

SEQ ID NO: 211 - DNA, 138 bp, synthetic construct; identical to SEQ ID NO: 117
  source 1..138: mol_type = other DNA

SEQ ID NO: 212 - DNA, 138 bp, synthetic construct; identical to SEQ ID NO: 195
  source 1..138: mol_type = other DNA

SEQ ID NO: 213 - DNA, 138 bp, synthetic construct
  source 1..138: mol_type = other DNA
agcgctccggtttttctgtgctgaacctcaggggacgccgacacacgtacacgtcgacctcgtggcgcaatggtagcgcgtctgactctagatcagaaggttgcgtgttcaagtcacgtcggggtcagtccttttttt

SEQ ID NO: 214 - DNA, 139 bp, synthetic construct; identical to SEQ ID NO: 40
  source 1..139: mol_type = other DNA

SEQ ID NO: 215 - DNA, 138 bp, synthetic construct
  source 1..138: mol_type = other DNA
agcgctccggtttttctgtgctgaacctcaggggacgccgacacacgtacacgtcggttccatggtgtaatggtgagcactctggactctaaatccagcgatccgagttcgagtctcggtggaacctgtccttttttt

SEQ ID NO: 216 - DNA, 138 bp, synthetic construct
  source 1..138: mol_type = other DNA
agcgctccggtttttctgtgctgaacctcaggggacgccgacacacgtacacgtcggttccatggtgtaatggtaagcactctggactttaaatccagcgatccgagttcgagtctcggtggaacctgtccttttttt

SEQ ID NO: 217 - DNA, 138 bp, synthetic construct
  source 1..138: mol_type = other DNA
agcgctccggtttttctgtgctgaacctcaggggacgccgacacacgtacacgtctcccacatggtctagcggttaggattcctggttctaacccaggcggcccgggttcgactcccggtgtgggaagtccttttttt

SEQ ID NO: 218 - DNA, 138 bp, synthetic construct
  source 1..138: mol_type = other DNA
agcgctccggtttttctgtgctgaacctcaggggacgccgacacacgtacacgtctcccacatggtctagcggttaggattcctggttttaacccaggcggcccgggttcgactcccggtgtgggaagtccttttttt

SEQ ID NO: 219 - DNA, 139 bp, synthetic construct
  source 1..139: mol_type = other DNA
agcgctccggtttttctgtgctgaacctcaggggacgccgacacacgtacacgtcccttcgatagctcagctggtagagcggaggactttagatccttaggtcgctggttcgattccggctcgaaggagtccttttttt

SEQ ID NO: 220 - DNA, 139 bp, synthetic construct
  source 1..139: mol_type = other DNA
agcgctccggtttttctgtgctgaacctcaggggacgccgacacacgtacacgtcccttcgatagctcagctggtagagcggaggactctagatccttaggtcgctggttcgattccggctcgaaggagtccttttttt

SEQ ID NO: 221 - DNA, 148 bp, synthetic construct
  source 1..148: mol_type = other DNA
agcgctccggtttttctgtgctgaacctcaggggacgccgacacacgtacacgtcggtagcgtggccgagcggtctaaggcgctggattctagctccagtctcttcgggggcgtgggttcaaatcccaccgctgccagtccttttttt

SEQ ID NO: 222 - DNA, 149 bp, synthetic construct; identical to SEQ ID NO: 116
  source 1..149: mol_type = other DNA

SEQ ID NO: 223 - DNA, 148 bp, synthetic construct
  source 1..148: mol_type = other DNA
agcgctccggtttttctgtgctgaacctcaggggacgccgacacacgtacacgtcgacgaggtggccgagtggttaaggcgatggactctaaatccattgtgctctgcacacgtgggttcgaatcccatcctcgtcggtccttttttt

SEQ ID NO: 224 - DNA, 148 bp, synthetic construct
  source 1..148: mol_type = other DNA
agcgctccggtttttctgtgctgaacctcaggggacgccgacacacgtacacgtcgtcacggtggccgagtggttaaggcgttggacttcaaatccaatggggtttccccgcacaggttcgaatcctgttcgtgacggtccttttttt

SEQ ID NO: 225 - DNA, 148 bp, synthetic construct
  source 1..148: mol_type = other DNA
agcgctccggtttttctgtgctgaacctcaggggacgccgacacacgtacacgtcgtagtcgtggccgagtggttaaggcgatggactttaaatccattggggtttccccacgcaggttcgaatcctgccgactacggtccttttttt

SEQ ID NO: 226 - DNA, 139 bp, synthetic construct
  source 1..139: mol_type = other DNA
agcgctccggtttttctgtgctgaacctcaggggacgccgacacacgtacacgtcgcctgggtagctcagtcggtagagcatcagactttaaatctgagggtccagggttcaagtccctgtccaggcggtccttttttt

SEQ ID NO: 227 - DNA, 139 bp, synthetic construct
  source 1..139: mol_type = other DNA
agcgctccggtttttctgtgctgaacctcaggggacgccgacacacgtacacgtcgggggtatagctcagtgggtagagcatttgacttcagatcaagaggtccccggttcaaatccgggtgccccctgtccttttttt

SEQ ID NO: 228 - DNA, 162 bp, synthetic construct
  source 1..162: mol_type = other DNA
gccaggggtgtggccatacaggtttatagtggttagtagagacaagtaagtgggtgcgttggtggtatagtggttagcatagctgccttcaaagcagttgacccgggttcgattcccggccaacgcagaccttttccccatcagtttttataaacttacaca

SEQ ID NO: 229 - DNA, 162 bp, synthetic construct
  source 1..162: mol_type = other DNA
gccaggggtgtggccatacaggtttatagtggttagtagagacaagtaagtgggtggcctcgtggcgcaacggtagcgcgtctgacttcagatcagaaggttgcgtgttcaaatcacgtcggggtcagaccttttccccatcagtttttataaacttacaca

SEQ ID NO: 230 - DNA, 162 bp, synthetic construct
  source 1..162: mol_type = other DNA
gccaggggtgtggccatacaggtttatagtggttagtagagacaagtaagtgggtgacctcgtggcgcaatggtagcgcgtctgactctagatcagaaggttgcgtgttcaagtcacgtcggggtcagaccttttccccatcagtttttataaacttacaca

SEQ ID NO: 231 - DNA, 163 bp, synthetic construct
  source 1..163: mol_type = other DNA
gccaggggtgtggccatacaggtttatagtggttagtagagacaagtaagtgggtggctctgtggcgcaatggatagcgcattggacttcaaattcaaaggttgtgggttcgagtcccaccagagtcggaccttttccccatcagtttttataaacttacaca

SEQ ID NO: 232 - DNA, 162 bp, synthetic construct
  source 1..162: mol_type = other DNA
gccaggggtgtggccatacaggtttatagtggttagtagagacaagtaagtgggtggttccatggtgtaatggtgagcactctggactctaaatccagcgatccgagttcgagtctcggtggaacctgaccttttccccatcagtttttataaacttacaca

SEQ ID NO: 233 - DNA, 162 bp, synthetic construct
  source 1..162: mol_type = other DNA
gccaggggtgtggccatacaggtttatagtggttagtagagacaagtaagtgggtggttccatggtgtaatggtaagcactctggactttaaatccagcgatccgagttcgagtctcggtggaacctgaccttttccccatcagtttttataaacttacaca

SEQ ID NO: 234 - DNA, 172 bp, synthetic construct
  source 1..172: mol_type = other DNA
gccaggggtgtggccatacaggtttatagtggttagtagagacaagtaagtgggtggtagcgtggccgagcggtctaaggcgctggattctagctccagtctcttcgggggcgtgggttcaaatcccaccgctgccagaccttttccccatcagtttttataaacttacaca

SEQ ID NO: 235 - DNA, 173 bp, synthetic construct
  source 1..173: mol_type = other DNA
gccaggggtgtggccatacaggtttatagtggttagtagagacaagtaagtgggtaccagaatggccgagtggttaaggcgttggacttcagatccaatggattcatatccgcgtgggttcgaaccccacttctggtagaccttttccccatcagtttttataaacttacaca

SEQ ID NO: 236 - DNA, 172 bp, synthetic construct
  source 1..172: mol_type = other DNA
gccaggggtgtggccatacaggtttatagtggttagtagagacaagtaagtgggtgacgaggtggccgagtggttaaggcgatggactctaaatccattgtgctctgcacacgtgggttcgaatcccatcctcgtcggaccttttccccatcagtttttataaacttacaca

SEQ ID NO: 237 - DNA, 172 bp, synthetic construct
  source 1..172: mol_type = other DNA
gccaggggtgtggccatacaggtttatagtggttagtagagacaagtaagtgggtgtcacggtggccgagtggttaaggcgttggacttcaaatccaatggggtttccccgcacaggttcgaatcctgttcgtgacggaccttttccccatcagtttttataaacttacaca

SEQ ID NO: 238 - DNA, 172 bp, synthetic construct
  source 1..172: mol_type = other DNA
gccaggggtgtggccatacaggtttatagtggttagtagagacaagtaagtgggtgtagtcgtggccgagtggttaaggcgatggactttaaatccattggggtttccccacgcaggttcgaatcctgccgactacggaccttttccccatcagtttttataaacttacaca

SEQ ID NO: 239 - DNA, 163 bp, synthetic construct
  source 1..163: mol_type = other DNA
gccaggggtgtggccatacaggtttatagtggttagtagagacaagtaagtgggtgcctgggtagctcagtcggtagagcatcagactttaaatctgagggtccagggttcaagtccctgtccaggcggaccttttccccatcagtttttataaacttacaca

SEQ ID NO: 240 - DNA, 138 bp, synthetic construct; identical to SEQ ID NO: 203
  source 1..138: mol_type = other DNA

SEQ ID NO: 241 - DNA, 138 bp, synthetic construct; identical to SEQ ID NO: 217
  source 1..138: mol_type = other DNA

SEQ ID NO: 242 - DNA, 138 bp, synthetic construct; identical to SEQ ID NO: 218
  source 1..138: mol_type = other DNA

SEQ ID NO: 243 - DNA, 139 bp, synthetic construct
  source 1..139: mol_type = other DNA
agcgctccggtttttctgtgctgaacctcaggggacgccgacacacgtacacgtcccttcgatagctcagctggtagagcggaggactttagatccttaggtcccgggttcgattcccggtcgaaggagtccttttttt

SEQ ID NO: 244 - DNA, 139 bp, synthetic construct
  source 1..139: mol_type = other DNA
agcgctccggtttttctgtgctgaacctcaggggacgccgacacacgtacacgtcccttcgatagctcagctggtagagcggaggactctagatccttaggtcccgggttcgattcccggtcgaaggagtccttttttt

SEQ ID NO: 245 - DNA, 148 bp, synthetic construct
  source 1..148: mol_type = other DNA
agcgctccggtttttctgtgctgaacctcaggggacgccgacacacgtacacgtcgacgaggtggccgagtggttaaggcgatggactctaaatccattgtgctctgcacacccgggttcgaatcccggcctcgtcggtccttttttt

SEQ ID NO: 246 - DNA, 148 bp, synthetic construct
  source 1..148: mol_type = other DNA
agcgctccggtttttctgtgctgaacctcaggggacgccgacacacgtacacgtcgtcacggtggccgagtggttaaggcgttggacttcaaatccaatggggtttccccgcccgggttcgaatcccggtcgtgacggtccttttttt

SEQ ID NO: 247 - DNA, 148 bp, synthetic construct
  source 1..148: mol_type = other DNA
agcgctccggtttttctgtgctgaacctcaggggacgccgacacacgtacacgtcgtagtcgtggccgagtggttaaggcgatggactttaaatccattggggtttccccacccgggttcgaatcccggcgactacggtccttttttt

SEQ ID NO: 248 - DNA, 139 bp, synthetic construct
  source 1..139: mol_type = other DNA
agcgctccggtttttctgtgctgaacctcaggggacgccgacacacgtacacgtcgggggtatagctcagtgggtagagcatttgacttcagatcaagaggtcccgggttcaaatcccggtgccccctgtccttttttt

SEQ ID NO: 249 - DNA, 172 bp, synthetic construct
  source 1..172: mol_type = other DNA
gccaggggtgtggccatacaggtttatagtggttagtagagacaagtaagtgggtgacgaggtggccgagtggttaaggcgatggactctaaatccattgtgctctgcacacccgggttcgaatcccggcctcgtcggaccttttccccatcagtttttataaacttacaca

SEQ ID NO: 250 - DNA, 172 bp, synthetic construct
  source 1..172: mol_type = other DNA
gccaggggtgtggccatacaggtttatagtggttagtagagacaagtaagtgggtgtcacggtggccgagtggttaaggcgttggacttcaaatccaatggggtttccccgcccgggttcgaatcccggtcgtgacggaccttttccccatcagtttttataaacttacaca

SEQ ID NO: 251 - DNA, 172 bp, synthetic construct
  source 1..172: mol_type = other DNA
gccaggggtgtggccatacaggtttatagtggttagtagagacaagtaagtgggtgtagtcgtggccgagtggttaaggcgatggactttaaatccattggggtttccccacccgggttcgaatcccggcgactacggaccttttccccatcagtttttataaacttacaca

SEQ ID NO: 252 - protein, 9 aa, synthetic construct
LEYNFNSHR

SEQ ID NO: 253 - protein, 16 aa, synthetic construct
  REGION 9: note = X represents any amino acid EXCEPT R.
LEYNFNSHXVYITADK

SEQ ID NO: 254 - DNA, 139 bp, synthetic construct; identical to SEQ ID NO: 40
  source 1..139: mol_type = other DNA

SEQ ID NO: 255 - DNA, 55 bp, synthetic construct; identical to SEQ ID NO: 91
  source 1..55: mol_type = other DNA

SEQ ID NO: 256 - DNA, 73 bp, synthetic construct
  source 1..73: mol_type = other DNA
ggctctgtggcgcaatggatagcgcattggacttcaaattcaaaggttgtgggttcgagtcccaccagagtcg

SEQ ID NO: 257-258 - skipped (2 entries)

SEQ ID NO: 259 - DNA, 17 bp, synthetic construct
  source 1..17: mol_type = other DNA
gtgggttcgagtcccac

SEQ ID NO: 260-261 - skipped (2 entries)

SEQ ID NO: 262 - DNA, 72 bp, synthetic construct
  source 1..72: mol_type = other DNA
ggggaccatagaaggagcgttctcgtcgcggtccctgtcaggctcgtcctgcgaatccttctaccacatgct

SEQ ID NO: 263 - DNA, 207 bp, synthetic construct
  source 1..207: mol_type = other DNA
agcgctccggtttttctgtgctgaacctcaggggacgccgacacacgtacacgtcggctctgtggcgcaatggatagcgcattggacttcaaattcaaaggttgtgggttcgagtcccaccagagtcgggggaccatagaaggagcgttctcgtcgcggtccctgtcaggctcgtcctgcgaatccttctaccacatgctttttttt

SEQ ID NO: 264 - RNA, 74 nt, synthetic construct
  source 1..74: mol_type = other RNA
ggctctggtggcgcaatggatagcgcattggacttcaaattcaaaggttgtgggttcgagtcccaccagagtcg

SEQ ID NO: 265 - RNA, 82 nt, synthetic construct
  source 1..82: mol_type = other RNA
accagaatggccgagtggttaaggcgttggacttcagatccaatggattcatatccgcgtgggttcgaacccacttctggta